(19) United States
(12) Patent Application Publication
(10) Pub. No.: US 2013/0158103 A1
(43) Pub. Date: Jun. 20, 2013

(54) Method of Tissue-Selective Targeted Gene Transfer

(71) Applicant: The Curators of the University of Missouri, Columbia, MO (US)

(72) Inventor: Rajiv R. Mohan, Columbia, MO (US)

(73) Assignee: THE CURATORS OF THE UNIVERSITY OF MISSOURI, Columbia, MO (US)

(21) Appl. No.: 13/683,944

(22) Filed: Nov. 21, 2012

Related U.S. Application Data
(60) Provisional application No. 61/629,679, filed on Nov. 23, 2011.
(60) Provisional application No. 61/629,680, filed on Nov. 23, 2011.

(51) Int. Cl. A61K 48/00; A61K 38/39

(52) U.S. Cl. CPC A61K 48/0075; A61K 38/39
USPC 514/44 R

(57) ABSTRACT

The present invention relates to methods of delivering a gene, such as a therapeutic gene, to a desired area of stroma of a cornea that involves removing the corneal epithelium and dehydrating the cornea. Certain aspects of the present invention relate to methods of treating corneal scarring by delivering a TGFβ-antagonizing gene packaged in a viral vector.

### CROSS REFERENCE TO RELATED APPLICATIONS

This application claims the benefit of U.S. Provisional Application No. 61/629,679, filed Nov. 23, 2011, and U.S. Provisional Application No. 61/629,680, filed Nov. 23, 2011, both of which are hereby incorporated herein by reference in their entireties.

### STATEMENT REGARDING FEDERALLY SPONSORED RESEARCH

This invention was made with Government support under Grant No. R01 EY017294 awarded by the National Institutes of Health (NIH). The Government has certain rights in the invention.

### INCORPORATION OF SEQUENCE LISTING

A computer readable form of the Sequence Listing is provided herein, contained in the file named “109069_Seq List_ST25.txt”, which is 8,945 bytes (measured in operating system MS-Windows), created on Nov. 21, 2012, and incorporated herein by reference in its entirety. This Sequence Listing consists of SEQ ID NO: 1-6.

### BACKGROUND OF THE INVENTION

Corneal scarring is a leading cause of blindness. A variety of factors such as infection, trauma, chemical and mechanical injury to the eye have been shown to cause fibrosis or scarring in the cornea (Qazi Y, et al. Brain Res Bull. 2010; 8:198-210; Hassell J R and Birk D E. Exp Eye Res. 2010; 91:326-35; Wilson S E, et al. Prog Retin Eye Res. 2001; 20:625-37). It is also a common complication of laser eye surgery that is used frequently worldwide to correct refractive errors and corneal dystrophies. Among popular eye surgeries, photorefractive keratectomy (PRK) has been implicated to induce corneal haze in a significant portion of high myopic patients due to epithelial injury and abnormal wound healing in the cornea following excimer laser utilization (Seiler T and McDonnell P J. Surv Ophthalmol. 1995; 40:89-118; Netto M V, et al. Cornea 2005; 24:509-22). Current conventional drug therapies for treating corneal scarring require repeated applications, provide short-term benefit, cause many side effects, and are often ineffective in eliminating corneal scarring. No efficacious long-term treatments for curing corneal scarring without causing side effects are available.

Various viral and non-viral vectors have been tested for delivering genes in the cornea (Mohan R R, et al. Prog Retin Eye Res. 2005; 24:537-59; Sharma A, et al. Ocular Surface Gene Therapy. In: Besharse J, Dana R, Dartt D A, editors. Encyclopedia of the eye. Elsevier; 2010. p. 185-94; Mohan R R, et al. Exp Eye Res. 2003; 76:373-83; Sharma A, et al. Brain Res Bull. 2010; 81:273-78; Sharma A, et al. Exp Eye Res. 2010; 91:440-48; Buss D G, et al. Vet Ophthalmol. 2010; 13:301-16). Among various gene transfer vectors tested for corneal gene therapy, viral vectors have emerged as favored vectors as they exhibited high transgene expression for longer durations (Mohan R R, et al. Prog Retin Eye Res 2005; 24:537-59; Sharma A, et al. Ocular Surface: Gene Therapy. In: Besharse J, Dana R, Dartt D A, editors. Encyclopedia of the eye. Elsevier; 2010. p. 185-194; Klausner E A, et al. J Control Release 2007; 124:107-33). Evidence for treating corneal scarring with gene therapy was demonstrated by delivering herpes simplex virus thymidine kinase gene after keratectomy with retroviral vector, followed by topical application of ganciclovir in PPK-induced corneal fibrosis rabbit model (Behrens A and McDonnell P J. Adv Exp Med. Biol. 2002; 506(Pt B):1315-21; Behrens A, et al. Invest Ophthalmol V is Sci. 2002; 43:968-77). Among viral gene therapy vectors, retrovirus and adenovirus have been shown to cause multiple side effects, raising safety concerns and sharply limiting their clinical application.

Adeno-associated virus (AAV) vectors are found to be highly efficient and safe for delivering foreign genes in rodent, rabbit, equine, and human cornea in vitro and in vivo (Mohan R R, et al. Prog Retin Eye Res 2005; 24:537-59; Sharma A, et al. Ocular Surface: Gene Therapy. In: Besharse J, Dana R, Dartt D A, editors. Encyclopedia of the eye. Elsevier; 2010. p. 185-194; Mohan R R, et al. Exp Eye Res 2003; 76:373-83; Sharma A, et al. Brain Res Bull 2010; 81:273-8; Sharma A, et al. Exp Eye Res 2010; 91:440-8; Buss D G, et al. Vet Ophthalmol. 2010; 13:301-6). A varied degree of tissue-selective tropism among AAV serotypes has also been observed for the cornea like other tissues (Sharma A, et al. Brain Res Bull 2010; 81:273-8; Sharma A, et al. Exp Eye Res 2010; 91:440-8; Buss D G, et al. Vet Ophthalmol. 2010; 13:301-6; Surace EM and Auricchio A. Vision Res 2008; 48:353-9).

The stroma constitutes 90% of the corneal tissue and its cellular components play an important role in maintaining corneal transparency, function, and pathology. The stroma is affected in a variety of corneal diseases such as graft rejection, haze, neovascularization, herpes keratitis, fibrosis, and scarring. Gene therapy treatments without any side effects to treat stromal corneal disorders require localized expression of therapeutic genes into keratocytes and/or the stroma. Numerous recombinant viruses and lipids have been tested administering variable volume, concentration, strength, duration, and frequency of vector in the cornea via microinjection, topical, electroporation, ultrasound, or gene gun (Hao J, et al. Brain Res Bull 2010; 81:256-61). However, untargeted and uncontrolled gene delivery remains a major challenge as does the efficiency of gene transfer.

The molecular mechanism of corneal fibrosis has been extensively studied but is still not fully defined. Scores of studies suggest the role of numerous growth factors and cytokines in the pathophysiology of corneal scarring (Tandon A, et al. Curr Mol. Med. 2010; 10:565-78; Jester J V, et al. Prog. Retin. Eye Res. 1999; 18:311-56). Out of many cytokines, transforming growth factor (TGFβ), released from corneal epithelium following eye injury, has been demonstrated to play a central role in the genesis of corneal fibrosis by promoting myofibroblast formation, as well as synthesis of extracellular matrix (ECM) and cytoskeletal proteins (Mohan R R, et al. PloS one 2011; 6:e18771; Tandon A, et al. Curr Mol. Med. 2010; 10:565-78; Jester J V, et al. Prog. Retin. Eye Res. 1999; 18:311-56). Support for this notion was provided by studies performed by Jester et al. that showed significant inhibition of corneal fibrosis in rabbit eyes by topical application of TGFβ neutralizing antibodies (Bainbridge J W, et al. N Engl J. Med. 2008; 358:2231-9).

There remains a need to provide new and improved therapeutic reagents and vector-delivery methods for targeted and controlled gene delivery to the corneal stroma in vivo and to treat corneal scarring and fibrosis.

### SUMMARY OF THE INVENTION

The present invention is drawn to new and improved methods for delivering a gene to a desired area of stroma of a cornea. Such a method comprises first preparing the cornea for targeted gene delivery by: (i) removing corneal tissue to expose at least a portion of the corneal stroma, and (ii) dehydrating the exposed portion of the stroma. After the cornea is prepared, a viral vector that comprises the gene is applied to at least the dehydrated portion of the stroma. In certain embodiments, excess viral vector is removed after its application. In certain embodiments, the viral vector is an AAV vector in solution. In certain embodiments, the corneal tissue is removed by mechanical scraping. In certain embodiments, following preparation of the cornea, a physical barrier is placed on the cornea that encompasses the desired surface area of stroma of the cornea to which the gene is to be delivered. The viral vector is applied in a solution to the area encompassed by the physical barrier.

The present invention is also drawn to new and improved methods of treating corneal scarring. Such a method comprises applying a viral vector that comprises a TGFβ-antagonizing gene to the stroma of a cornea. In certain embodiments, the viral vector is an AAV vector, such as an AAV5. In certain embodiments, the TGFβ-antagonizing gene is decorin. In certain embodiments, the viral vector and TGFβ-antagonizing gene is delivered to a desired area of corneal stroma according to the targeted gene delivery method described herein.

These and other aspects of the invention will become apparent to those skilled in the art in light of the following disclosure and accompanying drawings.

### BRIEF DESCRIPTION OF THE DRAWINGS

FIG. 1 shows the effect of corneal drying on vector absorption in mice, rabbit, and human corneas. Corneas were subjected to zero (0 sec), one (10 sec), two (20 sec), three (30 sec), or five (50 sec) rounds of warm air drying and 2 μl (mouse) or 50 μl (rabbit and human) of vector/BSS was applied onto corneas for 2 minutes immediately after drying. The amount of vector absorbed by the corneas was calculated by subtracting unabsorbed BSS/vector volume from the total applied BBS/vector volume. The results are expressed in percent as mean±SEM. *p<0.001 as compared to 0 sec or 10 sec; ψ P p<0.001 as compared to 20 sec.

FIGS. 2A, 2B, 2C, and 2D are representative images showing alkaline phosphatase marker gene delivery detected on day 14 in naïve (A) and AAV-treated mouse corneas in vivo that received warm air for 20 seconds (B), 30 seconds (C), or 50 seconds (D). Scale bar denotes 50 μm.

FIG. 3 shows the digital quantification of delivered marker gene expression detected at day 14 in mouse corneas in vivo. These corneas received 2 μl of AAV8 vector immediately after 0, 20, 30, or 50 seconds of drying. The corneas that underwent 50 seconds of drying showed the highest level of gene expression followed by 30 seconds and then the 20 seconds group. *p<0.05 as compared to 20 seconds; ψ p<0.05 as compared to 30 seconds.

FIGS. 4A, 4B, 4C, and 4D are representative images of H&E staining showing histology of mouse corneas subjected to 0 second (A), 20 seconds (B), 30 seconds (C), or 50 seconds (D), of air drying and collected on day 14 after vector application. Corneas exposed to 30 seconds of drying showed normal morphology whereas corneas subjected to 50 seconds of drying showed moderate morphological changes in anterior stroma. Scale bar denotes 50 μm.

FIGS. 5A, 5B, 5C, and 5D are representative immunohistochemistry images showing CD1 1b+ cells in naïve (A) and AAV-treated mouse corneas subjected to warm air for 20 seconds (B), 30 seconds (C), or 50 seconds (D), and collected on day 14 after vector application. Scale bar denotes 50 μm.

FIGS. 6A, 6B, and 6C are representative images showing vector application in mouse cornea in vivo (A), rabbit cornea in vivo (B), and human cornea ex vivo (C), showing a representative example of an embodiment of the cloning cylinders technique of the invention.

FIGS. 7A, 7C, and 7E are representative stereomicroscopy images and FIGS. 7B and 7D are representative biomicroscopy images showing haze levels in no decorin-delivered control (A, B) and decorin-delivered (C, D) rabbit corneas. Haze was produced with PRK surgery, AAV5 viral titer (6.5×10¹² vg/ml) was applied immediately onto the cornea for 2 minutes after PRK, and corneal tissues were imaged 4 weeks after PRK surgery (at this time peak in haze is reported). Graph (F) depicts the quantification of corneal haze. Significantly less haze (p<0.01) was observed in decorin delivered corneas compared to no decorin-delivered corneas. No haze was observed in contralateral naïve rabbit corneas (E). dcn=decorin.

FIG. 8 shows the effect of AAV5-mediated decorin gene delivery on markers of corneal fibrosis as detected by immunostaining in rabbit corneal tissue. Immunohistochemistry showing levels of αSMA in no-decorin-delivered control (A) and decorin-delivered (B) rabbit corneal tissue, f-actin in no-decorin-delivered control (C) and decorin-delivered (D) rabbit corneal tissue and fibronectin in no-decorin-delivered control (E) and decorin-delivered (F) rabbit corneal tissues collected at 4 weeks after PRK-induced corneal fibrosis. Decorin treated corneas showed significant decreases in αSMA (p<0.001) and notable decreases in f-actin and fibronectin levels in the stroma compared to control corneas. Scale bar denotes 100 μm.

FIG. 9 shows the effect of AAV5-mediated decorin gene therapy on fibrosis in rabbit corneal tissues quantified by counting αSMA-positive cells/400× magnification. Decorin treatment significantly (p<0.001) decreased αSMA positive cells.

FIG. 10 shows Western blot analysis for αSMA to detect the effect of AAV5-mediated decorin gene therapy on fibrosis in rabbit corneas. A significant (75-86%) decrease in expression of αSMA was detected in decorin-delivered corneas compared to control corneas suggesting that AAV5-mediated decorin gene therapy is highly efficient in treating corneal fibrosis. 6-actin was used to confirm equal loading of protein in each well and normalization of the data.

FIG. 11 shows the effect of AAV5-decorin gene therapy on immune response in rabbit corneas using CD11b and F4/80 immunocytochemistry. No significant difference in CD11b and F4/80 stained cells in the stroma of no-decorin-delivered and decorin-delivered rabbit corneas suggest that AAV5-mediated decorin gene therapy appears not to induce immune response in the cornea. Alkali-burn rabbit corneal tissue sections of 4 hours were used as positive controls. Scale bar denotes 100 μm.

FIG. 12 shows the effect of AAV5-decorin gene delivery on rabbit corneal toxicity tested using TUNEL assay. Detection of low (2-5) TUNEL+ cells in the stroma of no-decorin-delivered control (A) and decorin-delivered (B) rabbit corneal sections, and comparable number of TUNEL+ cells in the epithelium, and no change in overall keratocyte density in the stroma determined with DAPI+ nuclei suggest that decorin gene therapy is non-cytoxic and safe to the cornea. Scale bar denotes 100 μm.

FIG. 13 shows the effect of decorin gene therapy on collagen fibril diameter determined by transmission electron microscopy imaging of no-decorin-delivered control (A) and decorin-delivered (B) rabbit corneas. The graph (C) shows quantification of collagen fibril diameter in nanometers. No significant difference in the collagen fibril diameter was detected between control and decorin-delivered rabbit corneas. This suggests that AAV5-delivered decorin did not alter collagen fibril status in the rabbit cornea.

FIG. 14 shows SLRPs categorized into five distinct Classes represented by Roman numerals I-V. Various SLRPs are found on the same chromosome indicated by the numbers within parentheses.

FIG. 15 is a schematic representation showing structural domains of the decorin protein. The four structural domains are marked with roman numerals. Domain I is comprised of the signal and propeptide which are absent in the mature decorin protein. Domains II, III, and IV make up the mature decorin protein. Domain II consists of the amino terminus including the glycosaminoglycan (GAG) chain with a binding site for thrombospondin (Thbs) and other GAGs. Domain III is comprised of the 40 kDa core protein and contains leucine-rich repeats (LRR; white and black boxes) with the amino acid sequence LXXLXLXXNXL (SEQ ID NO: 1). Domain III also has binding sites for collagen (col; high affinity), thrombospondin, TGFβ, and the heparinbinding domain of fibronectin (Fn). In addition it also contains N-linked oligosaccharides (oligos). Domain IV is the carboxyl terminus domain and harbors binding sites for fibronectin (cell-binding domain) and collagen (low affinity).

FIG. 16 shows representative in vivo fluorescence stereomicrograph (A) and tissue sections (B-D) of rabbit corneas showing AAV5-mediated GFP gene expression at 3-day and 2-week time points. Topical application of AAV5-GFP vector selectively transduced anterior keratocytes (arrows) located beneath the epithelium (C, D). No transgene expression was detected in control corneas (A). The rabbit corneas collected at 4-week and 16-week showed similar levels of GFP expression with immunostatining. Nuclei are stained with DAPI. Scale bar denotes 100 μm.

FIG. 17 shows representative western blot (upper panel) and digital quantification (lower panel) of delivered transgene in rabbit corneas at various time points. The delivered transgene expression first detected at 3-day, peaked at 7-day and maintained up to longest tested 16-week time point. β-actin was used to confirm equal loading of protein in each well and normalization of data. * p<0.05 compared to BSS-treated controls and 3-day time point, and τ p<0.01 compared to BSS-treated control.

FIG. 18 shows representative three-dimensional confocal microscopy images showing spatial localization of GFP-expressing keratocytes (arrows) in whole-mount rabbit corneas exposed to AAV5. Corneas collected 3 days (A) and 2 weeks (B) after topical application of AAV5-GFP vector showed GFP-positive keratocytes beneath the epithelium in the anterior stroma. Nuclei are stained with DAPI. Scale bar denotes 75 μm.

FIG. 19 shows a slot blot showing GFP gene copy number in AAV5-GFP treated rabbit corneas. Densitometric comparison detected 108-1010 copies of GFP gene in AAV5 GFP-treated rabbit corneas. Left lane shows standard plot of GFP plasmid DNA copies blotted at 10-fold dilution series. The right lane shows delivered GFP DNA copies detected in rabbit corneas collected 2-week after AAV5-GFP application.

FIG. 20 shows representative images of corneal sections showing efficacy of AAV5-mediated transgene delivery in fibrotic rabbit corneas. Corneal fibrosis was produced by PRK laser surgery which induces transdifferentiation of keratocytes (myofibroblasts staining for αSMA). Keratocytes expressing delivered GFP are shown (arrow), transdifferentiation keratocytes (myofibroblasts) expressing GFP and a-smooth muscle actin are shown (arrowheads), and transdifferentiation keratocytes (myofibroblasts) expressing a-smooth muscle actin are shown (Cut arrowheads). Nuclei are stained with DAPI. Scale bar denotes 100 μm.

FIG. 21 shows representative in vivo images of corneal tissue sections showing efficacy of AAV5-mediated transgene delivery in neovascularized rabbit cornea. Keratocytes expressing delivered GFP are shown (arrowheads). Blood vessels are stained with lectin (arrows). Nuclei are stained with DAPI. Scale bar denotes 100 μm.

FIG. 22 shows representative slit lamp microscopy and images demonstrating safety of tested AAV5 to the cornea. No inflammation, redness, water discharge, swelling, etc was observed in BSS-treated control (A) or AAV5-treated corneas (B). Hematoxylin and eosin staining of corneal tissue sections (D) obtained from AAV5-treated rabbit eyes showed corneal morphology comparable to control corneas (C). Panels A-D shows data of 1-day time point. Similar observations were recorded for other tested time points. Scale bar denotes 100 μm.

FIG. 23 shows PCR analysis showing delivery of plasmid containing decorin gene into several identified HSF clones. After reverse transcribing total RNA from HSF cultures transfected with pcDNA3.1-decorin (6 clones) or naked vector (2 clones) were amplified using primers sets prepared from decorin and vector DNA sequences. An expected 1.2 Kb band for decorin was detected after 21 cycles of amplification. The “L” represents 100 bp DNA ladder and “W” is negative control reaction ran without cDNA.

FIG. 24 shows immunoblotting analysis showing expression of decorin in two decorin-transfected HSF clones. Total protein extracts prepared from HSF transfected with pcDNA3.1-decorin or naked vector were analyzed. A protein band at 43 kDa, specific for decorin, was detected in decorin-transfected HSF clones. The naked vector transfected HSF clone did not show any band. Commercially available decorin-transfected and non-transfected 293T cell lysates were used as positive and negative controls, respectively.

FIG. 25 shows representative phase-contrast light microscopy images showing un-transfected and decorin-transfected HSF clones phenotype. The 80-90% confluent decorin-transfected (left panel) HSF clone showed morphology similar to un-transfected HSF clones (right panel). This suggests that decorin gene transfer does not alter HSF phenotype. Scale bar denotes 50 μm.

FIG. 26 shows Real-time quantitative PCR showing analysis of pro-fibrogenic genes, fibronectin, alpha collagen type-I, -III, and -IV expression in un-transfected, naked vector-transfected, and decorin-transfected HSF cultures grown in presence or absence of TGFβ1 (1 ng/ml) under serum-free conditions. The TGFβ1 caused 1.1-4.8-fold increase in tested genes in untransfected or naked-vector transfected HSF cultures grown in presence of TGFβ1 compared to cultures grown in absence of TGFβ1. Decorin transfection showed statistically significant inhibition of TGFβ1-induced expression of fibronectin, collagen type-I, -III and -IV. *=p<0.01 and τ=p<0.05 (−TGFβ1 naked-vector transfected HSF vs +TGFβ1 naked-vector transfected HSF), **=p<0.001 and π=p<0.01 (−TGFβ1 naked vector-transfected vs +TGFβ1 decorin-transfected HSF). No significant differences in the RNA levels of fibronectin, collagen type I, III or IV were detected between the un-transfected normal and naked-vector transfected HSF.

FIG. 27 shows Real-time quantitative PCR showing analysis of SMA RNA levels in un-transfected and decorin-transfected HSF cultures grown in presence or absence of TGFβ1 (1 ng/ml) under serum-free conditions. A statistically significant more than 8-fold (p<0.01) increase in the SMA gene was detected in un-transfected HSF cultures grown in the presence of TGFβ1 compared to cultures grown in the absence of TGFβ1. Decorin transfected HSF samples showed a statistically significant decrease (>6-fold; p<0.001) in SMA compared to un-transfected HSF cultures grown under similar conditions. The * indicates a p<0.01 compared to negative control (no TGFβ1) and the ** indicates a p<0.001 of un-transfected HSF versus decorin-transfected HSF grown in the presence of TGFβ1.

FIG. 28 shows Representative images showing immunocytochemistry of SMA, a myofibroblast marker, done in un-transfected HSF and decorin-transfected HSF cultures grown in presence or absence of TGFβ1 (1 ng/ml) under serum-free conditions. Decorin-transfected HSF (D) showed a significant decrease (79%, p<0.01) in SMA-positive cells compared to the un-transfected HSF (B) grown in the presence of TGFβ1. No SMA-positive cells were detected in un-transfected (A) or decorin-transfected cultures (C) grown in the absence of TGFβ1. Scale bar denotes 50 μm.

FIG. 29 shows Western blot analysis (panel A) and digital quantification (panel B) of SMA expression detected in un-transfected HSF and decorin-transfected HSF grown in the presence or absence of TGFβ1 under serum-free conditions. Decorin-transfected HSF cultures showed a significant decrease in SMA-expression compared to un-transfected HSF grown in the presence of TGFβ1 (−83%, p<0.01). Neither un-transfected nor decorin-transfected HSF cultured in the absence of TGFβ1 expressed SMA.

FIG. 30 shows representative images showing gene transfer into keratocytes of the mouse cornea in vivo with AAV9. The alkaline phosphatase cytochemical staining shows gene transfer in the stroma of mouse cornea collected 4, 14 and 30 days (d) after AAV9 administration. Control (con) represents corneal sections treated with naked viral vector. Nuclei are stained with nuclear red fast solution. Scale bar denotes 50 μm.

FIG. 31 shows representative images showing gene transfer into keratocytes of the mouse cornea in vivo with AAV8. The alkaline phosphatase cytochemical staining shows gene transfer in the stroma of mouse cornea collected 4, 14 and 30 days (d) after AAV8 administration. Control (con) represents corneal sections treated with naked viral vector. Nuclei are stained with nuclear red fast solution. Scale bar denotes 50 μm.

FIG. 32 shows representative images showing gene transfer into keratocytes of the mouse cornea in vivo with AAV6. The alkaline phosphatase cytochemical staining shows gene transfer in the stroma of mouse cornea collected 4, 14 and 30 days (d) after AAV6 administration. Control (con) represents corneal sections treated with naked viral vector. Nuclei are stained with nuclear red fast solution. Scale bar denotes 50 μm.

FIG. 33 shows a digital measurement of transgene in the stroma of the mouse cornea delivered with AAV6, AAV8 or AAV9 vector. The AP-stained tissue area was quantified digitally by measuring pixels of AP-stained tissue in 4×104 μm² tissue area. ψ=p<0.05 (significance value of AAV8-mediated gene transfer noted at days 14 and 30 compared to day 4), *=p<0.01 (significance value of AAV9-mediated gene transfer noted at days 14 and 30 compared to day 4), φ=p<0.05 (significance value of AAV8-mediated gene transfer compared to AAV6 at day 30), Ω=p<0.01 (significance value of AAV9-mediated gene transfer compared to AAV6 at day 14 and 30).

FIG. 34 shows a quantification of functional activity of transgene delivered into mouse cornea in vivo with AAV6, AAV8 or AAV9 vector. The AP enzyme activity was quantified with spectrophotometer in corneal homogenates prepared 4, 14 and 30 days after vector application. ψ=p<0.05 (significance value of AAV8 or AAV9-mediated gene transfer noted at day 14 and 30 compared to AAV8, AAV9 or control groups of day 4), *=p<0.05 (significance value of AAV9-mediated gene transfer compared to AAV6 at day 14 and 30 and AAV8 compared to AAV6 at day 30).

FIG. 35 shows representative images showing AAV-mediated gene transfer in the human cornea ex vivo. The alkaline phosphatase cytochemical staining in panels show transgene delivery in the human cornea analyzed 5 days after AAV6, AAV8 or AAV9 treatment. Control (con) represents corneal sections treated with naked viral vector. Nuclei are stained with nuclear fast dye. Scale bar denotes 100 μm.

FIG. 36 shows representative images showing effect of titer on AAV9-mediated gene transfer in the mouse cornea in vivo. The alkaline phosphatase cytochemical staining shows gene transfer in the stroma of mouse cornea on day 14 after AAV9 application. Upper panel represents corneas treated with the naked AAV9 vector, the middle panel represents the corneas treated with high titer AAV9 vector (10⁹ genomic copies/μl) and lower panel represents corneas treated with low titer AAV9 vector (10⁶ genomic copies/μl). Nuclei are stained red with nuclear red fast solution. Scale bar denotes 100 μm.

FIG. 37 shows representative images of TUNEL assay showing cell death in mouse corneal sections of 4 days that received AAV6, AAV8 or AAV9 vector. Most of the TUNEL-positive cells were detected in the upper layers of the corneal epithelium that replenishes via apoptosis. Very few TUNEL-positive cells were observed in the mouse stroma where transgene was delivered by the AAV. Nuclei are stained with DAPI. Scale bar denotes 100 μm.

FIG. 38 shows representative images of immunostaining for CD11b and F4/80 of mouse cornea collected 4 days after AAV9 application. The AAV9-treated as well as control corneas showed few (up to 7) CD11b- or F4/80-positive cells in the stroma at 400× magnification. Similar CD11b and F4/80 staining pattern was noted in the cornea treated with AAV6 or AAV8 vector. The mouse corneas collected 12 h after epithelial scrape injury were used as a positive control and showed large number of CD11b+ or F4/80 cells in corneal sections. These sections do not show DAPI-stained epithelium due to scrape. Nuclei are stained with DAPI. Scale bar denotes 100 μm.

### DETAILED DESCRIPTION

In the following detailed description, numerous specific details are set forth in order to provide a thorough understanding of the invention. The following disclosed embodiments, however, are merely representative of the invention which may be embodied in various forms. It will be understood by those skilled in the art that the present invention may be practiced without these specific details. Thus, specific structural, functional, and procedural details described are not to be interpreted as limiting. In other instances, well-known methods, procedures, and components have not been described in detail so as not to obscure the present invention.

Headings are provided herein solely for ease of reading and should not be interpreted as limiting.

Unless otherwise defined, all technical and scientific terms used herein have the same meaning as commonly understood by one of ordinary skill in the art to which this invention pertains.

### Overview

One aspect of the present invention is drawn to a minimally invasive controlled-dehydration based topical vector-delivery technique that significantly increase vector-mediated transgene delivery and absorption in the corneal stroma in vivo, without compromising typical corneal morphology or function. In certain embodiments of the invention, a method is provided that combines a viral vector approach, topical application technique, and corneal drying technique to achieve the targeted and controlled delivery of therapeutic genes in the corneal stroma.

Without being bound by theory, it is believed that dehydration of the cornea increases vector absorption in the stroma tissue and in turn, enhances transgene expression in the cornea in vivo. Corneal injury due to forced-air drying is limited to minor compromise of corneal morphology. Thus, corneal injury may be avoided or minimized by adjustment of the temperature and flow rate and control of the duration of treatment for various species.

Certain embodiments of the invention are drawn to methods of delivering a gene to a desired area of stroma of a cornea, i.e., targeted gene delivery. Such methods involve preparing the cornea for gene delivery by first removing corneal tissue. In certain embodiments, the corneal tissue that is removed comprises the corneal epithelium. In certain embodiments, the corneal tissue that is removed may also comprise the Bowman's layer if present in the subject species. In certain embodiments, corneal tissue is removed to expose at least a portion of the corneal stroma. As used herein, the corneal stroma is “exposed” when at least the overlying corneal epithelium has been removed. The cornea, or at least a portion of the cornea comprising the corneal stroma, is then dehydrated as describe in detail herein. In certain embodiments, the portion of the corneal stroma that is dehydrated is at least the exposed portion of the stroma. Once the cornea is prepared, a viral vector is applied that has been assembled and packaged with the gene. In certain embodiments, the gene is a therapeutic gene. The viral vector is applied to the desired area of stroma of the cornea to which the gene delivery is to be targeted. In certain embodiments, the viral vector is applied to an exposed portion of the stroma, a dehydrated portion of the stroma, or an exposed and dehydrated portion of the stroma.

The gene that is delivered by the viral vector may be assembled and packaged with the viral vector using standard viral vector assembly and packaging protocols. After application of the viral vector, excess viral vector may be removed. The excess viral vector may be removed after a certain amount of time after application, such as after about 30 seconds, after about 45 seconds, after about 60 seconds, after about 90 seconds, after about 120 seconds, after about 150 seconds, or after about 180 seconds. Longer durations may also be utilized or the excess viral vector may not be removed at all.

In certain embodiments, the viral vector to be applied is in solution. In certain embodiments, the viral vector is applied in a volume of from about 1 μl to about 100 μl of solution. In certain embodiments, the viral vector is applied in a volume of from about 2 μl to about 90 μl of solution. In certain embodiments, the viral vector is applied in a volume of from about 2 μl to about 80 μl of solution. In certain embodiments, the viral vector is applied in a volume of from about 2 μl to about 70 μl of solution. In certain embodiments, the viral vector is applied in a volume of from about 2 μl to about 60 μl of solution. In certain embodiments, the viral vector is applied in a volume of from about 2 μl to about 50 μl of solution. In certain embodiments, the viral vector is applied in a volume of from about 2 μl to about 40 μl of solution. In certain embodiments, the viral vector is applied in a volume of from about 2 μl to about 30 μl of solution. In certain embodiments, the viral vector is applied in a volume of from about 2 μl to about 20 μl of solution. In certain embodiments, the viral vector is applied in a volume of from about 100 μl to about 1,000 μl of solution. One of skill in the art will understand that the volume used will in part depend upon factors such as the concentration (e.g., titer such as expressed as vg/ml) of the viral vector in the solution and the species/size of the cornea and size of the desired area to target.

Viral Vectors

Any suitable viral vectors may be selected for gene delivery. Representative examples of viral vectors include Adenovirus, adeno-associated virus (AAV), herpesvirus, lentivirus, and retrovirus In certain embodiments, the preferred viral vectors are adeno-associated virus (AAV) vectors due to safety and efficacy considerations. Representative examples of AAV vectors include AAV5, AAV6, AAV8, and AAV9. In certain embodiments, the AAV vector is AAV5. In certain embodiments, the viral vector titer is from about 1×10⁸ vg/ml to about 1×10¹³ vg/ml. In certain embodiments, the viral vector titer is from about 1×10⁸ vg/ml to about 6.5×10¹² vg/ml. In certain embodiments, the viral vector titer is from about 1×10⁸ vg/ml to about 1×10⁹ vg/ml. In certain embodiments, the viral vector titer is from about 1×10⁸ vg/ml to about 1×11⁸ vg/ml.

Removal of the Corneal Tissue

When preparing the cornea, the removal of the corneal tissue is preferred for achieving efficient and targeted gene delivery into the stroma, since overlying corneal tissue, such as the corneal epithelium, acts as a strong barrier to topical gene delivery. Corneal tissue, such as the corneal epithelium, may be removed via the mechanical scraping method as this technique is routinely used clinically in refractive laser surgical procedures such as photorefractive keratectomy, laser epithelial keratomileusis, etc. Mechanical removal of epithelium is known to induce keratocyte apoptosis, inflammation, and wound healing in the cornea (Mohan R R, et al. Exp Eye Res 2003; 76:71-87; Wilson S E, et al. Adv Exp Med Biol 2002; 506(Pt B):821-6). Release of cytokines and growth factors following epithelial injury ignite a transient wound healing response in the cornea (Mohan R R, et al. Exp Eye Res 2003; 76:71-87; Wilson S E, et al. Adv Exp Med Biol 2002; 506(Pt B):821-6; Jester J V, et al. Prog Retin Eye Res 1999; 18:311-56).

Corneal Drying

The hydration and porosity of the cornea regulate its transparency (Maurice D M and Riley M V. The cornea. In: Graymore C, editor. Biochemistry of the Eye. New York: Academic Press; 1970. p. 1-103). The cornea constantly absorbs fluid from the aqueous humor and limbal blood vessels and becomes hazy if not pumped out by the endothelium (Maurice DM. Cornea and sclera. In: Dayson D, editor. The eye. Vol 1b. 3rd ed. London: Academic Press; 1984. p. 1-158). The corneas of human cadavers are often hazy due to absorption of aqueous humor but their transparency can be restored by incubating tissue in warm and ventilated chamber at 31° C. (Pets E, et al. Int Ophthalmol 2008; 28:155-63). Furthermore, the drying of the cornea with a hair dryer is a known conventional treatment for Fuchs' dystrophy among patients in eye clinic (Suh L H and Emerson M V. Fuchs endothelial dystrophy: pathogenesis and management. In: Reinhard T, Larkin F, editors. Cornea and external disease. Jun A S. Berlin: Springer-Verlag; 2008. p. 1-13).

One aspect of the present invention is drawn to the dehydration of the cornea. Without being bound by theory, it is believed that drying of the cornea for short duration augments fluid absorbing capacity of the cornea until corneal hydration returns to the normal levels. Various dehydration methods may be used such as forced-air drying, sponges, and blotting paper. The forced-air corneal drying technique is an acceptable conventional treatment in clinical practice for corneal abnormalities such as Fuch's dystrophy (Suh L H and Emerson M V. Fuchs endothelial dystrophy: pathogenesis and management. In: Reinhard T, Larkin F, editors. Cornea and external disease. Jun A S. Berlin: Springer-Verlag; 2008. p. 1-13). In certain embodiments, the air or gas used to dehydrate the cornea is warm. Warm air or gas is that heated above ambient temperature, up to about 45° C. In certain embodiments, the air or gas is heated to from about 40° C. to about 45° C. In certain embodiments, the temperature of the drying air or gas is about 41° C. In certain embodiments, the air-flow rate of the air or gas used to dehydrate the cornea is about 6 meters/second to about 10 meters/second as measured by a digital Velocicheck anemometer (Model 8830; TSI Inc., Shoreview, Minn.), or a comparable rate. In certain embodiments, the air-flow rate of the air or gas used to dehydrate the cornea is about 6 meters/second to about 8 meters/second as measured by a digital Velocicheck anemometer (Model 8830; TSI Inc., Shoreview, Minn.) or a comparable rate. In certain embodiments, the air-flow rate of the air or gas used to dehydrate the cornea is about 6.8 meters/second as measured by a digital Velocicheck anemometer (Model 8830; TSI Inc., Shoreview, Minn.) or a comparable rate. In certain embodiments, a source of forced-air is held at a distance of about 7 to 12 inches from the eye. In certain embodiments, a source of forced-air is held at a distance of about 8 inches from the eye. In certain embodiments, a source of forced-air is held and at an angle of from about 30° to 75° to the eye. In certain embodiments, a source of forced-air is held and at an angle of about 45° to the eye. In certain embodiments, a source of forced-air is held at a distance of about 8 inches for the eye and at an angle of about 45° to the eye. In certain embodiments, the source of forced-air is a hairdryer. The duration of drying may depend on the type and temperature of air or gas used, the air-flow rate, the distance and angle of the drying source, etc. In certain embodiments, the duration of drying is about 5 second, about 10 seconds, about 15 seconds, about 20 seconds, about 25 seconds, about 30 seconds, about 35 seconds, about 40 seconds, about 45 seconds, about 50 seconds, about 55 seconds, or about 60 seconds. In certain embodiments, the duration of the drying may be continuous. In certain embodiments, the duration of the drying may be interrupted, such as by intervals of about 5 seconds, about 10 seconds, about 15 seconds, about 20 seconds, about 25 seconds, or about 30 seconds, between rounds of drying. In certain embodiments, each round of drying is about 5 seconds, about 10 seconds, about 15 seconds, or about 20 seconds. One of skill in the art will recognize that the optimal temperature range, air-flow rate, duration of corneal drying, etc., may vary depending on the species.

Following dehydration and application of the targeted viral vector, the cornea will re-hydrate. Corneal hydration is critical for corneal function and clear vision (Manchester PT Jr. Trans Am Ophthalmol Soc 1970; 68:425-61; Monti D, et al; Int J Pharm 2002; 232:139-47) and is regulated by several processes and tissues including the epithelial barrier, the water holding capacity of the proteoglycans, endothelial water-pumping mechanisms, intraocular pressure and evaporation of fluid from the corneal surface. In addition to these factors many agents such as benzalkonium chloride, cetylpyridinium chloride, EDTA, polyethoxylated castor oil, sodium deoxycholate, etc., present in ophthalmic topical formulations have been shown to influence corneal hydration (Manchester PT Jr. Trans Am Ophthalmol Soc 1970; 68:425-61; Kidron H, et al. Pharm Res 2010; 27:1398-407). In certain embodiments, re-hydration of the cornea may be aided by one or more of these agents.

Physical Barrier

One aspect of the present invention is drawn to employing a physical barrier that is placed on the cornea to contain fluid over the area to be targeted for gene delivery. The physical barrier encompasses the desired surface area of stroma of the cornea to which the viral vector is to be targeted. One illustrative example of such a physical barrier includes a cloning cylinder. This significantly enhances transgene delivery into the targeted corneal stroma and prevents transgene delivery into untargeted tissues such as limbus, conjunctiva, sclera, etc. A typical physical barrier, when placed on the cornea, encompasses a generally two-dimensional area of the cornea that is to be targeted, and is preferably able to contain at least the entire volume of the viral vector solution that is to be applied. That said, the volume that the physical barrier is able to generally contain, when placed on the cornea, may be less than the entire volume of the viral vector solution that is to be applied. Representative amounts of viral vector solution useful in the invention are described elsewhere herein. Although the physical barrier tends to contain or prevent the spread of the viral vector solution, some leakage from out of the encompassed area may occur. In certain embodiments, the cornea is dehydrated before placement of the physical barrier.

In certain embodiments, the physical barrier is a cylinder with a diameter of from about 1 mm to about 10 mm. In certain embodiments, the physical barrier is a cylinder with a diameter of from about 3 mm to about 7 mm. In certain embodiments, the physical barrier is a cylinder with a diameter of about 3 mm, about 5 mm, or about 7 mm. In certain embodiments, the physical barrier is not a cylinder, but encompasses a two-dimensional area to contain fluid that is comparable the area enclosed by a cylinder described herein. In certain embodiments, the physical barrier is not a cylinder, but contains a volume to contain fluid that is comparable the volume of viral vector solution to be applied.

In certain embodiments, the viral vector may be targeted to a specific area of stroma of the cornea by placing the fluid containing the viral vector on a sponge that is placed on the area of stroma of the cornea to be targeted. In certain embodiments, the sponge is placed within a physical barrier.

### Treatment of Corneal Scarring

One aspect of the invention is drawn to a novel gene therapy method for treatment of corneal scarring that employs delivering a TGFβ-antagonizing gene to the cornea. In certain embodiments, the method comprises applying a viral vector that comprises a TFGβ-antagonizing gene to the stroma of a cornea. Any suitable viral vectors may be selected for assembly and packaging the TGFβ-antagonizing gene. Further it is understood that any gene chosen for assembling and packaging in a viral vector is expressible and that such gene is assembled and packaged such that the resulting viral vector comprising the gene is capable of expressing the gene when delivered to the cornea. Representative examples of viral vectors include Adenovirus, adeno-associated virus (AAV), herpesvirus, lentivirus, and retrovirus, all of which are known to expressibly deliver genes. In certain embodiments, the preferred viral vectors are adeno-associated virus (AAV) vectors due to safety and efficacy considerations. Representative examples of AAV vectors include AAV5, AAV6, AAV8, and AAV9. In certain embodiments, the method includes delivering AAV5 vectors containing TGFβ-antagonizing genes to the cornea. TGFβ-antagonizing genes that block TFGβ activity include, but are not limited to decorin, TGFβR2, and SMAD7.

Certain embodiments of the invention are drawn therapeutic formulations for treating corneal scarring comprising a viral vector assembled and packaged with a TGFβ-antagonizing gene that blocks TFGβ activity. Such assembled and packaged viral vectors of the therapeutic formulation are capable of expressing the TGFβ-antagonizing genes when delivered to the cornea. Representative examples of such genes include, but are not limited to decorin, TGFβR2, and SMAD7. Useful viral vectors include the AAV vectors disclosed herein.

In certain embodiments, tissue-selective gene delivery is used to deliver a TGFβ-antagonizing gene packaged in an AAV vector to the corneal stroma. The tissue-selective targeted delivery method comprises first preparing the cornea as described herein. AAV viral vectors comprising a TGFβ-antagonizing gene are then applied to the desired area of stroma of the cornea. The method may further comprise removing excess AAV vector. Localized delivery of the gene may employ, for example, a physical barrier such as a cloning cylinder to contain fluid placed on the cornea.

Decorin

Decorin is a small leucine-rich proteoglycan (SLRP) that has been shown to inhibit all three isoforms of TGFβ, namely TGFβ1, TGFβ2, and TGFβ3 with equal efficiency (Yamaguchi Y, et al. Nature 1990; 19:346:281-4; Border W A, et al. Nature 1992; 360:361-4; Mohan R R, et al. Curr Mol Med (In press). Structurally, decorin, like other proteoglycans, contains a core protein covalently bound to a glycosaminoglycan (GAG) side (Handley C J, et al. Adv Pharmacol 2006; 53: 219-32.).

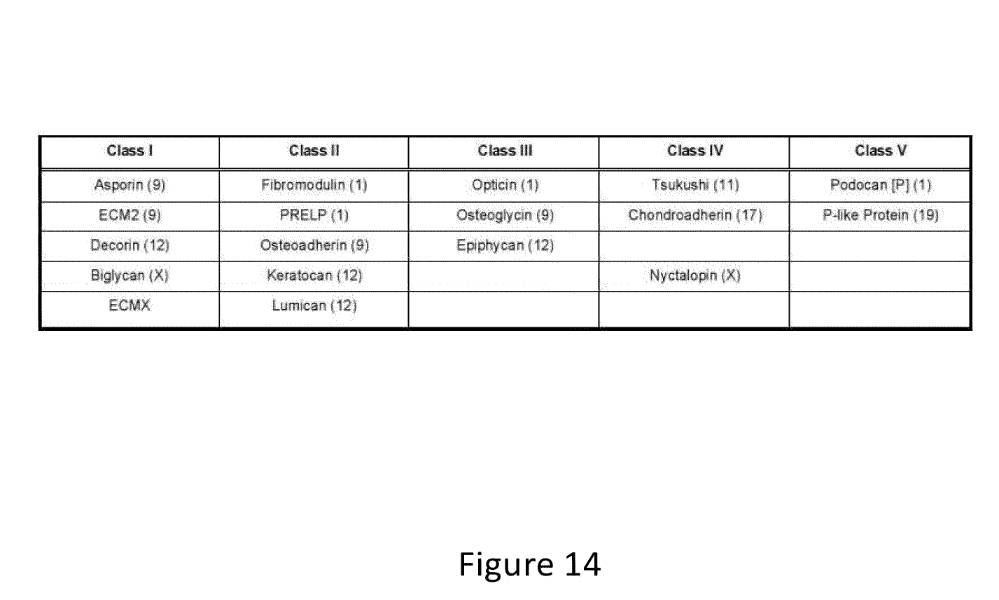

The SLRP gene family includes 17 genes that encode proteoglycans grouped into five distinct classes (FIG. 14) (Schaefer L and Iozzo R V. J Biol Chem 2008; 283: 21305-09.). This classification is based not only on common function but also common structure taking into account conservation and similarity at the gene and protein levels, occurrence of the typical amino-terminal cysteine-rich region with specific spacing, and chromosomal arrangement (Schaefer L and Iozzo R V. J Biol Chem 2008; 283: 21305-09.). Class I SLRPs include decorin and biglycan which share almost 60% homology and are the only SLRPs with a propeptide thought to be involved in enzyme signal recognition as part of GAG chain synthesis and conserved across species (Iozzo R V, et al. J Biol Chem 1999; 274: 4489-92.). Asporin and ECM protein 2 are other class I members. Class II boasts five members: fibromodulin, lumican, proline/argininerich end leucine-rich repeat protein or PRELP, keratocan, and osteoadherin. Class II SLRPs contain clusters of tyrosine sulfate residues at the N-terminal that may contribute to their polyanionic nature. Class III consists of three members (epiphycan, opticin, and osteoglycan) each with a low number of leucine-rich repeats. Class IV and V SLRPs are considered relatively new and non-canonical. Class IV is made up of 3 members (chondroadherin, nyctalopin, and tsukushi) while Class V is comprised of 2 members (podocan and the highly homologous podocan-like protein 1) (Schaefer L and Iozzo R V. J Biol Chem 2008; 283: 21305-09.).

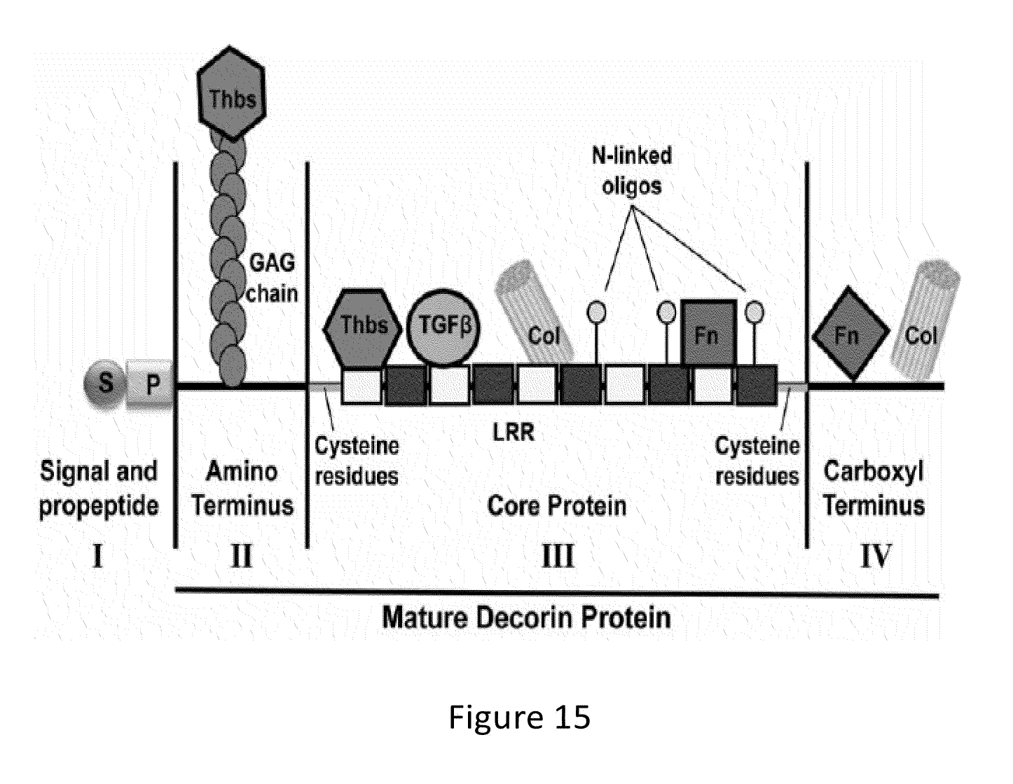

The five classes of ECM molecules that belong to the SLRP family share a common basic structure, namely a globular protein core linked to various GAG side chains. SLRPs contain a protein center made up of leucine-rich repeats (thus the acronym) responsible for their curved horseshoe-like structure advantageous for interactions with other proteins such as cytokines, growth factors and their receptors, and other ECM components (Kresse H and Schonherr E. J Cell Physiol 2001; 189: 266-74; Handley C J, et al. Adv Pharmacol 2006; 53: 219-32; Weber I T, et al. J Biol Chem 1996; 271: 31767-70.). In humans, the central part of the core protein of decorin (also known as decoron) is made up of 10-12 repeating leucine-rich motifs made up of 21-26 residues (Seidler DG and Dreier R. IUBMB Life 2008; 60: 729-33; Iozzo R V, et al. J Biol Chem 1999; 274: 4489-92; Scott P G, et al. Proc Natl Acad Sci USA 2004; 101: 15633-8; Roughley P J. Eur Cell Mater 2006; 12: 92-101.). The protein core is bordered on either side by conserved cysteine-rich clusters arranged into loops and stabilized by disulfide bonds (FIG. 15). Farther out from the cysteine residues, the GAG chain covalently attaches to decorin's protein core via a serine residue at the fourth amino acid position from the amino terminus (Chopra R K, et al. Biochem J 1985; 232: 77.). Decorin and biglycan, prototype members of the SLRP family, are substituted at the N-terminal with one or two chondroitin sulfate or dermatan sulfate side GAG chains respectively (Iozzo R V, et al. J Biol Chem 1999; 274: 4489-92; Fisher L W, et al. J Biol Chem 1989; 264: 4571-76.). Recently another salient feature of SLRPs, the C-terminal-specific ear repeat, was described by McEwan and cohorts [41]. SLRPs in classes I-III contain the repeat while those in the noncanonical classes do not. As such, some researchers hold that the ear repeat is the characteristic hallmark of true SLRP family members (McEwan P A, et al. J Struct Biol 2006; 155: 294-305.). Although the proposition stands structurally, it crumbles when SLRPs are examined functionally seeing that proteoglycans in class IV and class V share roles with members of first three classes (Schaefer L and Iozzo R V. J Biol Chem 2008; 283: 21305-09.).

As the archetypal SLRP, decorin was the first SLRP to have its protein core structure described in detail. This brought to light numerous associations concerning the SLRP family. First, SLRP molecules have related internal repeat structures; second, SLRPs have much less curvature than suggested by an earlier model based on decorin's three-dimensional structure; third, the cysteine-rich regions bordering the inner amino and carboxyl-terminals are conserved motifs that act as caps; and fourth, decorin, through the concave portion of its leucine-rich repeat domain, has the ability to dimerize. This has implications on other SLRPs in view of the fact that they may share a common mode of dimerization and overlap functionally (McEwan P A, et al. J Struct Biol 2006; 155: 294-305.). Although dimerization of decorin has been demonstrated in vitro, decorin likely functions as a monomer in vivo and may undergo a dimer-monomer transition allowing for the exposure of specific binding sites for proteins such as collagen (Schaefer L and Iozzo R V. J Biol Chem 2008; 283: 21305-09; McEwan P A, et al. J Struct Biol 2006; 155: 294-305; Orgel J P, et al. PLoS One 2009; 4:e7028.).

In humans, the 8 exon-containing decorin gene is located at chromosome 12 (12q22) and encodes a 359 amino acid translation product with four domains (Stander M, et al. Cell Tissue Res 1999; 296: 221-27; Roughley P J. Eur Cell Mater 2006; 12: 92-101; Grover J, et al. J Biol Chem 1995; 270: 21942-49.). The mature decorin protein is made up of three functional domains, domains II, III, and IV (FIG. 15). Domain I, virtually absent in the mature decorin protein, contains the original 30 aa residues of the N-terminus made up of the propeptide (14 aa) and signal peptide (16 aa). The signal peptide directs the incipient core protein to the endoplasmic reticulum while the propeptide may be involved in GAG side chain attachment to domain II as mentioned earlier (Stander M, et al. Cell Tissue Res 1999; 296: 221-27.). The propeptide also holds regulatory functions as regional aa deletions lead to swifter secretory pathway transport and a shorter GAG chain (Fransson L A, et al. Matrix Biol 2000; 19: 367-76.).

Domain II comprises the amino terminus of the mature decorin protein where the 50 kDa GAG chain resides (Stander M, et al. Cell Tissue Res 1999; 296: 221-27.). SLRPs have also been classified according to their GAG chain composition into keratan sulfate, heparan sulfate, dermatan sulfate, and chondroitin sulfate macromolecules (Tanihara H, et al. Cornea 2002; 21: S62-9.). Decorin's GAG side chain, usually chondroitin/dermatan sulfate depending on the tissue, binds to other GAG chains in addition to core proteins and also possesses a binding site for thrombospondin (Stander M, et al. Cell Tissue Res 1999; 296: 221-27.). The presence of disaccharides uronic acid and N-acetylgalactosamine has also led to the term galactosaminoglycans when referring to these side chain polymers. These highly sulfated oligosaccharides are difficult to characterize structurally owing to their heterogeneity. The GAG chains differ by their size, extent of glucuronic acid epimerization to iduronic acid, and O-sulfation. Generally speaking, the GAG chains present in decorin are found at the cell surface and throughout the ECM and pericellular matrix where they have multiple structural functions (Seidler D G and Dreier R. IUBMB Life 2008; 60: 729-33.).

Domain III, the most important domain functionally, contains the 40 kDa decorin core protein that comprises 80% of the SLRP [46]. Decorin's leucine-rich repeats contain the sequence LXXLXLXXNXL (SEQ ID NO: 1), where L is for the most part leucine and X represents any amino acid (Roughley P J. Eur Cell Mater 2006; 12: 92-101.). Each leucine repeat has 24 amino acids and is usually made up of an a-helix and β-turn (Jarvelainen H, et al. Wound Repair Regen 2006; 14: 443-52.). Found in more than three score proteins in prokaryotes and eukaryotes, leucine-rich repeats seem to have implications in protein to protein interactions (Blaschke U K, et al. J Biol Chem 1996; 271: 30347-53.). In addition to these leucine repeats, sulfated tyrosine and phosphorylated serine residues are present in this region. The substitution of two or three N-linked asparagine-bound oligosaccharides, believed to function in retarding self-aggregation, is another characteristic of domain III in human decorin ((Stander M, et al. Cell Tissue Res 1999; 296: 221-27; Scott P G, et al. J Biol Chem 1993; 268: 11558-64.). Domain III also houses binding sites for different proteins including fibronectin (heparin-binding domain), thrombospondin, and TGFβ (Stander M, et al. Cell Tissue Res 1999; 296: 221-27.). Recently it was shown that a high-affinity collagen-binding site exists in the sixth leucine rich repeat of domain III (Kalamajski S, et al. J Biol Chem 2007; 282: 16062-67.).

Domain IV, the carboxyl terminus domain, harbors an additional binding site for collagen, albeit with low affinity, as well as a site for the attachment of fibronectin's cell-binding domain (Stander M, et al. Cell Tissue Res 1999; 296: 221-27.). The general tertiary structure of decorin, vital in its ability to bind various molecules including collagen, is in the form of an arch or horseshoe (Jarvelainen H, et al. Pharmacol Rev 2009; 61: 198-223; Scott J E. Biochemistry 1996; 35: 8795-99.). Rabbit (NM_{—}001082330.1; SEQ ID NO: 4), human (NM_{—}001920; SEQ ID NO: 5), and mouse (NM_{—}001190451; SEQ ID NO: 6) decorin gene sequences are provided in the accompanying Sequence Listing.

### EXAMPLES

The following disclosed embodiments are merely representative of the invention which may be embodied in various forms. Thus, specific structural, functional, and procedural details disclosed in the following examples are not to be interpreted as limiting.

### Example 1

### Material and Methods

In Vivo and Ex Vivo Model:

Six to eight week old female C57 mice (18-21 gms) and New Zealand White rabbits (2.5-3.0 kg) were used for in vivo studies. The donor human corneas procured from eye banks were used for ex vivo investigations. All animals and human corneas were treated in accordance with the tenets of the ARVO Statement for the Use of Animals in Ophthalmic and Vision Research and the declaration of Helsinki. Mice were anaesthetized with intramuscular injection of ketamine (130 mg/kg) and xylazine (8.8 mg/kg) whereas rabbits were anaesthetized by intramuscular injection of ketamine hydrochloride (50 mg/kg) and xylazine hydrochloride (10 mg/kg). Topical ophthalmic 1% proparacaine hydrochloride solution (Alcon, Ft. Worth, Tex.) was instilled in each eye for local anesthesia.

Topical Drying and Vector Delivery Technique:

The corneal epithelium of the mouse and rabbit corneas was removed by gentle scraping with a #64 Beaver blade (Becton-Dickinson, Franklin Lakes, N.J.) under an operating microscope under general and local anesthesia. The epithelium of human cornea was removed similarly after placing tissue on the dried surface of the culture dish. After removing corneal epithelium eyes were washed with BSS (Alcon, Ft. Worth, Tex.) and wiped with a merocel sponge. The Conair hair dryer of 234 Watts (Model 1875; Stamford, Conn.) was used for drying the rodent, rabbit and human corneas. The temperature and air-flow of warm air were 410 C and 6.8 meter/second, respectively, according to digital Velocicheck anemometer (Model 8330; TSI Inc, Shoreview, Minn.). The hair dryer was operated from a distance of 8 inches and approximately 45° angle to the eye. The corneas either received no warm air (control) or warm air once for 10 seconds, twice for 10 seconds with 5 seconds interval, or thrice for 10 seconds with 5 seconds interval after every round or five times for 10 seconds with 5 seconds interval after every round. Immediately after drying, 2 μl BSS or vector was topically applied on the mouse cornea and 50 μl on the human and rabbit corneas for two minutes using a custom-cloning cylinder. The cloning cylinder of 3 mm diameter was used for the mouse and 7 mm diameter for the rabbit and human corneas.

Quantification of Vector Absorption:

Hamilton microsyringes (Reno, Nev.) and Gilson pipetman (0.2-2 μl) were used to dispense and quantify unabsorbed BSS or vector topically applied on the cornea using a cloning cylinder. After 2 minutes, all unabsorbed BSS/vector volume of the total 2 μl BSS/vector applied on the mouse cornea (n=12) or 50 μl BSS applied on the rabbit (n=12) and human (n=12) corneas was collected and measured. The amount of vector absorbed by the animal and human corneas was calculated by subtracting unabsorbed BSS/vector volume from the total applied BSS/vector volume. The results are expressed in percent.

AAV Vector Production:

AAV8 vector was generated using adenovirus free system following methods reported previously (Sharma A, Tovey J C, Ghosh A, Mohan R R. Exp Eye Res 2010; 91:440-8). Briefly, human embryonic kidney (HEK) 293 cells were co-transfected with AAV2-based genomic vector pARAP4, AAV8 Rep/Cap plasmid and adenovirus helper plasmid in a ratio of 1:3:3. The pARAP4 expresses heat stable placental alkaline phosphatase (AP) under the regulation of Rous sarcoma virus (RSV) promoter/enhancer and simian virus 40 (SV40) polyadenylation sequence. The virus containing cell lysate was harvested at 62 h post-transfection. Recombinant viral stocks were purified by two sequential rounds of CsCl gradient ultracentrifugation. Collected viral fractions were pooled and dialyzed through two rounds of HEPES-buffered saline. Viral titer was determined by dot blot analysis using DIG labeled probes (Roche Applied Science, Indianapolis, Ind.). The AAV genomic plasmid (pARAP4) was obtained from Dr. Dusty Miller, Fred Hutchison Cancer Research Center, Seattle Wash. and pAAV2/8 plasmid was procured from Dr. James M. Wilson, Gene Therapy Program, Division of Medical Genetics, University of Pennsylvania, Philadelphia Pa.

AAV8 Application to Mouse Cornea:

Thirty-six female C57 mice were used to study the effects of drying the levels of gene transfer. The study was approved by the Animal Care and Use Committees of the University of Missouri-Columbia and Harry S. Truman Memorial Veterans' Hospital Columbia, Mo. Mice were given general anesthesia with intramuscular injection of ketamine and xylazine and local anesthesia by instilling 1% proparacaine hydrochloride on the eye. After removing epithelium, 2 μl BSS or AAV8 (1.1×10⁸ genomic copies/μl) was topically applied on mouse cornea for 2 minutes using a custom-cloning cylinder (3 mm in diameter) as described earlier under vector delivery technique. The mice were divided into 5 groups: Group-1 corneas received warm air for 10 seconds, Group-2 corneas received 2 rounds of warm air for 10 seconds with 5 seconds interval, Group-3 received 3 rounds of warm air for 10 seconds with 5 seconds interval after every round, Group-4 received 5 rounds of warm air for 10 seconds with 5 seconds interval after every round and Group-5 corneas did not receive any warm air after merocel wiping and served as a control. All animals were sacrificed at 14 days after BSS/vector application.

Tissue Embedding:

Mouse eyes were enucleated and embedded in liquid OCT compound (Sakura FineTek, Torrance, Calif.) within a 15 mm×15 mm×5 mm mold (Fisher, Pittsburgh, Pa.) and snap frozen as reported previously (Sharma A, Tovey J C, Ghosh A, Mohan R R. Exp Eye Res 2010; 91:440-8). The frozen tissue blocks were maintained at −80° C. Seven micron thick tissue sections were cut with a cryostat (HM 525M, Microm GmbH, Walldorf, Germany) and maintained frozen at −80° C. until staining.

Tissue Morphology and Gene Delivery:

Corneal tissue morphology was analyzed with hematoxylin and eosin (Fisher Scientific) staining following vendor's protocol. The delivered marker alkaline phosphatase (AP) gene expression was determined with cytochemical staining following manufacturer's instructions. In brief, tissue sections were washed with HEPES buffer and incubated with a mixture of 5-bromo-4-chloro-3′-indolylphosphate p-toluidine (BCIP) and nitro-blue tetrazolium (NBT) at 37° C. The AP-stained corneal stroma appeared dark blue. Gene transfer was quantified by determining the pixels of AP stained area in 400× magnification using National Institutes of Health Image J 1.38× (NIH, Bethesda, Md.) software.

Inflammatory Response:

The effects of corneal drying on inflammatory reaction were analyzed by the CD11b and F4/80 immunocytochemistry. The immunofluorescence staining for CD11b (BD Pharmingen, San Jose, Calif.) and F4/80 (Serotec, Raleigh, N.C.) was performed using rat anti-mouse antibodies. Tissue sections (7 μm) were washed with 1×HEPES, blocked in 5% BSA for 30 min. followed by incubation at room temperature with the primary antibody at 1:50 dilution for 90 min and with secondary antibody goat anti-rat IgG (AlexaFlour 594, Molecular Probes, Eugene, Oreg.) at a dilution of 1:500 for 60 min. Vectashield mounting medium containing DAPI (Vector Laboratories, Inc. Burlingame, Calif.) was used to visualize nuclei in the tissue sections. The sections were viewed and photographed under a Leica fluorescent microscope (Leica, Wetzlar, Germany) equipped with a digital camera.

Statistical Analysis:

The results were expressed as mean±standard error of the mean (SEM). Statistical analysis was performed using either one-way analysis of variance (ANOVA) followed by Tukey's multiple comparison test or two-way ANOVA followed by Bonferroni test.

### Results

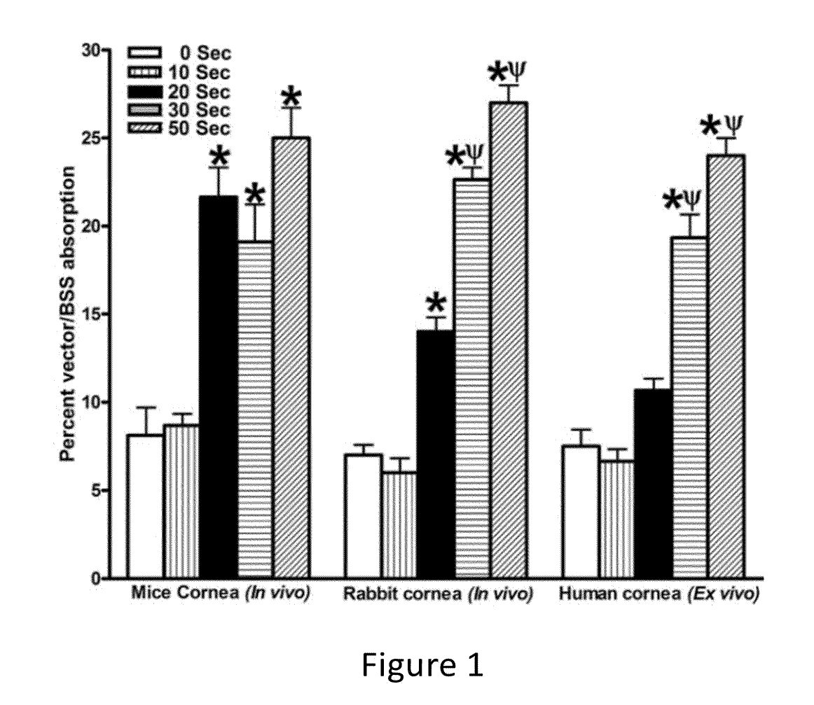

FIG. 1 shows the effect of corneal drying on absorption of topically applied BSS solution in mouse, rabbit, and human corneas and provides evidence that the uptake of topical solution is significantly enhanced after corneal drying. As shown in FIG. 1, mouse corneas subjected to 0 seconds or 10 seconds of drying showed 8±1.5% absorption of topically applied BSS solution. Drying for 20 seconds, 30 seconds, or 50 seconds significantly (p<0.001) enhanced the absorption to 21±1.4%, 19±2.1%, and 25±1.5%, respectively.

Zero and 10 seconds drying of rabbit corneas resulted in 7±1.1% absorption of topically applied BSS solution. Twenty seconds of warm air drying significantly (p<0.001) enhanced corneal absorption to 14±0.8%. Thirty and 50 seconds of warm air drying in rabbits resulted in further increase in corneal absorption to 22±0.6% and 27±0.8% (p<0.001) (FIG. 1).

Zero and 10 seconds of drying of human corneas resulted in 7±0.6% absorption of topically applied BSS solution. Twenty seconds of warm air drying enhanced corneal absorption to 10±0.6% (not statistically significant). Human corneas subjected to 30 seconds or 50 seconds of drying showed 19±1.15% and 24±0.9% absorption, respectively, which was significantly more (p<0.001) compared to corneas subjected to either zero, 10, or 20 seconds of air drying (FIG. 1).

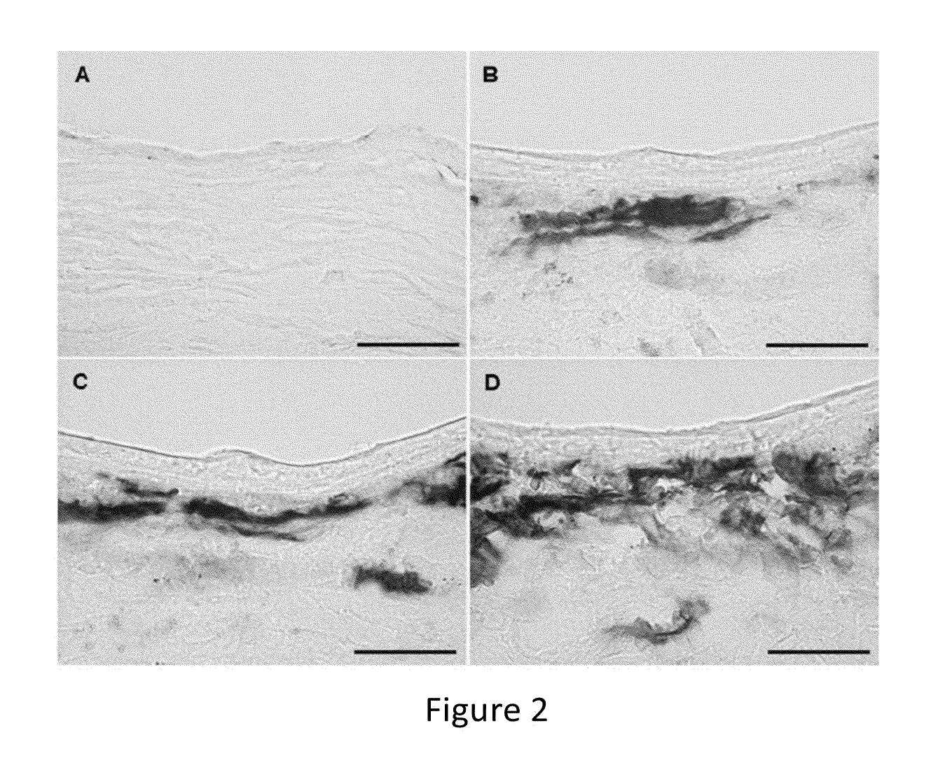

FIGS. 2A to 2D show the effect of different rounds of corneal drying on AAV-mediate gene expression. Corneas exposed to 20 seconds or less of drying showed mild to moderate levels of gene expression (FIGS. 2A and 2B). Corneas exposed to 30 seconds of drying demonstrated significantly more gene expression, while 50 seconds of drying resulted in further enhancement of gene expression (FIGS. 2C and 2D).

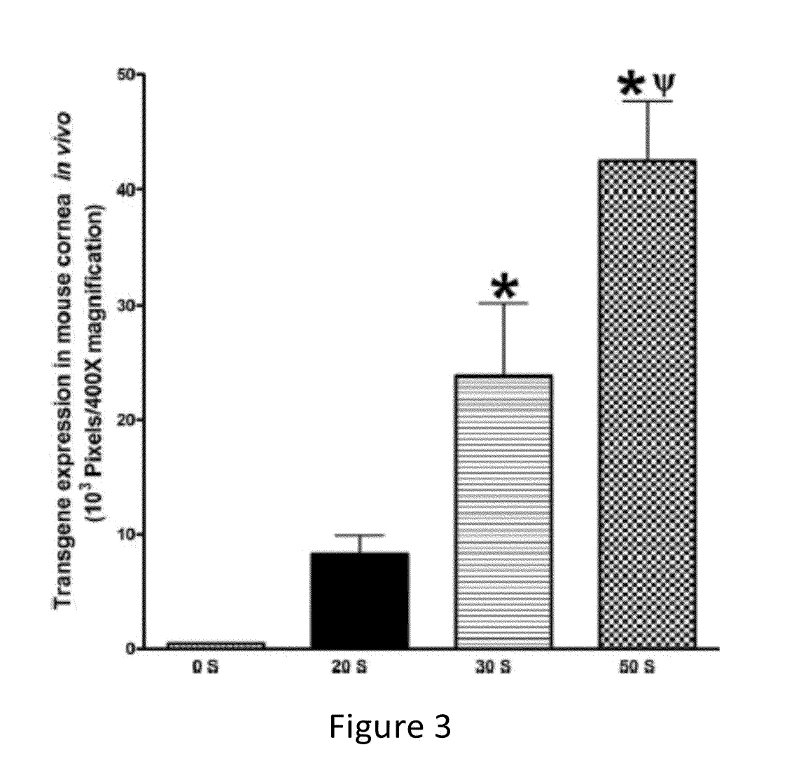

FIG. 3 shows the quantification of gene expression in corneas subjected to 20, 30, or 50 seconds of warm air drying. Corneas exposed to 50 seconds of drying showed the highest level of gene expression and it was significantly (p<0.05) more compared to groups subjected to 20 seconds and 30 seconds of drying. Relative comparison between the 30 seconds and 20 seconds treatment group revealed significantly (p<0.05) higher levels of gene expression in corneas exposed to 30 seconds of drying.

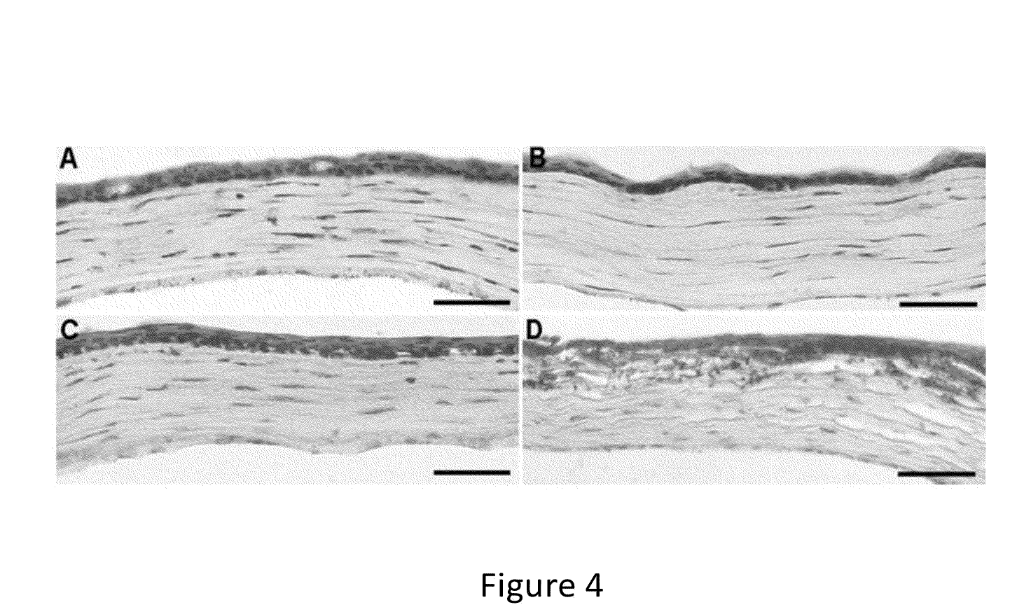

FIGS. 4A to 4D show hematoxylin and eosin-stained corneal sections obtained from mice subjected to zero seconds, 20 seconds, 30 seconds, or 50 seconds of warm air drying. No apparent structural abnormalities were detected in mouse corneas subjected to 30 or less seconds of warm air drying. On the other hand, mouse corneas exposed to 50 seconds of warm air drying shoed structural damage in the anterior stroma. A large number of hematoxylin-stained nuclei were also detected in the anterior stroma of these corneas, suggesting the possibilities of inflammatory cells.

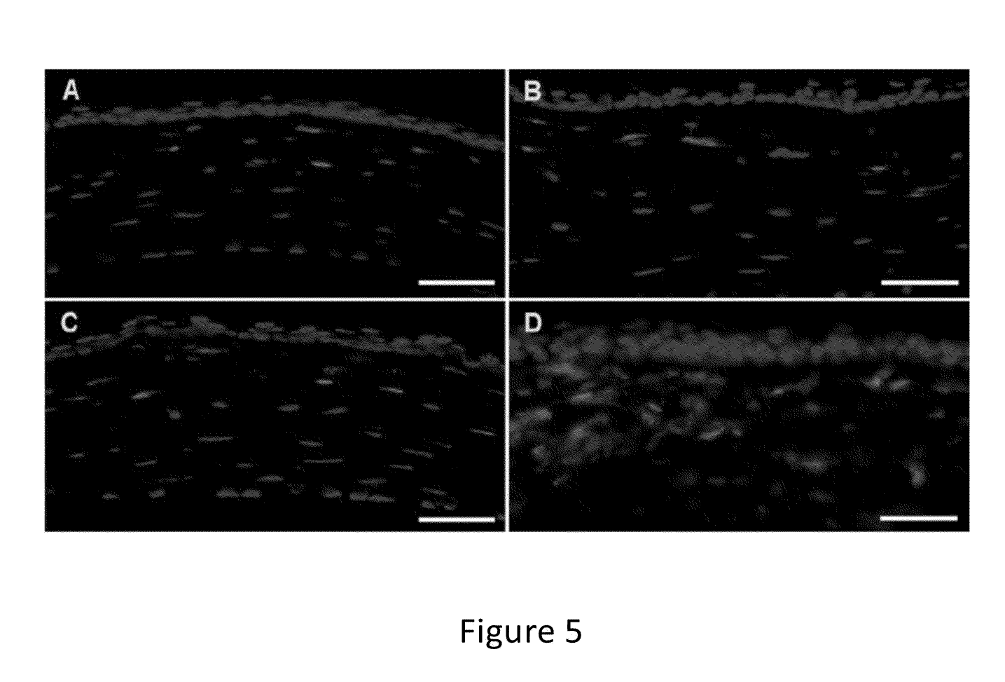

The presence of inflammatory cells was confirmed by immunostaining for CD11b, a marker for activated granulocytes, and F4/80, a macrophage specific antigen. FIGS. 5A to 5D show CD11b staining in corneas exposed to zero seconds, 20 seconds, 30 seconds, or 50 seconds of warm air drying. Mouse corneas subjected to 30 seconds of warm air showed 7±2 CD11b+ cells and 50 seconds showed 33±5 CD11b+ cells, suggesting increase infiltration of activated granulocytes in severely dried cornea.

It was also observed that application of five rounds of ten seconds air drying comprised corneal morphology in vivo and three rounds of ten seconds drying significantly augmented gene transfer without jeopardizing corneal morphology and one or two rounds of ten second drying augmented only a mild-to-moderate degree of gene transfer with no altered corneal morphology.

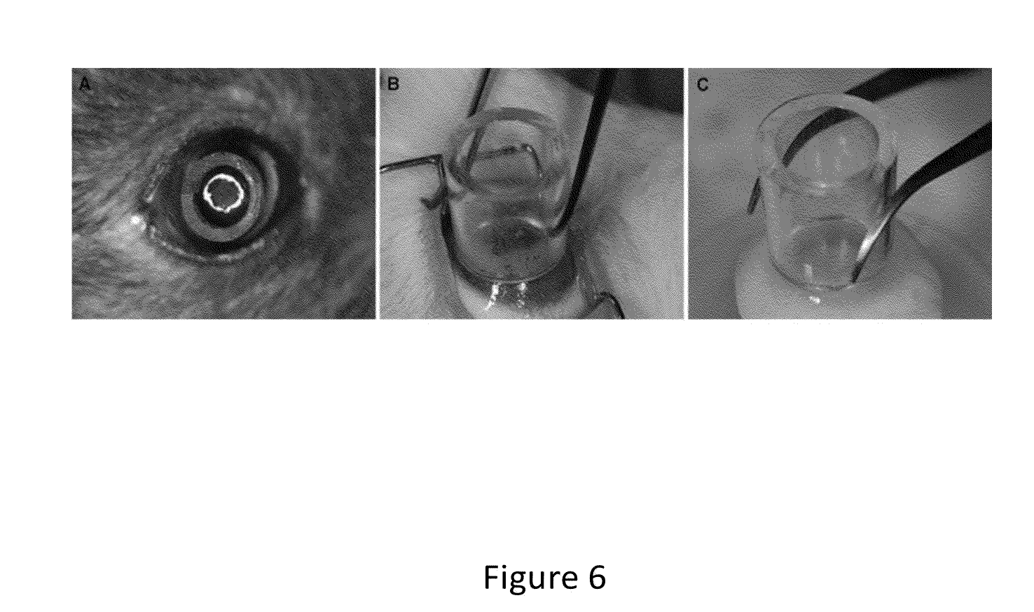

FIGS. 6A to 6C show the localized deliveries (among three species) within a cloning cylinder in vivo. Dispensing of the viral vector inside the physical barrier limits contact of the viral vector to neighboring ocular tissue (FIGS. 6A, 6B, and 6C). This significantly enhances transgene delivery into the targeted corneal stroma and prevents transgene delivery into untargeted tissues such as limbus, conjunctiva, sclera, etc.

### Example 2

A well-established laser-based experimental rabbit corneal scarring method was used to demonstrate proof of concept. The model cornea stroma was treated with decorin gene via tissue-selective targeted gene delivery. Decorin-treatment was evaluated based on biomicroscopic quantification, immunohistochemical determination, and immunoblot quantification of corneal fibrosis and found that tissue-selective targeted decorin gene delivery in the cornea with AAV5 significantly retards corneal fibrosis in vivo.

### Materials and Methods

Animals

Twenty-four female New Zealand White rabbits (Myrtle laboratories Inc., Thompson's Station, Tenn.) weighing 2.5-3.0 kg were used in this study. The Institutional Animal Care and Use Committee of the University of Missouri-Columbia and Harry S. Truman Memorial Veterans' Hospital Columbia Mo. approved the study. All animals were treated in accordance with the Association of Research for Vision and Ophthalmology Statement for the Use of Animals in Ophthalmic and Vision Research. Rabbits were anesthetized by intramuscular injection of a mixture of ketamine hydrochloride (50 mg/kg) and xylazine hydrochloride (10 mg/kg). In addition, topical ophthalmic 0.5% proparacaine hydrochloride eye (Alcon, Fort Worth, Tex.) was used for local anesthesia.

Corneal Fibrosis in Rabbit Eves

A well-established PRK technique was used to produce fibrosis in the rabbit cornea (Sharma A, Mehan M M, Sinha S, Cowden J W, Mohan R R. Trichostatin A inhibits corneal haze in vitro and in vivo. Invest Ophthalmol V is Sci. 2009; 50:2695-2701). In all 24 rabbits, corneal haze was induced only in one eye and contralateral eye served as a naive or PRK-untreated control. In an anesthetized rabbit 2-3 drops of proparacaine hydrochloride solution were instilled to cornea and a wire lid speculum was inserted to expose the corneal surface. The corneal epithelium was removed by gentle scraping with a #64 Beaver blade (Becton-Dickinson, Franklin Lakes, N.J.) and PRK was performed by creating a 6 mm ablation zone to −9 diopters with the excimer laser (Summit Apex; Alcon) to produce fibrosis in the cornea as reported previously. This PRK technique has been shown to consistently produce fibrosis and myofibroblasts in the rabbit corneas that peaks at 4 weeks.

AAV-Decorin Plasmid Generation and Titer Production

Decorin gene was PCR-amplified from rabbit corneal fibroblast cDNA using forward (5′-GAT CGC GGC CGC AAT CAT GAO GGC AAC TCT CAT C-3′) (SEQ ID NO: 2) and reverse (5′-GTC AGC GGC CGC GAG TTA CTT GTA GTT TCC GAG C-3′) (SEQ ID NO: 3) primers. The amplified PCR product was cloned into AAV2 plasmid pTRUF11 containing a hybrid promoter (cytomegalovirus enhancer and chicken 11-actin) and simian virus 40 polyadenylation site using Not1 site. The resultant decorin cloned plasmid was packaged into AAV5 using 2-plasmid co-transfection method reported previously. In brief, approximately 1×109 HEK 293 cells were cultured in Dulbecco's Modified Eagle's Medium (Hyclone Laboratories, Inc. Logan, Utah, USA), supplemented with 5% fetal bovine serum and antibiotics. A CaPO4 transfection method was used by mixing equal molar ratio (1:1) of decorin cloned AAV2 plasmid and AAV5 rep-cap helper plasmid. This precipitate was applied to the cell monolayer and the transfection was allowed to incubate at 37° C., 7% CO₂ for 60 h. The cells were then harvested and lysed by freeze/thaw cycles and subjected to discontinuous iodixanol gradients centrifugation at 350,000 g for 1 h. This iodixanol fraction was further purified and concentrated by column chromatography on a 5-ml HiTrap Q Sepharose column using a Pharmacia AKTA FPLC system (Amersham Biosciences, Piscataway, N.J., USA). The vector was eluted from the column using 215 mM NaCl buffer, pH 8.0, and the rAAV peak collected. AAV5 decorin (AAV5-dcn) vector-containing fraction was then concentrated and buffer exchanged in Alcon BSS with 0.014% Tween 20, using a Biomax 100K concentrator (Millipore, Billerica, Mass., USA). Vector was titrated for DNAse-resistant vector genomes by Real-Time PCR relative to a standard.

AAV5 Transduction to Rabbit Cornea

The rabbits were divided into two groups. Group I corneas received 100 μl AAV5 titer (6.5×10¹² vg/ml) expressing decorin gene (n=12) topically for two minutes via a custom cloning cylinder technique immediately after PRK surgery. Group II corneas received 100 μl AAV5 titer (6.5×10¹² vg/ml) expressing green fluorescent protein gene (n=12). The contralateral naïve corneas served as PRK-untreated (n=12) and AAV5-untreated (n=12) negative controls. For each group, slitlamp biomicroscopy was performed in all 12 treated eyes before euthanasia 4 weeks after PRK and vector application. After euthanasia six corneal tissues were used for immunocytochemistry and microscopy, 2 for western-blotting, 2 for slot-blotting and 2 for transmission electron microscopy analyses.

Slitlamp Biomicroscopy in Live Rabbits

The level of corneal haze and health in the eyes of live rabbits was examined by visual clinical and slitlamp microscopic (BX 900 Slit Lamp, Haag-Streit-USA, Mason Ohio) examinations before PRK and 4 weeks after PRK as described earlier. Grade 0 was a completely clear cornea; grade 0.5 had trace haze seen with careful oblique illumination with slit lamp biomicroscopy; grade 1 was more prominent haze not interfering with the visibility of fine iris details; grade 2 was mild obscuration of iris details; grade 3 was moderate obscuration of the iris and lens; and grade 4 was complete opacification of the stroma in the area of ablation. Haze grading was performed in a masked manner. Optical coherence tomography was performed using Cirrus 3000 high-definition instrument (Carl Zeiss Meditec, Dublin, Calif.) in live rabbits under general anesthesia to analyze corneal thickness. The scans with the best signal strength were selected, and imaging data was analyzed with Cirrus optical coherence tomography system software (version 3.0; Carl Zeiss Meditec, Dublin, Calif.).

Euthanasia and Tissue Collection

Rabbits were humanely euthanized with pentobarbitone (150 mg/kg) overdose under general anesthesia 4 weeks after PRK and vector application. Corneas of six rabbits of each group were removed with forceps and sharp Westcott scissors, embedded in liquid optimal cutting temperature (OCT) compound (Sakura FineTek, Torrance, Calif.) within a 24 mm×24 mm×5 mm mold (Fisher Scientific, Pittsburgh, Pa.) and snap frozen as reported earlier. 34 Frozen tissue blocks were maintained at −80° C. for future use. Tissue sections were cut 7 or 20 μm thick with a cryostat (HM 525M, Microm GmbH, Walldorf, Germany), placed on 25 mm×75 mm×1 mm microscope Superfrost Plus slides (Fisher), and maintained frozen at −80° C. until staining. The remaining six rabbit corneal tissues of each group were immediately either frozen in liquid nitrogen for western blotting (n=2) and slot-blotting (n=2) or fixed in buffer for transmission electron microscopy (n=2).

Immunofluorescence Studies

Immunofluorescence staining for alpha smooth muscle actin (αSMA), a marker for myofibroblasts, was performed using mouse monoclonal primary αSMA antibody (1:200 dilution, catalog no. M0851, Dako, Carpinteria, Calif.). Tissue sections were incubated with 2% bovine serum albumin for 30 minutes at room temperature and then with αSMA monoclonal antibody for 90 minutes. For the detection of the primary antibody, Alexa 488 goat anti-mouse IgG secondary antibody (1:1000 dilution; catalog no. A11001, Invitrogen Inc., Carlsbad, Calif.) for 1 hour was used. SMA-positive cells in six randomly selected, nonoverlapping, full-thickness central corneal columns extending from the anterior stromal surface to the posterior stromal surface were counted according to a method reported previously. The diameter of each column was 400× magnification field.

Fibronectin immunostaining was carried out by incubating the tissue sections in goat polyclonal primary antibody (1:200 dilution; catalog no. sc6952; Santa Cruz Biotechnology Inc., Santa Cruz, Calif.) for 90 minutes. For primary antibody detection, Alexa 594 donkey anti-goat IgG secondary antibody (1:500 dilution; catalog no. A11058, Invitrogen) was used for 60 minutes. F-actin staining was performed using Alexa594-conjugated phallotoxin (A12381 Invitrogen). Tissues were incubated at 1:100 dilution for 90 minutes followed by subsequent washing with HEPES.

The possibility of immunological reaction to AAV5 mediated decorin gene therapy was examined by performing CD11b (catalog no. BDB550282 BD Pharmingen, San Jose, Calif.) and F4/80 (catalog no. MCA497 Serotec, Raleigh, N.C.) immunostaining in rabbit corneal sections using rat anti-mouse antibody. Tissue sections were incubated at room temperature with the CD11b primary antibody at a 1:50 dilution in a 1×HEPES buffer containing 5% BSA for 90 min, followed by goat anti-rat IgG secondary antibody (AlexaFlour 594, catalog no. A-11007, Invitrogen) at a 1:500 dilution for 60 min. After all of the above immunostaining, tissue sections were mounted with vectashield mounting medium containing DAPI (catalog no. H1200, Vector Laboratories, Inc. Burlingame, Calif.), viewed and photographed under a fluorescent microscope (Leica, Deerfield, Ill.) equipped with a digital SpotCam RT KE camera system (Diagnostic Instruments, Sterling, Mich.).

TUNEL Assay

TUNEL assay was performed in acetone fixed rabbit corneal sections using fluorescent apoptosis detection assay (ApopTag; catalog no. S7165 Millipore, Billerica, Mass.) that detects apoptosis and, to a lesser extent, necrosis following manufacturer's instructions. Positive control (4 hours after mechanical corneal scrape) and negative control (unwounded) were included in each assay.

Immunoblotting

Rabbit corneal tissues were lysed in RIPA protein lysis buffer containing protease inhibitor cocktail (catalog no. 11836153001 Roche Applied Sciences, Indianapolis, Ind.). Protein samples were prepared for electrophoresis by heating at 90° C. for 2 minutes followed by centrifugation at 10,000 g for 10 minutes. The samples were transferred onto polyvinylidene difluoride (PVDF) membranes (Invitrogen, San Diego, Calif.) using iBlot apparatus (Invitrogen, San Diego, Calif.), proteins were detected with the following primary antibodies: αSMA (mouse monoclonal, 1:200 dilution, catalog no. M0851, Dako) and R-actin primary antibody (catalog no. sc-69879; Santa Cruz Biotechnology Inc., Santa Cruz, Calif.) followed by alkaline phosphatase conjugated anti-mouse secondary antibody (catalog no. PR-53721 Fisher Scientific). After washing three times in 0.05% Tween-20 in TRIS-buffered saline pH 8.0 for 5 minutes each, the blot was developed using nitroblue tetrazolium (NBT)/5-bromo-4-chloro-3-indolylphosphate (BCIP) method.

Transmission Electron Microscopy

Rabbit corneas were fixed in a 2% glutaraldehyde and 2% paraformaldehyde solution in 0.1 M Na-cacodylate buffer (pH 7.4), post-fixed in 1% osmium tetroxide, sequentially dehydrated in ethanol and transitionally dehydrated in acetone, followed by infiltration with an Epon and Araldite (Electron Microscopy Sciences, Hatfield, Pa.) resin mixture. The embedded cornea samples were sectioned 85 nm thin using Leica Ultracut UCT ultramicrotome and transferred onto a 200 mesh copper grid for post-staining with uranyl acetate and Sato's triple lead stain. The samples were then imaged using JEOL 1400 transmission electron microscope (Tokyo, Japan).

Slot-Blotting to Determine Gene Copy Number

The copies of delivered plasmid were determined with slot blot analysis. The frozen corneal tissues were ground in liquid nitrogen and DNA was isolated using DNA easy kit (Qiagen, cat #69504). The standards were prepared using 104-1011 copies of decorin gene cloned into pTRUF11 vector. The DNA probe was prepared by digesting 5 μg of decorin plasmid with Not1 restriction enzyme and labeling 1 μg of isolated decorin fragment with digoxigenin (DIG)-labeled UTP, using DIG starter Kit II (catalog no. 11585614910 Roche Applied Science, Indianapolis, Ind.). Two microliters of the standard as well as the DNA isolated from corneal tissues was denatured by alkali and heat treatment. Denatured DNA samples were blotted onto nylon membrane using slot blot apparatus (BioRad lab) and were UV-cross linked. The membrane was hybridized with 300 ng of digoxigenin (DIG)-labeled probe overnight at 30° C., followed by incubation in 1:5000 anti-digoxigenin-AP antibody. Chemiluminescent detection was used following vendor's instructions (catalog no. 11585614910 Roche Applied Science, Indianapolis, Ind.) and membranes were exposed to X-ray film. Image J 1.38× image analysis software was used to determine delivered gene copies in samples by measuring dot intensities of samples and comparing the data with standards.

Statistical Analyses

Results of corneal haze grading, SMA quantification and collagen fibril diameter were expressed as mean±standard error mean. Statistical analysis was performed using student t-test or Wilcoxon rank sum test. The value of p<0.05 was considered significant.

### Results

Biomicroscopic Quantification of Corneal Fibrosis.

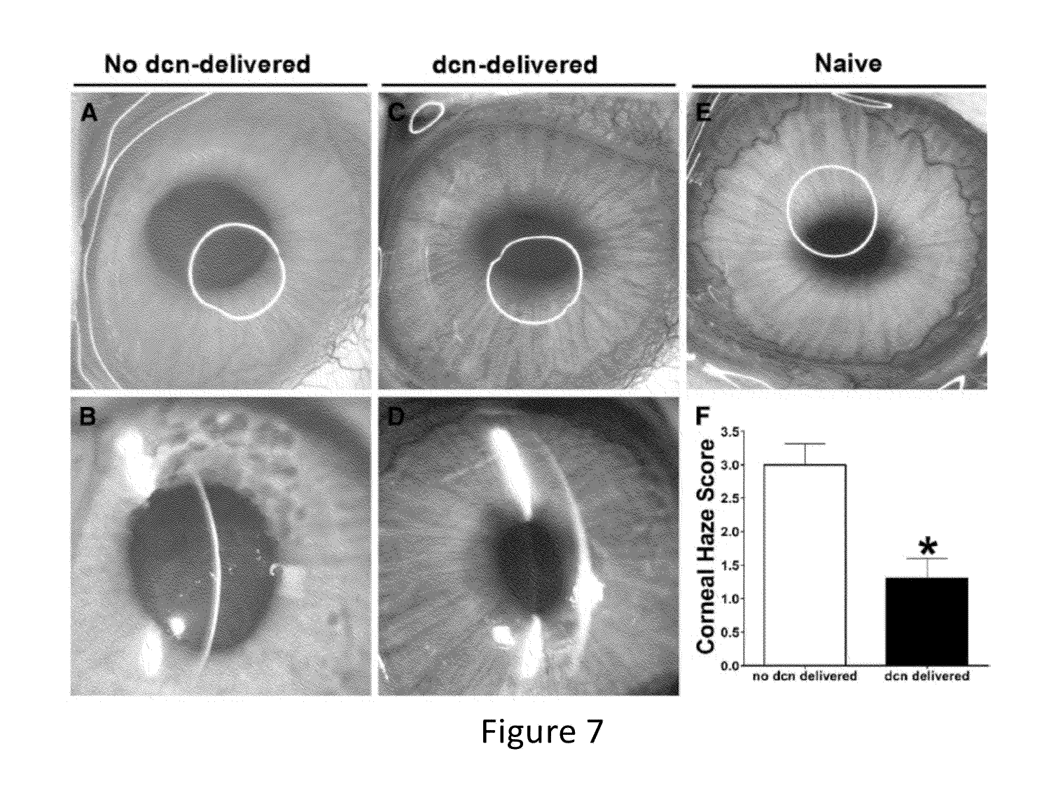

FIGS. 1A to 1F show slitlamp- and stereo-biomicroscopy images depicting the corneal haze in no-decorin-delivered (control) and decorin-delivered rabbit corneas. The PRK-treated rabbit eyes that received AAV5-gfp vector showed a strong fibrotic response in the cornea as evident from the cloudiness (FIGS. 7A and 7B). The PRK-treated rabbit eyes that received AAV5-dcn vector showed a substantial decrease in corneal cloudiness (FIGS. 7C and 7D). The PRK- or AAV5-untreated negative control rabbit corneas showed no corneal haze (FIG. 1E). The quantification of haze inhibition after decorin gene therapy was performed in a blinded fashion by three researchers. FIG. 7F shows mean haze score in decorin-delivered and no-decorin-delivered rabbit corneas observed 4 weeks after vector application. The no-decorin-delivered rabbit corneas showed an average haze score of 3.0±0.4, whereas decorin-delivered rabbit corneas showed significantly (p<0.01) lower corneal haze score of 1.3±0.3. Optical coherence tomography did not detect any significant difference in corneal thickness among the naïve, no-decorin-delivered, and decorin-delivered rabbit corneas. These finding suggest that AAV5-mediated decorin gene therapy is highly efficient in preventing corneal fibrosis and appears safe for the rabbit cornea.

Immunohistochemical Determination of Corneal Fibrosis.

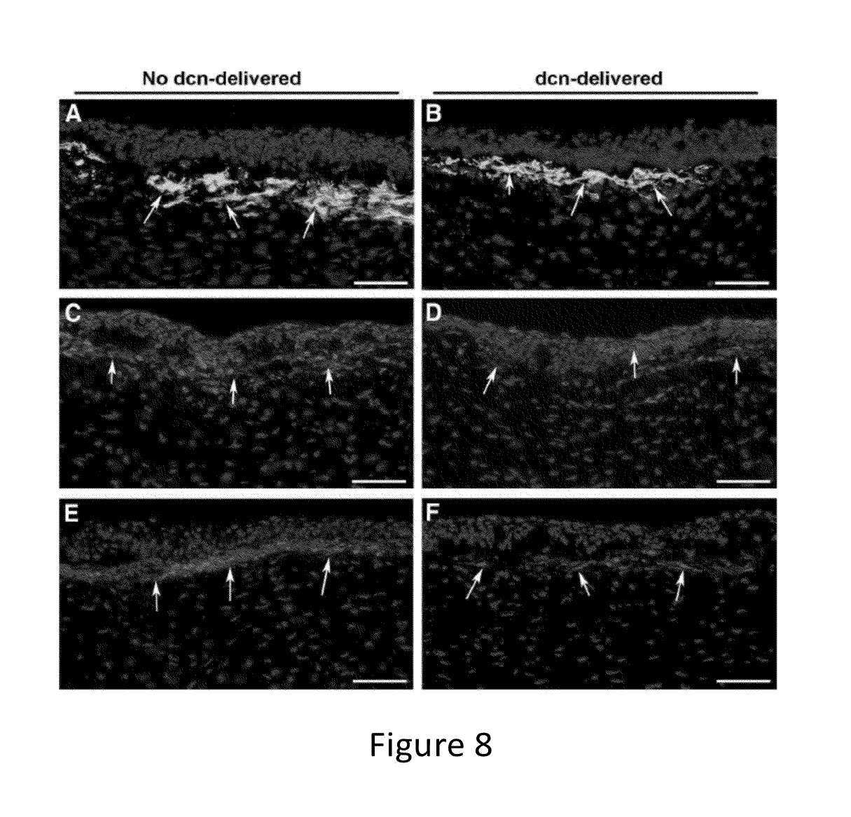

FIGS. 8A to 8F show the immunostaining for αSMA (FIGS. 8A and 8B), f-actin (FIGS. 8C and 8D), and fibronectin (FIGS. 1E and 1F) in decorin-delivered and no-decorin-delivered rabbit corneas. Myofibroblast formation is a hallmark of corneal fibrosis and is characterized by the expression of cytoskeletal proteins such as αSMA, f-actin, and fibronectin. The inhibitory effects of decorin gene delivery on corneal fibrosis were evaluated by immunostaining rabbit corneal tissues for αSMA, f-actin, and fibronectin. As shown in FIG. 8A, the rabbit corneas subjected to PRK without AAV5-dcn vector treatment showed intense αSMA immunostaining in the anterior stroma confirming the presence of myofibroblasts and the development of haze. Conversely, decorin-delivered rabbit corneas showed a significant decrease in αSMA expression (FIG. 8B) suggesting that AAV5-mediated decorin gene therapy effectively attenuates fibrosis in the rabbit cornea in vivo. The anti-fibrotic effects of decorin gene therapy were revalidated by f-actin (FIGS. 8C and 8D) and fibronectin (FIGS. 8E and 8F) immunocytochemistry. Both, additional tested fibrotic parameters showed similar levels of decrease as noted for SMA, providing further support that AAV5-delivered decorin in the stroma modulates ECM proteins, would healing and inhibits scar formation in the rabbit cornea. Phallodin staining also detected the filamentous actin of the epithelial borders (FIGS. 8C and 8D), that used anti-mouse monoclonal SMA antibody was able to recognize feebly (FIGS. 8A and 8B). The naïve rabbit corneas did not show any positive staining of tested antigens in the stroma.

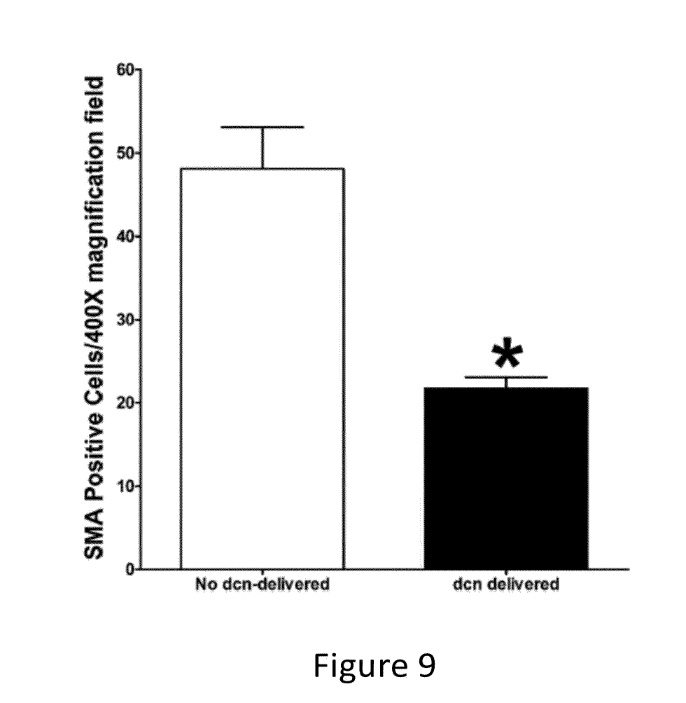

FIG. 9 shows the quantification of αSMA immunostaining in the rabbit corneas subjected to PRK with AAV5-dcn or AAV5-gfp treatment. As evident from the data, AAV5-mediated decorin gene therapy given by a single 2-minutes topical application caused a statistically significant decrease in αSMA (59%; p<0.001). The digital quantification of f-actin and fibronectin immunocytochemistry data showed significant decrease in f-actin (72%; p<0.001) and fibronectin (64%; p<0.01) expression in the rabbit cornea in vivo.

Immunoblotting Quantification of Corneal Fibrosis.

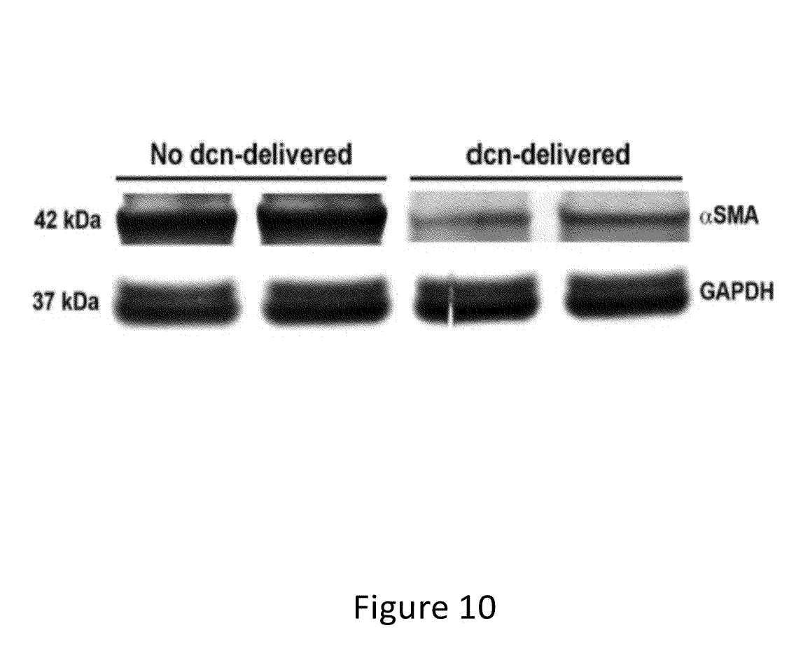

FIG. 10 shows the efficacy of AAV5-mediated decorin gene therapy on corneal fibrosis with αSMA immunoblotting. FIG. 10 shows results of αSMA western blotting performed with protein lysates of the decorin-delivered and no-decorin-delivered rabbit corneas collected 4 weeks after PRK (haze is at its peak at this point). The quantification of immunoblotting data with Image-J demonstrated a statistically significant reduction in the expression of αSMA in decorin-delivered corneal samples (67%; p<0.01) compared to no-decorin-delivered control corneal samples. The same intensity β-actin band in each lane confirmed equal loading of protein samples. This data further indicates that AAV5-mediated decorin gene therapy is significantly effective in reducing corneal fibrosis in a rabbit model.

Safety and toxicity of AAV5-mediated decorin gene therapy was evaluated. The CD11b (activated granulocytes marker) and F4/80 (macrophage marker) immunostaining and TUNEL assay were used to analyze the immunogenicity and cytotoxicity of the AAV5 vector for corneal decorin gene therapy.

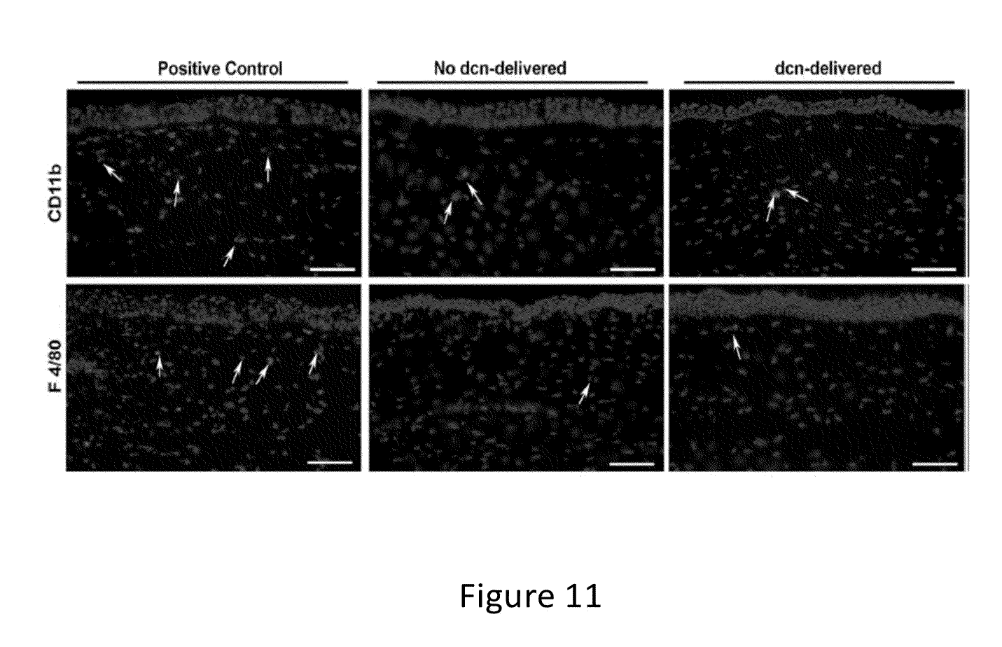

FIG. 11 shows the effects of AAV5-decorin gene therapy on immune response in rabbit corneas using CD11b and F4/80 immunocytochemistry. Anti-mouse CD11b and F4/80 antibodies known to cross-react with rabbit antigen were used (Barton R W, Rothlein R, Ksiazek J, Kennedy C. The effect of anti-intercellular adhesion molecule-1 on phorbol-ester-induced rabbit lung inflammation. J. Immunol. 1989; 143:1278-82); (Rogers C, Edelman E R, Simon D I. A mAb to the beta2-leukocyte integrin Mac-1 (CD11b/CD18) reduces intimal thickening after angioplasty or stent implantation in rabbits. Proc Natl Acad Sci USA. 1998; 95:10134-9). Furthermore, to insure their reactivity to the rabbit corneal tissues, alkali burn rabbit corneas collected 4 hours after 30 seconds of 1M sodium hydroxide application on the cornea were used as a positive control. FIG. 11 shows the results of CD11b and F4/80 immunostaining detected in the alkali-burn (left panels), no-decorin-delivered (middle panels), and decorin-delivered (right panels) rabbit corneas. The positive control alkali-burn corneal sections showed numerous CD11b+ and F4/80+ cells confirming that used antibodies recognized rabbit antigens. The naïve, no-decorin-delivered (FIG. 11, middle panels), and decorin-delivered (FIG. 11, right panels) rabbit corneal sections showed 2-6 CD11b+ or F4/80+ cells. The detection of occasional and statistically insignificant CD11b+ or F4/80+ cells in the naïve, no-decorin-delivered, and decorin-delivered rabbit corneas suggest that AAV5-delivered decorin gene therapy does not induce significant immune response in the cornea.

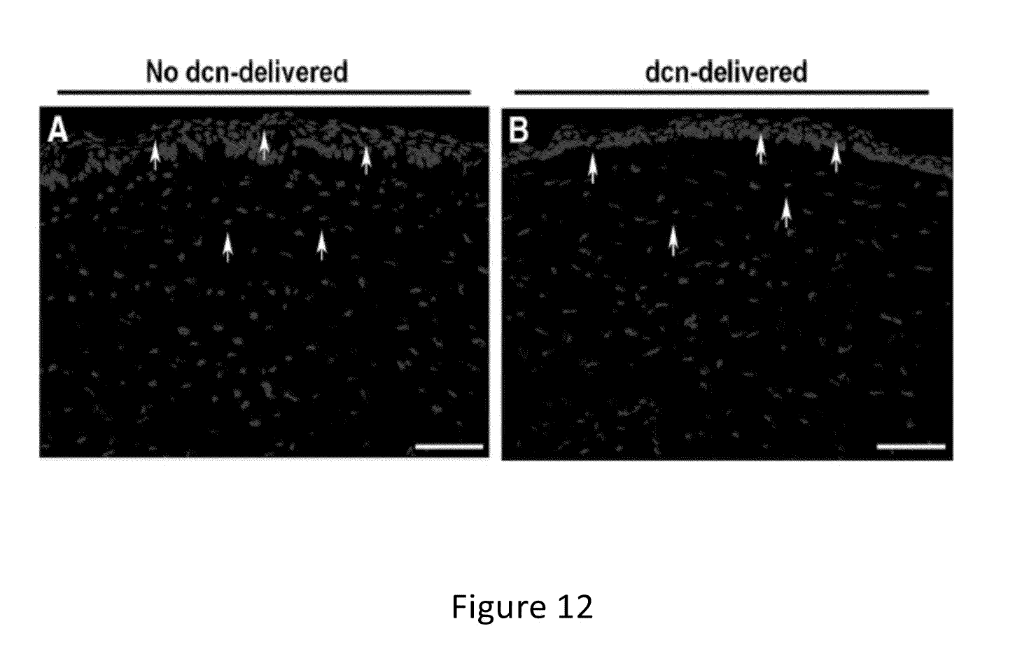

FIGS. 12A and 12B show analysis of in vivo cytotoxicity of AAV5-mediated decorin gene therapy for the corneas by TUNEL assay. Detection of 1-4 TUNEL+ cells in the stroma of the naïve, no-decorin-delivered (FIG. 12A), and decorin-delivered (FIG. 12B) rabbit corneas suggests that the AAV5 vector is noncytoxic to keratocytes. The 6-10 TUNEL+ cells observed in the corneal epithelium were due to its replenishment. The presence of a similar number of DAPI-stained nuclei in no-decorin-delivered (FIG. 12A) and decorin-delivered (FIG. 12B) rabbit corneas also suggests that optimized AAV-based decorin delivery into stroma does not alter keratocyte populations. These results suggest that the tested gene transfer modality is safe for in vivo corneal gene therapy.

Testing also addresses the concern that decorin is known to bind to collagen and regulate fibrillogenesis. Results showed that AAV-mediate decorin gene therapy does not jeopardize corneal collagens. Specifically, it was examined whether decorin gene therapy affects collagen fibril diameter and/or arrangement in the cornea, since collagen fibril diameter and arrangement in the cornea play an important role in corneal transparency.

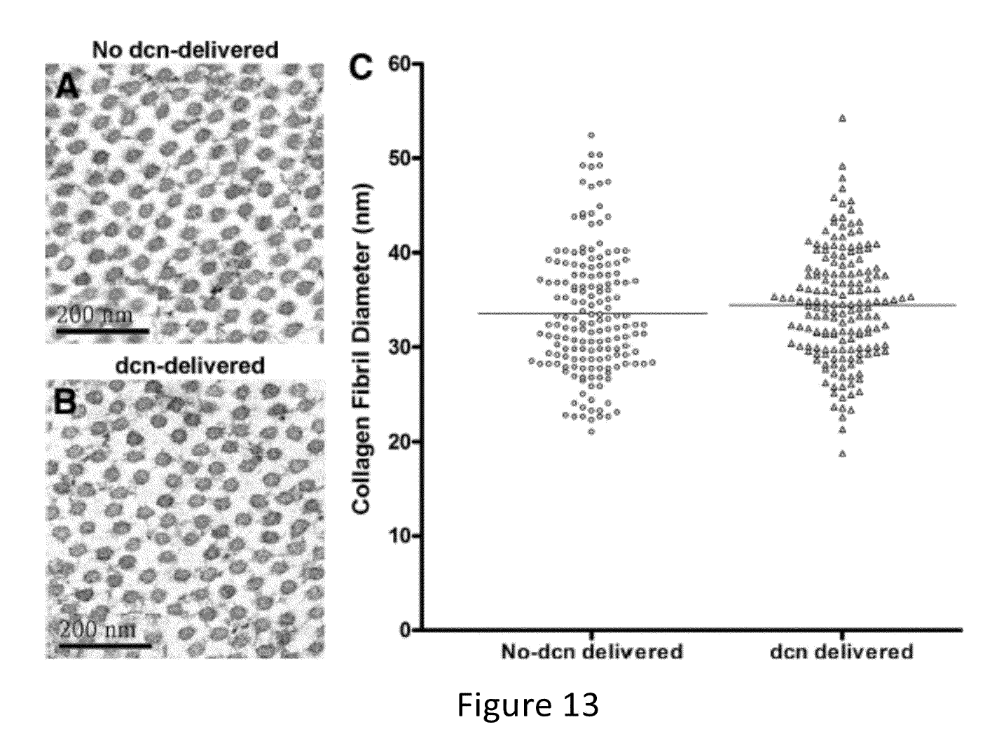

FIGS. 13A to 13C show the effect of decorin gene therapy on collagen fibril diameter determined by transmission electron microscopy of no-decorin-delivered control. FIG. 13A shows the diameter and arrangement of collagen fibrils in no-decorin-delivered rabbit cornea, and FIG. 13B shows the diameter and arrangement of collagen fibrils in decorin-delivered rabbit cornea. No significant difference between the collagen fibrils arrangement or diameter were detected in decorin-delivered and un-delivered rabbit corneas suggesting that AAV5-delivered decorin in the stroma does not jeopardize geometric arrangement of collagen fibrils in the rabbit cornea. The quantification of collagen fibril diameter data shown in FIG. 13C did not detect any significant differences between the corneas of the two groups.

Optical coherence tomography biomicroscopy performed in rabbit eyes provided additional support to the conclusion as no significant change in corneal thickness up to 4 weeks was observed among the naïve, no-decorin-delivered, and decorin-delivered rabbit corneas. Moreover, the visual and slitlamp clinical eye examinations did not detect inflammation, redness, opacity, mucous, or other discharges in the rabbit eye, and optical coherence tomography imagining did not find any distortion in rabbit corneal thickness or curvature.

While the invention has been described in connection with specific embodiments thereof, it will be understood that the inventive device is capable of further modifications. This patent application is intended to cover any variations, uses, or adaptations of the invention following, in general, the principles of the invention and including such departures from the present disclosure as come within known or customary practice within the art to which the invention pertains and as may be applied to the essential features herein before set forth.

### Example 3

### Material and Methods

Animals:

The Institutional Animal Care and Use Committee of the University of Missouri-Columbia, Mo. USA USA (ID#4279 and 6487) and Harry S. Truman Memorial Veterans' Hospital Columbia, Mo. USA (ID#0041 and 0089) approved the study. Animals were treated in adherence to the principles of the ARVO Statement for the Use of Animals in Ophthalmic and Vision Research. New Zealand White rabbits (Myrtle laboratories Inc., Thompson's Station, Tenn.) weighing 2.5-3.0 kg were used in this study. Rabbits were anesthetized by intramuscular injection of ketamine hydrochloride (50 mg/kg) and xylazine hydrochloride (10 mg/kg) for performing PRK, VEGF-implantation, stereo- and slit-lamp biomicroscopy.

AAV5 Vector Generation

The AAV5 expressing green fluorescent protein gene (AAV5-GFP) titer produced at the Gene Therapy Vector Core Lab, University of Florida, Gainesville, Fla. was procured from Prof. Gregory S. Schultz and Dr. Vince A. Chido. Following an earlier reported method the AAV2 plasmid pTRUF11 expressing fluorescent green protein gene under control of a hybrid promoter (cytomegalovirus enhancer and chicken b-actin) and simian virus 40 polyadenylation site was packaged into AAV5 (Zolotukhin S, Potter M, Zolotukhin I, Sakai Y, Loiler S, et al. (2002) Production and purification of serotype 1, 2, and 5 recombinant adeno-associated viral vectors. Methods 28: 158-67). In brief, AAV5 vector was produced by the 2-plasmid, co-transfection method. One Cell Stack (Corning Inc., Corning, N.Y., USA) with approximately 16109 HEK 293 cells was cultured in Dulbecco's Modified Eagle's Medium (Hyclone Laboratories, Inc. Logan Utah, USA), supplemented with 5% fetal bovine serum and antibiotics. A CaPO4 transfection precipitation was set up by mixing a 1:1 molar ratio of AAV2 plasmid DNA containing GFP and AAV5 rep-cap helper plasmid DNA. This precipitate was applied to the cell monolayer and the transfection was allowed to incubate at 37° C., 7% CO₂ for 60 h. The cells were then harvested and lysed by freeze/thaw cycles and subjected to discontinuous iodixanol gradients centrifugation at 350,000 g for 1 h. This iodixanol fraction was further purified and concentrated by column chromatography on a 5-ml HiTrap Q Sepharose column using a Pharmacia AKTA FPLC system (Amersham Biosciences, Piscataway, N.J., USA). The vector was eluted from the column using 215 mM NaCl buffer, pH 8.0, and the rAAV peak collected. AAV5 GFP vector-containing fraction was then concentrated and buffer exchanged in Alcon BSS with 0.014% Tween 20, using a Biomax 100 K concentrator (Millipore, Billerica, Mass., USA). Vector was titered for DNAse-resistant vector genomes by Real-Time PCR relative to a standard.

AAV5 Transduction to Rabbit Cornea

Twenty-eight rabbits ware used for the study. Only one eye of each rabbit selected randomly was used for the experiment. Sixteen rabbits were divided into two groups for the optimization of gene delivery parameters for the cornea. Rabbits of AAV5-treated group (n=10) received 100 ml titer (6.561012 vg/ml) of AAV5 expressing green fluorescent protein gene under control of cytomegalovirus enhancer and chicken b-actin promoters topically for 2 minutes on de-epithelialized cornea via a custom hairdryer based vector delivery technique reported recently (Mohan R R, Sharma A, Cebulko T C, Tandon A (2010) Vector delivery technique affects gene transfer in the cornea in vivo. Mol V is 16: 2494-2501). The control group (n=6) received balance salt solution (BSS) topically using similar conditions. Twelve rabbits were used to evaluate the efficiency of optimized gene transfer parameters for delivering genes into diseased corneas namely rabbit corneal scarring model (n=6) and rabbit neovascularization model (n=6) were used. The AAV5 vector was topically applied to scarred rabbit cornea 4 weeks after PRK (n=6) or neovascularized rabbit corneas 5-day after VEGF implantation (n=6) using similar vector volume, titer, delivery technique, and experimental conditions. The contralateral eyes served as a naive control.

Corneal Neovascularization and Haze Generation

Neovascularization in rabbit cornea was induced by corneal micro-pocket assay [44]. Rabbits were anesthetized with ketamine and xylazine, and 3-4 drops of 0.5% topical proparacaine hydrochloride solution (Alcon, Ft. Worth, Tex., USA) was applied to the eye prior to cornea micropocket surgery. Only one eye of each animal was used for surgical procedure. The contralateral eye served as naive control. A wire speculum was positioned in the eye and a sucralfate-hydron pellet containing 650 ng of VEGF (PeproTech, Rocky Hill, N.J.) was implanted into the cornea after making a micropocket in the cornea using standard surgical tools. Triple antibiotic ointment (Alcon) was applied to the surface of the cornea after pellet implantation to prevent infection. The ingrowth of blood vessels in the cornea towards the VEGF implant started from day 2, peaked around day 10 and continued to grow progressively up to 15 days before regressing.

Haze in rabbit cornea was produced by performing photorefractive keratectomy (PRK) surgery in an anaesthetized rabbit (Sharma A, Mehan M M, Sinha S, Cowden J W, Mohan R R (2009) Trichostatin A inhibits corneal haze in vitro and in vivo. Invest Ophthalmol V is Sci 50: 2695-2701). Topical proparacaine hydrochloride 0.5% (Alcon, Ft. Worth, Tex., USA) was applied to each eye just before PRK. A wire lid speculum was positioned and a 7 mm-diameter area of epithelium overlying the pupil was removed by scraping with a #64 blade (Beaver; Becton-Dickinson, Franklin Lake, N.J., USA). The 29.0 diopter PRK surgery with a 6 mm ablation zone on the central stroma was performed using the Summit Apex excimer laser (Alcon, Ft. Worth, Tex.). Only one eye from each animal was used for PRK and the contralateral eye served as naive control. The corneal haze in animals peaked 4 weeks after PRK.

Clinical and Slit-Lamp Biomicroscopy

The health of the cornea in eyes of live rabbits was examined by visual clinical and slit-lamp microscopic (BX 900 Slit Lamp, Haag-Streit-USA, Mason Ohio) examinations before and after AAV5 application in normal and diseased (hazy or neovascularized) rabbit corneas by two ophthalmologists and a researcher, independently and in a blinded manner while animals were under general anesthesia. Thereafter, corneal health was monitored every third day with a hand-held slit-lamp microscope (SL-15, Kowa Optimed Inc., Torrance, Calif.). Photographs of the cornea were taken with a digital camera attached to the BX 900 slit-lamp microscope.

Tissue Collection

Rabbits were humanely euthanized with pentobarbitone (150 mg/kg) overdose under general anesthesia at selected time points. Rabbit corneas were removed with forceps and sharp Westcott scissors and cut into 2 equal halves. One half was embedded in liquid optimal cutting temperature (OCT) compound (Sakura FineTek, Torrance, Calif.) within a 24 mm624 mm65 mm mold (Fisher, Pittsburgh, Pa.) and snap frozen. Frozen tissue blocks were maintained at 280uC. Tissue sections were cut 7 mm thick with a cryostat (HM 525 M, Microm GmbH, Walldorf, Germany). Sections were placed on 25 mm675 mm61 mm microscope Superfrost Plus slides (Fisher), and maintained frozen at 280uC until staining. The other half of rabbit corneal tissues was snap frozen directly in liquid nitrogen for isolating RNA, DNA or protein.

Immunohistochemistry and Hematoxylin and Eosin Staining

Corneal tissues were stained with hematoxylin and eosin (H & E). Immunofluorescence staining for alpha smooth muscle actin (αSMA), a marker for myofibroblasts, was performed using mouse monoclonal primary αSMA antibody (1:200 dilution, catalog no. M0851, Dako, Carpinteria, Calif.). Tissue sections were incubated with 2% bovine serum albumin for 30 minutes at room temperature and then with αSMA monoclonal antibody for 90 minutes. For the detection of the primary antibody, Alexa 488 goat anti-mouse IgG secondary antibody (1:1000 dilution; catalog no. A11001, Invitrogen Inc., Carlsbad, Calif.) for 1 hour was used.

Blood vessel formation was confirmed with tomato lectin staining which entailed the incubation of corneal sections with 20 mg/ml Texas red-conjugated tomato lectin (cat #TL-1176; Vector laboratories, Burlingame, Calif.) for 90 min. Tissue sections were washed in HEPES buffer and mounted using Vectashield medium containing 49-6-diamidino-2-phenylindole (DAPI; Vector laboratories). The stained sections were viewed and photographed with a Leica fluorescent microscope (Leica DM 4000B; Leica) equipped with a digital camera (SpotCam RT KE).

Immunoblotting

Protein lysates were prepared by homogenizing corneas in protein lysis buffer containing protease inhibitor cocktail (Roche Applied Sciences, Indianapolis, Ind.). Total protein was determined with Bradford assay. The same amount of protein of each sample was suspended in Laemmli denaturing sample buffer, vortexed and heated for 10 min at 70uC. The proteins were resolved on 4-20% SDS-PAGE gel and transferred onto 0.45 mm pore size PVDF membrane (Invitrogen, San Diego, Calif.). The membrane was incubated with GFP (cat #sc-33856; Santa Cruz) or β-actin (cat #sc-69879; Santa Cruz) primary antibody followed by alkaline phosphatase-conjugated anti-goat or anti-mouse secondary antibody (Santa Cruz). The bands were visualized by NBT/BCIP.

Stereo-Biomicroscopy and Confocal Microscopy

Fluorescent stereomicroscope (model MZ16F, Leica) was used to track GFP expression in the eye of live rabbits under general anesthesia. The spatial localization of delivered-GFP gene in whole-mounts of normal cornea and thick tissue sections of damaged corneas was determined with confocal microscope (TCSSP; Leica or Radiance 2000; Bio-Rad) using corresponding lasers for DAPI and GFP. The paraformaldehyde (4%) fixed corneal whole-mount tissues were stained with DAPI for 3 days to stain nuclei. The 20 mm thick corneal sections of the damaged rabbit corneas were subjected to triple staining (nuclei with DAPI, cells expressing-GFP, and cells expressing SMA or lectin). The Z-stacks were generated in 0.45 mm increments and 3-D reconstructions were created by computer using Velocity software (Impro Vision Inc., Lexington, Mass.). The 3-D images were rotated 360u for spatial and perceptual visualization of the corneal regions. The exact location and quantity of the EGFP positive cells in the cornea were measured with Velocity software (Impro Vision) and NIH Image J software.

Slot Blot Analysis

The copies of delivered plasmid were determined with slot blot analysis. Frozen corneal tissues were ground in liquid nitrogen and DNA was isolated using the DNA easy kit (Qiagen, cat #69504). The standards were prepared using 104-1011 copies of decorin gene cloned into pTRUF11 vector. The DNA probe was prepared by digesting 5 mg of decorin plasmid with Not1 restriction enzyme and labeling 1 mg of isolated decorin fragment with digoxigenin (DIG)-labeled UTP, using DIG starter Kit II (catalog no. 11585614910 Roche Applied Science, Indianapolis, Ind.). Two microliters of the standard as well as the DNA isolated from corneal tissues was denatured by alkali and heat treatment. Denatured DNA samples were blotted onto nylon membrane using slot blot apparatus (BioRad lab) and were UV-cross linked. The membrane was hybridized with 300 ng of digoxigenin (DIG)-labeled probe overnight at 30uC, followed by incubation in 1:5000 anti-digoxigenin-AP antibody. Chemiluminescent detection was used following vendor's instructions (catalog no. 11585614910 Roche Applied Science, Indianapolis, Ind.) and membrane was exposed to X-ray film. Image J 1.386 image analysis software was used to determine delivered gene copies in samples by measuring dot intensities of samples and comparing the data with standards.

### Results

Characterization of AAV5-Mediated Gene Transfer in Rabbit Cornea

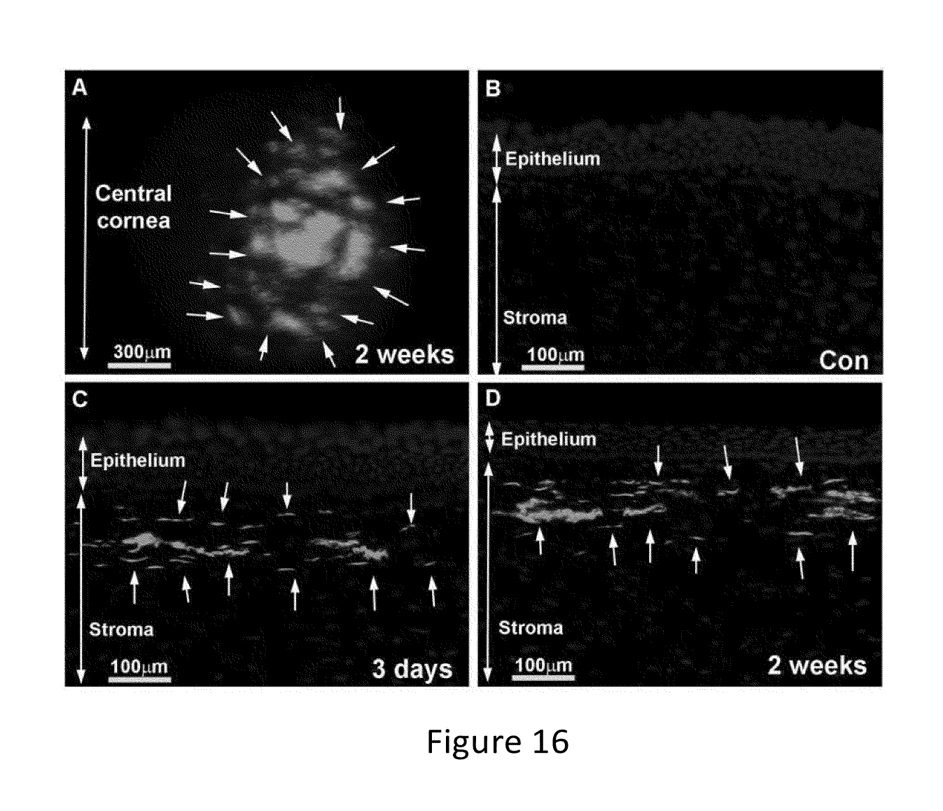

FIG. 16 shows AAV5-mediated delivery of GFP gene in the normal rabbit cornea detected using stereo- (FIG. 16A) and fluorescent- (FIGS. 16B-16D) microscopy. Rabbits were subjected to fluorescence imaging every 12 h for the first 3 days after AAV5 vector application, and thereafter once a week until euthanasia. All rabbit corneas showed initial appearance of GFP gene expression at day-3 (FIG. 16C), which reached its maximum level at day-7. FIG. 16D shows a representative image of peak level of GFP expression in rabbit corneal tissue section detected at 2-week time point. No fluorescence was detected in corneas of early tested time points (12 h, 24 h, 48 h or 60 h). Rabbit corneas of later time points (2-week, 4-week and 16-week) showed fluorescence levels similar to the levels of 7-day time point. These observations suggest that AAV5 delivered transgene expression first appeared between 60 h to 72 h after vector application, continued to increase for the next 4 days, peaked at day-7, and maintained high transgene expression up to the longest tested time point of 16-week (4 months) in the rabbit corneas in vivo.

Quantification of AAV5-Mediated Gene Transfer

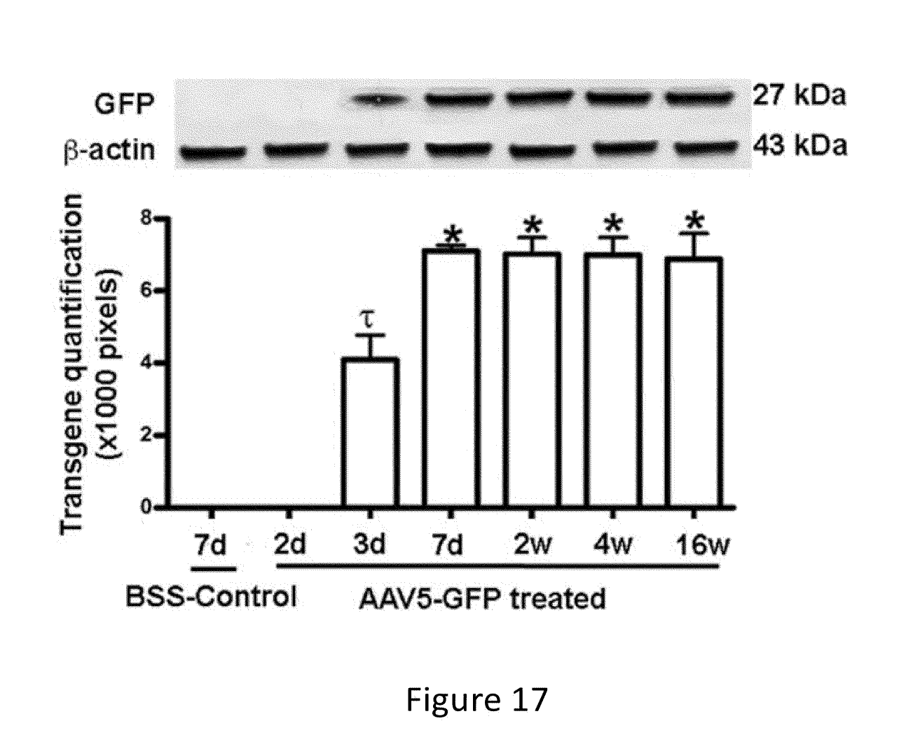

The level of AAV5 delivered GFP gene expression was quantified using western blot. FIG. 17 shows the levels of delivered GFP protein in rabbit corneas at various tested time points (2-day, 3-day, 7-day, 2-week, 4-week and 16-week) after single topical application of AAV5. The digital quantification of the western blot depicting the average pixels of three independent experiments is shown in FIG. 17. The first detectable expression was noted at day-3 (4099 pixels6682). The maximum GFP expression was observed at day-7 (7100 pixels6154), which was significantly higher compared to day-3 (p, 0.05) and balanced salt solution (BSS)-treated controls (p, 0.01). Also, the GFP expression detected at other tested time points of 2-weeks (7021 pixels6462), 4-weeks (6998 pixels6473) and 16 weeks (6880 pixels6698) was significantly (p, 0.05) higher than the GFP expression detected at day-3 as well as BSS-treated controls. No GFP expression was detected in BSS-treated control rabbit corneas. Equal loading of protein was confirmed by the detection of similar intensity b-actin bands.

Spatial Localization of AAV5-Mediated GFP Gene Transfer

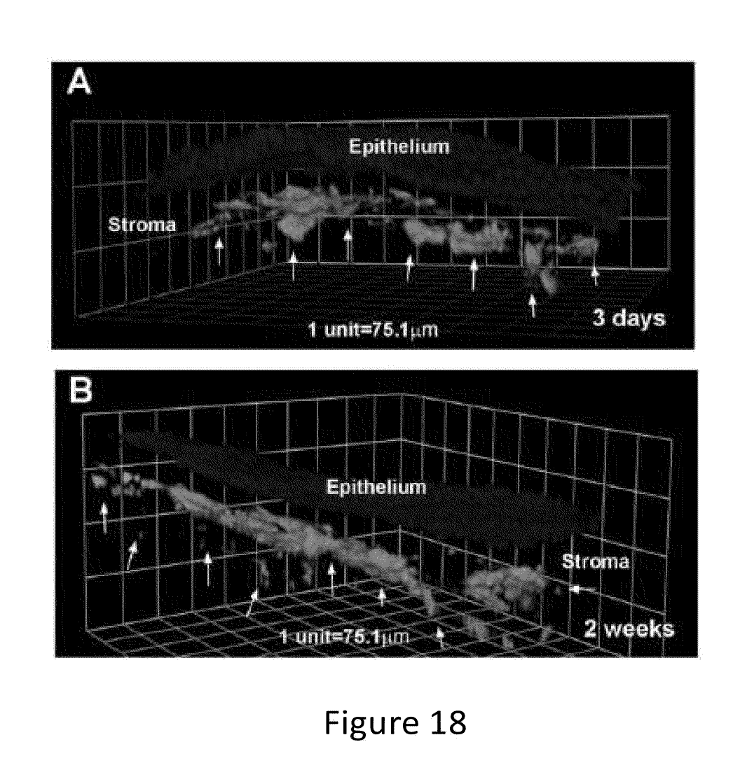

To detect spatial localization of AAV5-mediated gene transfer in the rabbit cornea, we performed confocal microscopy in rabbit corneal tissues collected 3 days and 2 weeks after AAV5 application. The three-dimensional z-stack confocal images presented in FIG. 18 reveal localization of GFP in rabbit corneas of 3-day (A) and 2-week (B) time points. As evident from this figure, the delivered-GFP gene expression was detected in the anterior stroma just below the corneal epithelium. No transgene expression was detected either in the corneal epithelium or posterior stroma or corneal endothelium. These observations suggest that AAV5 vector administered to the rabbit cornea with defined vector-delivery technique provided tissue-selective localized gene delivery in the anterior stroma.

Determination of Delivered-GFP Gene Copies with AAV5

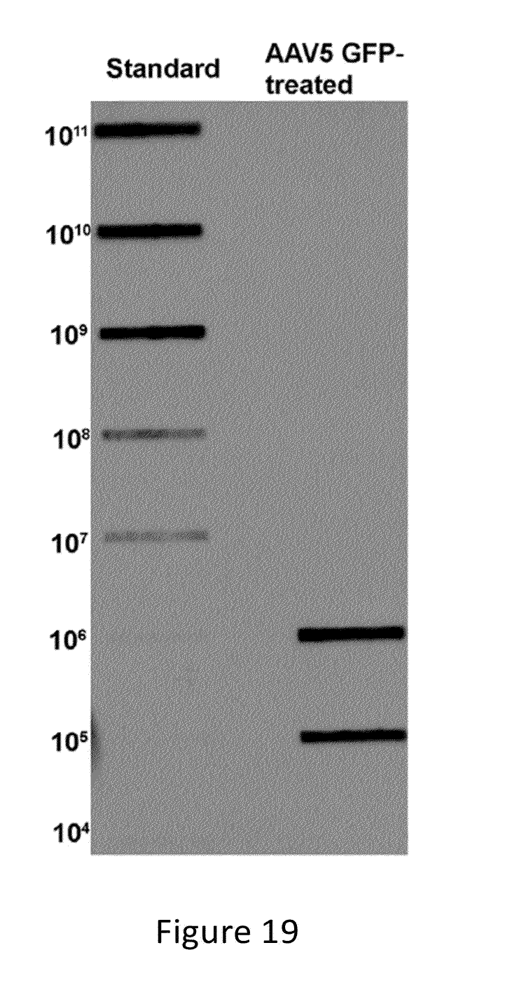

To understand the correlation between delivered-GFP gene copy number and expression of delivered-GFP protein, we measured AAV5-delivered GFP gene copy number in rabbit corneas using slot blot. FIG. 19 shows the gene copy number delivered in two separate rabbit corneas detected at 2-week time point using slot blot method. Densitometric analysis revealed that 108-1010 genomic copies of transgene were detected in the rabbit corneas. This data complement the results of immunocytochemistry (FIG. 16) and western blotting (FIG. 17).

AAV5-Mediated Gene Delivery in Diseased Rabbit Corneas

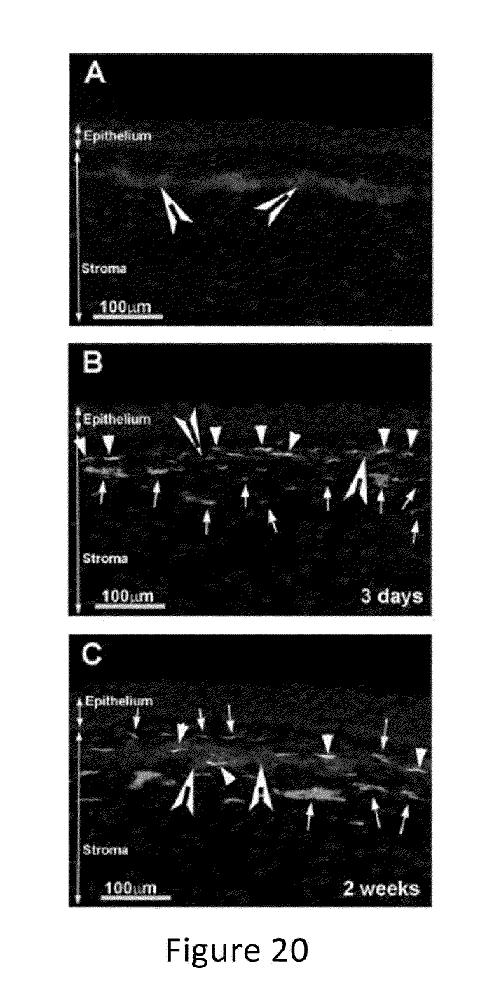

Diseases affecting the corneas are associated with significant alterations in corneal homeostatic and/or cellular phenotype. Thus, we raised a question “do gene transfer parameters optimized using normal rabbit corneas are applicable for the diseased cornea?” To answer this question we used two most acceptable in vivo rabbit disease models; the PRK-based corneal scarring model and the VEGF-induced corneal neovascularization model to test the potential of optimized tissue-targeted gene transfer approaches using AAV5 for treating corneal diseases such as corneal fibrosis and corneal neovascularization. The gene transfer data observed in scarred rabbit cornea is shown in FIG. 20. The detection of transdifferentiated keratocytes (myofibroblasts) with αSMA (a fibrosis biomarker) immunostaining (FIG. 20A) confirmed the scarring in rabbit corneas induced by PRK surgery. AAV5-delivered GFP gene expression detected at 3-day (FIG. 20B) and 2-week (FIG. 20C) time points. As evident from FIGS. 20B and 20C, AAV5 delivered significant levels of GFP in the anterior stroma of the scarred rabbit cornea. Furthermore, co-localization of GFP and αSMA suggests that transgene was also delivered into transdifferentiated keratocytes (myofibroblasts) in addition to keratocytes by this technique. These observations revealed that defined gene transfer parameters are efficient for corneal gene therapy.

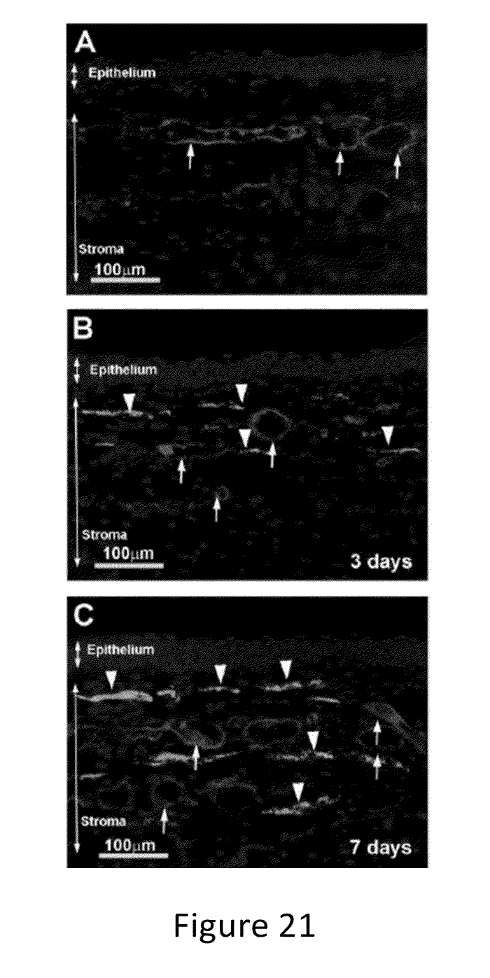

Next, we evaluated the efficiency of defined gene transfer parameters using AAV5 for delivering genes into neovascularized rabbit corneas. FIG. 21 shows that a single 2 minutes topical application of AAV5-GFP vector on the rabbit stroma delivered significant levels of transgene into neovascularized rabbit corneas further validated suitability of optimized parameters and tested AAV5 vector for delivering therapeutic genes in diseased corneas. The increased GFP expression detected at 7-day (FIG. 21C) compared to 3-day (FIG. 21B) time point suggests that neovascularization did not alter kinetics of AAV5-mediated gene transfer. GFP delivery and blood vessel formation in the rabbit corneal section in FIG. 21 is shown with GFP immunostaining and lectin staining.

Safety Determinations with Slitlamp Biomicroscopy and Histology of AAV5-Treated Rabbit Corneas

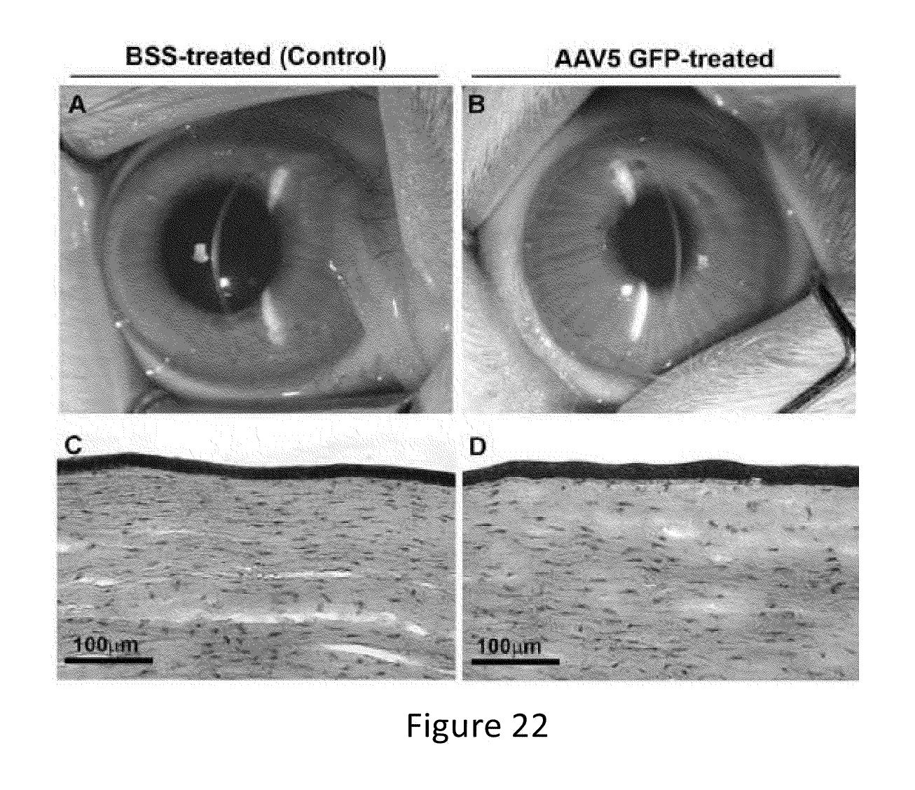

To analyze the effects of AAV5 on corneal health, visual and slit-lamp clinical examinations were performed in the eyes of live rabbits 1-day, 2-day, 3-day, 7-day and 4-week after BSS or AAV5 application. Neither BSS (FIG. 22A) nor AAV5-GFP (FIG. 22B) treated rabbit eyes showed inflammation, unusual discharge, swelling, redness or infection in the eye during clinical examination suggesting that AAV5 vector and used topical delivery technique are safe for the rabbit cornea. The hematoxylin and eosin-staining of BSS-treated control (FIG. 22C) and AAV5-treated rabbit corneas (FIG. 22D) collected at various time points did not exhibit any apparent structural abnormalities or abnormal infiltration of inflammatory cells in the rabbit cornea further confirming the safety of AAV5 vector for corneal gene delivery.

### Example 4

### Material and Methods

Reagents

The cell culture reagents, pcDNA3.1/V5-HisA,B,C mammalian gene expression vector, transfection reagents, and secondary antibodies were purchased from Invitrogen, San Diego, Calif., USA. The RNA extraction kit was purchased from Qiagen Inc., Valencia, Calif. and ImProm-II Reverse Transcription kit to prepare cDNA was obtained from Promega, Madison, Wis. The iQ SYBR green super mix and JumpStart PCR mix were obtained from Bio-Rad Laboratories, Hercules, Calif. and Sigma-Aldrich, St Louis, Mo., USA, respectively. TGFβ1 was purchased from PeproTech Inc, Rocky Hill, N.J., USA. The decorin and SMA primary antibodies were either purchased from Santa Cruz Biotechnology, Santa Cruz, Calif. or R&D System Minneapolis, Minn. The decorin antibody was also procured from Dr. Larry W Fisher, National Institute of Dental and Craniofacial Research, NIH, Bethesda, Md., USA. The DAPI containing mounting medium was purchased Vector Laboratories, Inc., Burlingame, Calif., USA.

Human Corneal Fibroblasts Culture

Primary HSF cultures were generated from donor human corneas obtained from eye banks using method described previously [Sharma et al., 2009]. Briefly, The epithelium and endothelium of the cornea were removed by gentle scraping with scalpel blade after washing the tissue with Minimum Essential Medium (MEM). The cornea was cut into small pieces, placed on culture dishes, and incubated in humidified CO₂ (5%) incubator at 37° C. in MEM supplemented with 10% fetal bovine serum for 3-5 weeks to obtain human corneal fibroblasts. Seventy percent confluent HSF cultures (1-3 passage) were used for experiments. Myofibroblasts were produced by culturing HSF cultures under serum free conditions in presence of TGFβ1 (1 ng/ml). TGFβ1 was purchased from PeproTech Inc, Rocky Hill, N.J. and cell culture reagents were purchased from Invitrogen, San Diego, Calif., USA or Sigma-Aldrich, St Louis, Mo., USA.

Vector Generation, Transfection and Selection of Stable Clones

The pcDNA3.1/V5-HisB mammalian gene expression vector system (Invitrogen, San Diego, Calif.) was used. A PCR-amplified human decorin (Accession #NM_{—}001920) cDNA (−1.1 Kb) was cloned into pcDNA3.1/V5-His A vector employing standard molecular biological techniques. Restriction mapping and DNA sequencing were used to confirm the nucleotide sequence of the pcDNA3.1/V5-HisB-decorin (hereafter denoted as pcDNA3.1-decorin) vector construct. The transfection of pcDNA3.1-decorin plasmid into HSF was performed with Lipofectamine 2000 (Invitrogen) following vendor's instructions. Briefly, for each transfection, 10 μg plasmid DNA in 100 μl of MEM was added to 100 μl Lipofectamine™ 2000 diluted with MEM, and incubated at room temperature for 20 minutes or until solution became cloudy. The DNAL-ipofectamine solution was added drop wise to the cells containing 1.5 ml medium by rocking the plate back and forth. Cultures were incubated at 37° C. in a CO2 (5%) incubator for 6-8 hours, washed with medium, and incubated 48-72 hrs for transient transgene expression. The stably transfected clones were identified by growing cultures in presence of MEM medium supplemented with 10% serum and geneticin (250 μg/ml).

Cellular Morphology and Viability

The cellular morphology of clones was monitored with Leica DMIL phase-contrast microscope equipped with Leica DFC290 imaging system. Cultures were visualized at different time intervals and their phenotype was recorded using digital camera. Trypan blue assay was performed as reported earlier following manufacturer's instructions (BRB 2010). Briefly, at selected time points, cells were trypsinised and mixed with equal amounts of 0.4% trypan blue solution (Invitrogen). Dead cells with ruptured membranes and live cells were counted with Neubauer's chamber. Cellular viability was calculated as a percent.

RNA Extraction, cDNA Synthesis and Quantitative Real Time PCR

Total RNA from the cells was extracted using RNeasy kit (Qiagen Inc., Valencia, Calif., USA) and was reverse-transcribed to cDNA following vendor's instructions (Promega, Madison, Wis., USA). Real-time PCR was performed using iQ5 real-time PCR Detection System (Bio-Rad Laboratories, Hercules, Calif., USA) and hot start PCR were performed using JumpStart Taq DNA polymerase (Sigma, St Louis, Mo., USA). A fifty microliters real-time PCR reaction mixture containing 2 μl cDNA (250 ng), 2 μl forward (200 nM) 2 μl reverse primer (200 nM) and 25 μl 2×iQ SYBR green super mix (Bio-Rad Laboratories) was run at universal cycle (95° C. for 3 min, 40 cycles of 95° C. 30 sec followed by 60° C. 60 sec) following vendor's instructions [Sharma et al 2009]. A fifty microliters conventional hot start polymerase chain reaction containing cDNA (250 ng), forward primers (36 pg/ml), reverse primers (36 pg/ml), dNTP (400 mM of each), and JumpStart Taq polymerase (4 units) in a 10 mM Trizma-HCl, pH 8.3, 50 mM KCl, 1.5 mM MgCl2, and 0.001% gelatin was run using cycle conditions (95° C. for 4 min, followed by 40 cycles of 95° C. for 1 min, 55° C. for 30 sec, and 72° C. for 1 min, with a final cycle at 72° C. for 10 min) reported earlier [Mohan et al., 2003]. The forward and reverse primers used in this study are given in Table-1. Beta actin was used to test the quality of cDNA and as a house keeping gene in real-time PCR. The threshold cycle (Ct) was used to detect the increase in the signal associated with an exponential growth of PCR product during the log-linear phase. The relative expression was calculated using the following formula, 2-ΔΔCt. The ΔCt validation experiments showed similar amplification efficiency for all templates used (difference between linear slopes for all templates less than 0.1). Three independent experiments were performed and the average (±SEM) results are presented in graphic form.

Protein Extraction and Immunoblotting

Cells were washed with ice-cold PBS, lysed with 0.6 ml RIPA buffer (50 mM Tris-HCl, pH 7.5, 150 mM NaCl, 1% NP40, 0.5% sodium deoxycholate) containing protease inhibitor cocktail (Roche Applied Sciences, Indianapolis, Ind., USA) and transferred to 1.5 ml microfuge tubes. DNA was sheared by passing cell lysates through a 21-gauge needle at least ten times and debris was removed by centrifugation at 14 000 g at 4° C. The protein concentration of the lysates was determined using the Bio-Rad assay (Sharma A, Mehan M M, Sinha S, Cowden J W, Mohan R R. Trichostatin-A inhibits corneal haze in vitro and in vivo. Invest Ophthalmol V is Sci 2009; 50:2695-2701. [PubMed: 19168895]).

The western blotting was performed by denaturing protein samples in Laemmli's sample buffer containing β-mercaptoethanol at 70° C. for 10 min. Proteins were resolved on 4-10% SDS-PAGE, and electrophoretically transferred to a 0.45-μm pore size PVDF membrane using Xcell-II blot module (Invitrogen). The membrane was blocked with 5% nonfat dry milk in TBST for 1 hr and probed with SMA, decorin or

GAPDH primary antibodies (1:100 dilution) followed by secondary anti-mouse- or goat antibodies (1:2000 dilution). The bands were visualized by NBT/BCIP.

Immunocytochemistry

Immunofluorescent staining for SMA, a myofibroblast marker responsible for corneal fibrosis, was performed using mouse monoclonal antibody for SMA. Myofibroblast formation in decorin-transfected, naked vector-transfected and un-transfected HSF was stimulated by culturing clones in the presence of TGFβ1 (1 ng/ml) under serum-free conditions. At study endpoint, cultures were washed twice with PBS and incubated at room temperature with a mouse monoclonal antibody for SMA (DAKO) at a 1:200 dilution in 1×PBS for 90 minutes and with secondary antibody Alexa 488 or 594 goat anti-mouse IgG (Invitrogen) at a dilution of 1:500 for 1 hour. Cells were mounted with Vectashield containing DAPI (Vector Laboratories) to allow visualization of nuclei. Irrelevant isotype-matched primary antibody, secondary antibody alone, and tissue sections from naïve eyes were used as negative controls. Culture slides were visualized under Leica fluorescent microscope (Leica, Wetzlar, Germany) and photographed with a digital camera (SpotCam RT KE, Diagnostic Instruments Inc., Sterling Heights, Mich., USA) equipped to microscope. The SMA-stained cells in ten randomly selected areas were counted per 200× and/or 400× magnification field.

Statistical Analysis

Statistical analysis was performed using two-way analysis of variance (ANOVA) followed by Bonferroni multiple comparisons test for cell toxicity assay. The results were expressed as mean±standard error of the mean (SEM). The real-time PCR data was analyzed using one way ANOVA followed by Tukey's multiple comparison tests. A “p value”<0.05 was considered significant. The immunoblotting data was analyzed using image J 1.38× image analysis software (NIH, USA).

### Results

Detection of Decorin Transcript in HSF Clones

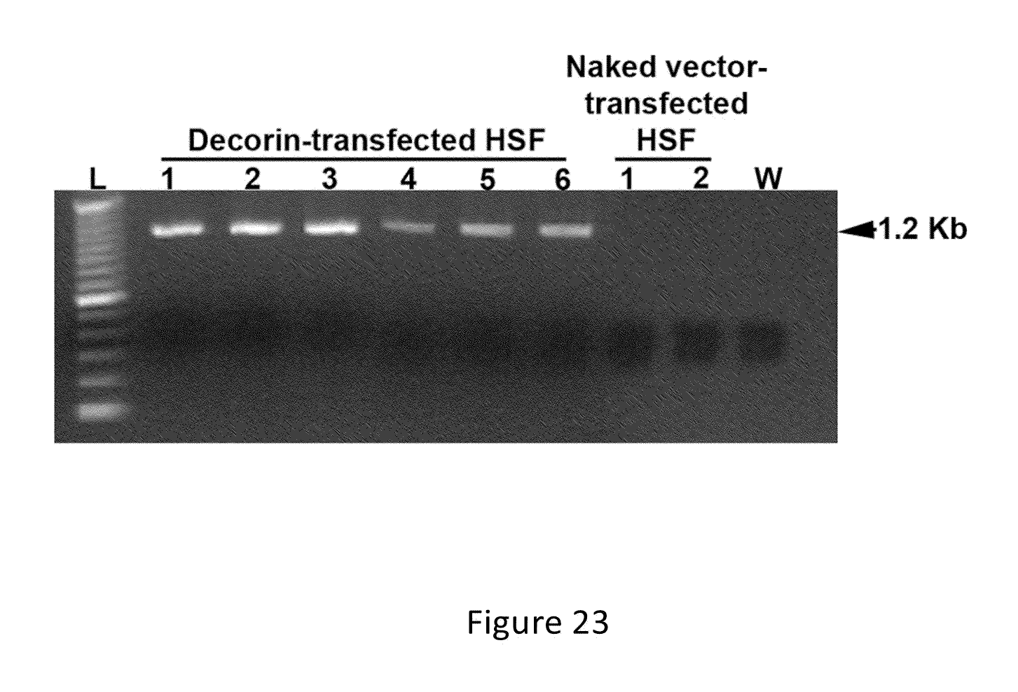

Various combinations of forward and reverse primers designed from decorin and vector DNA sequences were used in PCR to amplify decorin cDNA originating from RNA in HSF transfected with pcDNA3.1-decorin or control vector. FIG. 23 shows the amplification bands detected in tested clones. Detection of DNA fragment of about 1.2 Kb corresponding to expected amplification product size confirmed new decorin mRNA transcription from the characterized decorin over-expressing clones. Control cultures showed no decorin amplification indicating that decorin expression in.

Immunodetection of Decorin Transfected HSF Clones

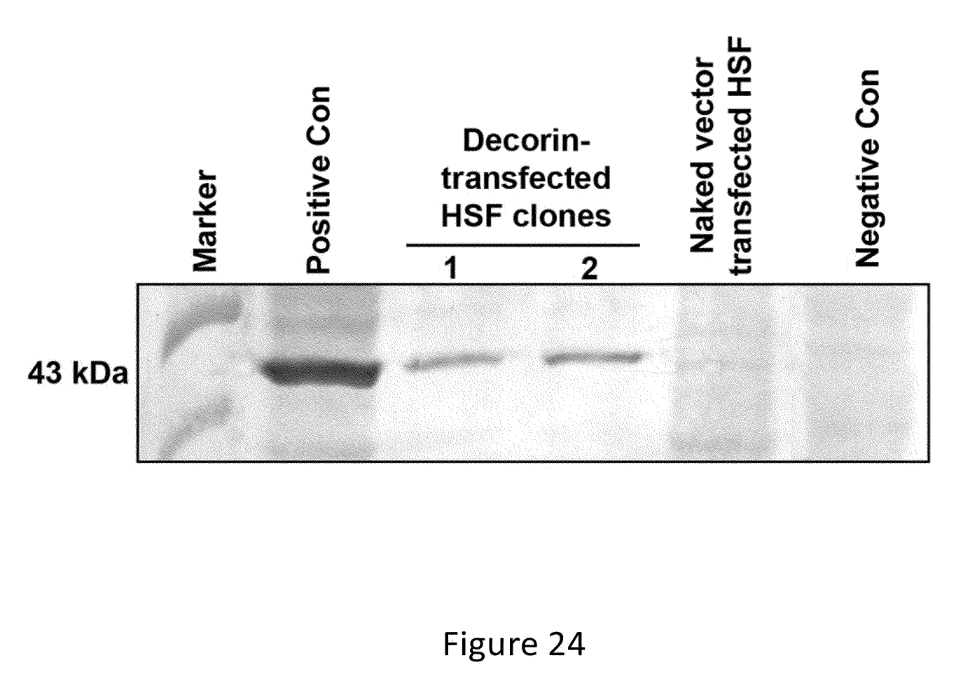

The protein cell lysates prepared from HSF clones transfected with pcDNA3.1-decorin or control vector were analyzed by western blotting using anti-decorin antibodies. FIG. 24 shows representative immunoblotting data. The HSF clones transfected with pcDNA3.1-decorin revealed a positive signal for decorin while no decorin signal was detected in clones transfected with naked vector.

Cellular Morphology and Viability

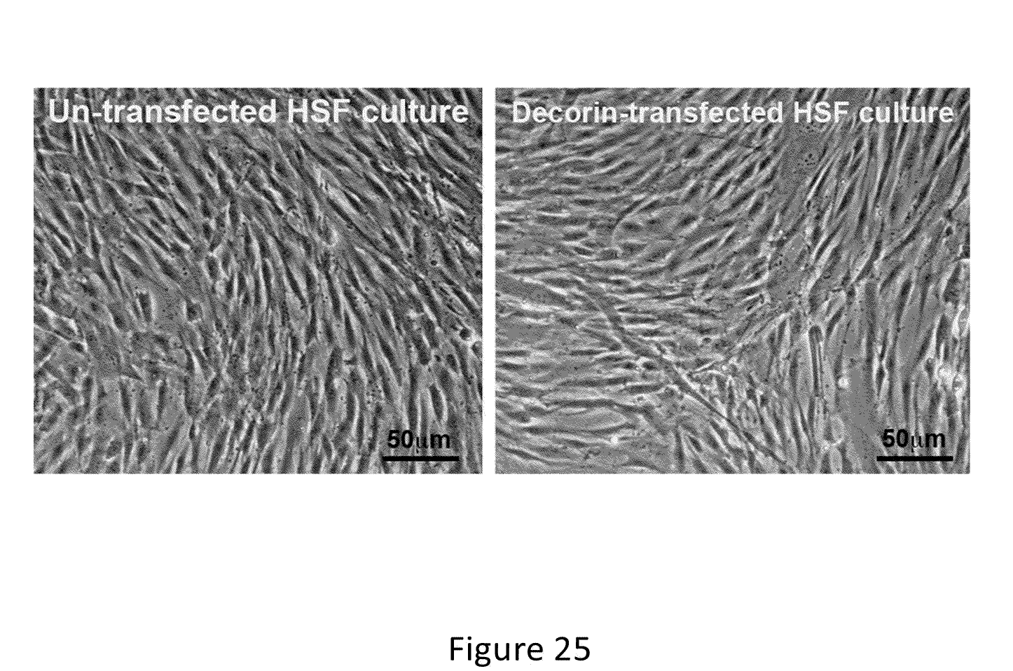

The cellular morphology of HSF clones transfected with pcDNA3.1-decorin or control vector were examined under phase-contrast microscopy. Both, decorin- and naked-vector transfected HSF, clones presented phenotype exhibited by normal human corneal fibroblasts. The characterized clones were long, spindle-shaped and possessed typical fibroblastic morphology when grown to confluence in medium containing 10% serum. FIG. 25 shows cellular morphology of HSF clones transfected with pcDNA3.1-decorin or naked vector in confluent cultures. This data suggest that decorin gene transfer into HSF do not alter cellular morphology.

The effect of decorin gene transfer on HSF viability was determined by trypan assay. Tested decorin- and naked-vector transfected clones showed varied cellular viability. Both, the decorin- and naked-vector transfected HSF clones at early passages (up to 3) showed >93% viable cells very similar to normal untransfected HSF. A gradual decrease in cellular viability (10% or more) was observed in decorin- and naked-vector transfected tested clones in later passages. The clones were not sub-cultured after passage 6. This data suggest that decorin over-expression in HSF does not affect its viability.

Effect of Decorin Transfection on Fibrosis-Related Genes in HSF

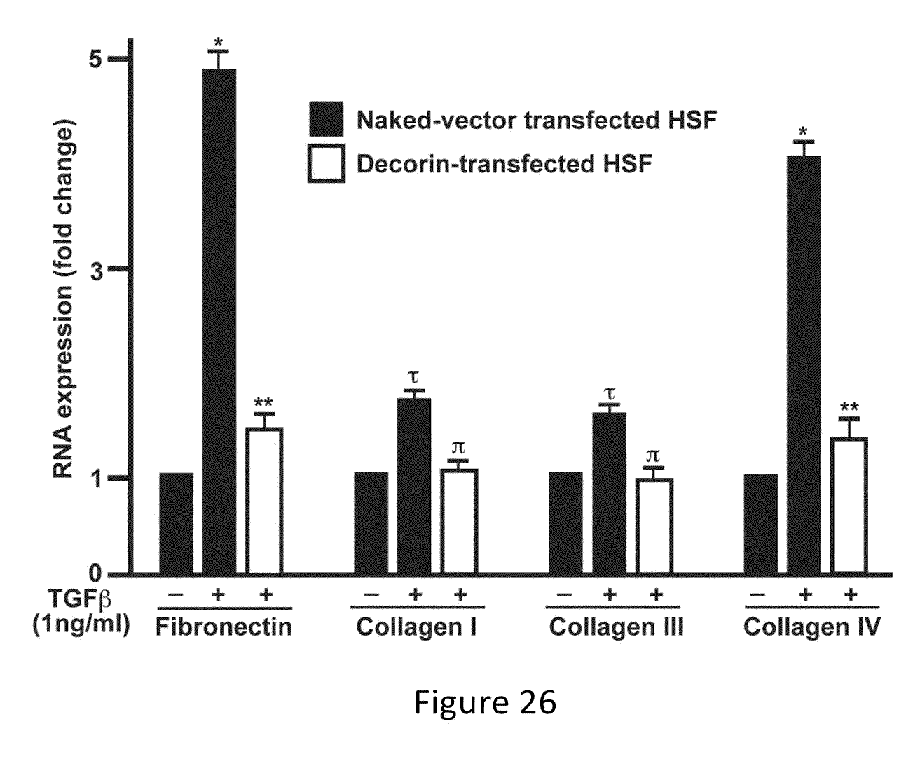

Quantitative PCR investigated mRNA expression of the extracellular matrix (ECM) components namely fibronectin, collagen type I, III and IV in decorin- or naked-vector transfected HSF clones grown in +/− of TGFβ1 (1 ng/ml) under serum-free conditions or 10% serum-containing medium. FIG. 26 presents quantitative mRNA measurements performed with real-time PCR. The TGFβ1 stimulation caused statistically significant increase in the expression of tested pro-fibrogenic genes fibronectin (4.8-fold; p<0.01), collagen type I (1.6-fold; p<0.05), collagen type III (1.4-fold; p<0.05), and collagen type IV (4.1-fold; p<0.01) in naked vector-transfected HSF cultures. The decorin transfection to HSF significantly inhibited TGFβ1-driven increase of ECM as decorin-transfected HSF cultures showed significant decrease in fibronectin (3.1-fold; p<0.001), collagen type I (1-fold; p<0.01), collagen type III (0.9-fold; p<0.01), and collagen type IV (3.2-fold; p<0.001) RNA levels compared to the naked-vector transfected HSF cultured under similar conditions (FIG. 26). No significant differences in the fibronectin, collagen type I, III or IV RNA levels were detected between the un-transfected normal and naked-vector transfected HSF cultures.

Effect of Decorin Transfection on TGFβ1-Driven Myofibroblast Modulation

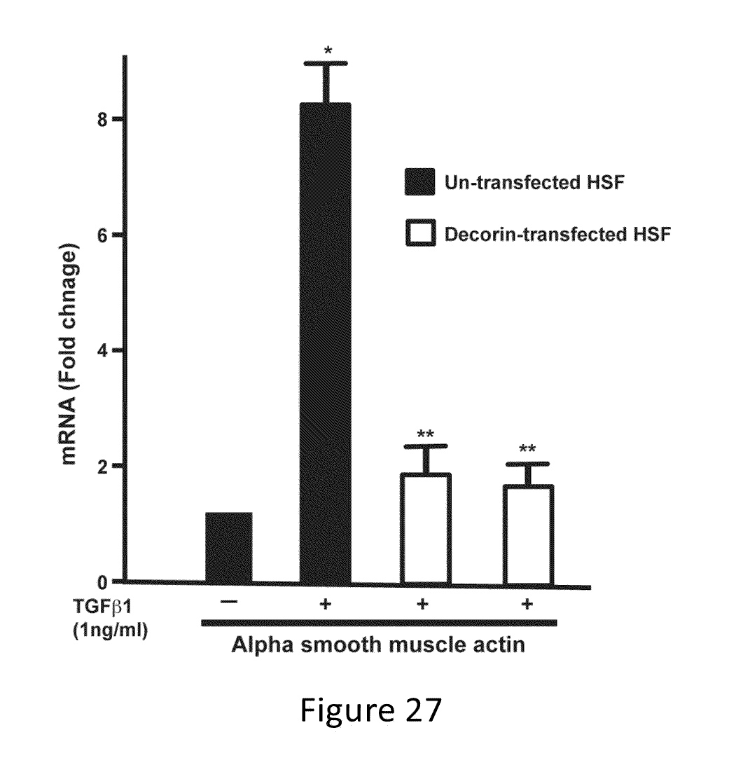

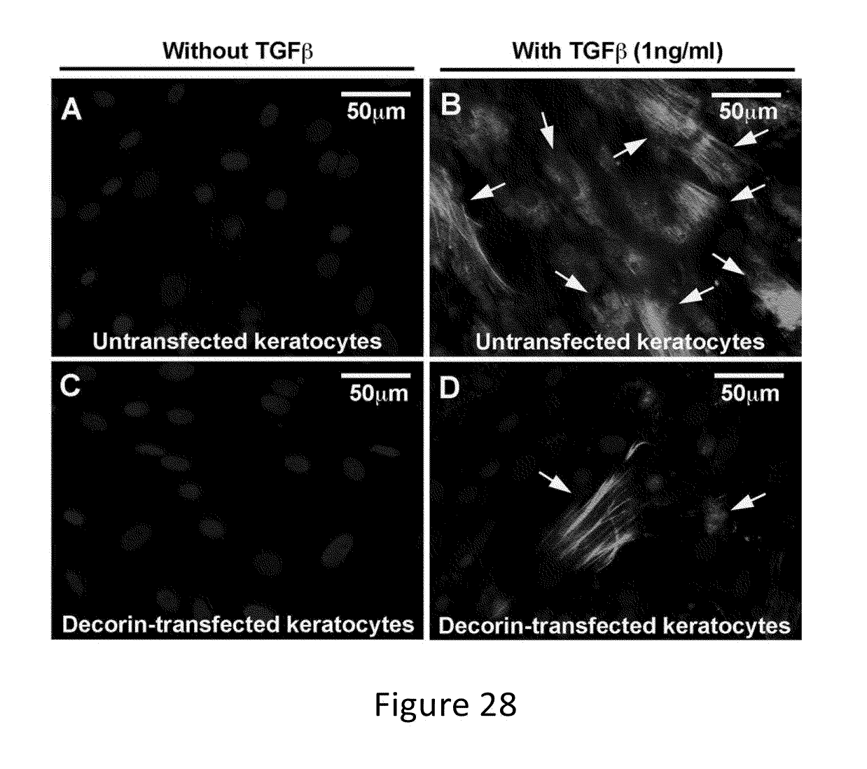

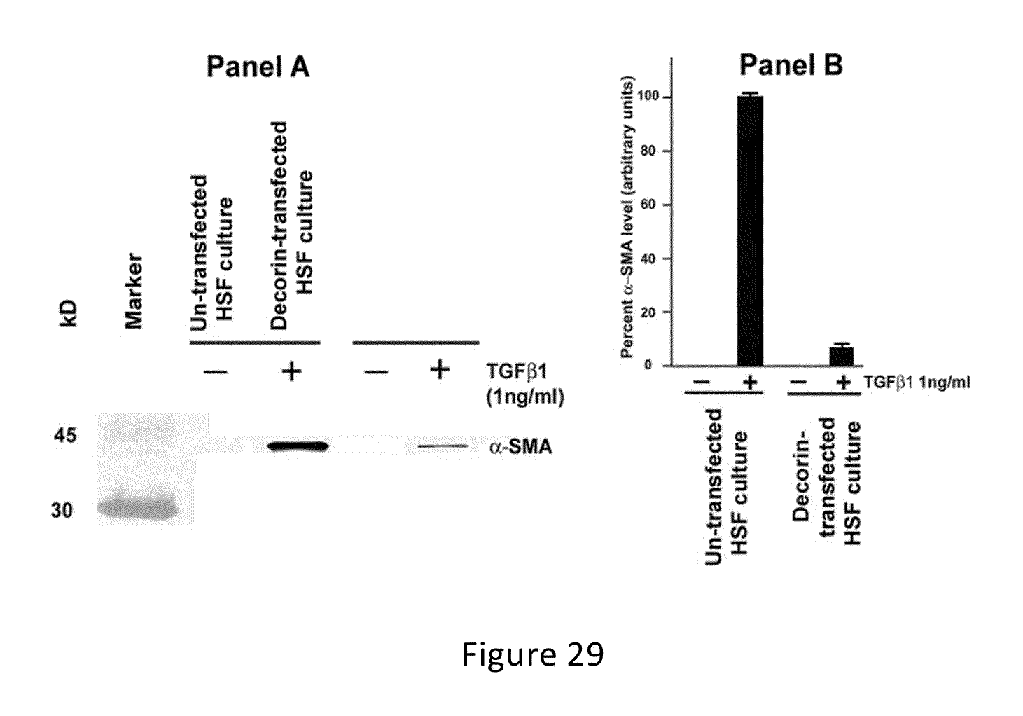

To test the hypothesis that decorin gene transfer inhibits myofibroblast formation in the cornea, the effect of decorin on TGFβ1-stimulated transformation of in corneal fibroblast to myofibroblasts was examined. The levels of a-smooth muscle actin (SMA), a myofibroblast marker, mRNA and protein in decorin-transfected and untransfected HSF clones grown in presence or absence of TGFβ1 (1 ng/ml) under serum-free conditions were quantified with immunostaining and immunoblotting. FIG. 27 shows quantitative measurement of SMA RNA in decorin-transfected and un-transfected HSF cultures. As evident from the data TGFβ1 stimulated un-transfected HSF culture showed 8.3 fold (p<0.01) increase in the levels of SMA gene expression compared to HSF not stimulated to TGFβ1, and decorin-transfected HSF cultures demonstrated significant decrease in SMA (more than 6-fold; p value <0.001) under similar conditions (FIG. 27). FIGS. 28 and 29 show analysis of SMA protein performed with immunocytochemistry and western blot, respectively. As evident from the immunocytochemistry data shown in FIG. 27, decorin-transfected HSF clone (panel D) showed a statistically significant lowering in SMA-stained cells (˜79%, p value<0.01) compared to the untransfected normal HSF grown in the presence of TGFβ1 (panel B) under serum-free conditions. Conversely, neither untransfected normal (panel A) nor decorintransfected (panel C) cultures grown in absence of TGFβ1 showed any SMA-stained cells (FIG. 28). Similar results were observed with western blot analysis and are shown in FIG. 29. The whole cell lysates of decorin-transfected HSF cultures showed a statistically significant decrease in SMA levels (−83%, p value <0.01) compared to un-transfected HSF cultures. These findings support our hypothesis that decorin gene transfer could inhibit myofibroblast and haze development in the cornea in vivo.

### Example 5

### Material and Methods

AAV Vector Production

Plasmids pCMV-Cap6, pMT-Rep2 and AAV genomic plasmid, pARAP4 were obtained from Dr Dusty Miller, Fred Hutchison Cancer Research Center, Seattle Wash. Plasmids pAAV2/8, pAAV2/9 were provided by Dr James M. Wilson, Gene Therapy Program, Division of Medical Genetics, Department of Medicine, University of Pennsylvania, Philadelphia Pa. AAV vectors were generated using adenovirus free system following previously published protocol (Ghosh et al., 2006). Briefly, human embryonic kidney (HEK) 293 cells were co-transfected with AAV2-based genomic vector pARAP4, AAV Rep/Cap plasmids and adenovirus helper plasmid (pHelper; Stratagene La Jolla, Calif., cat.#240071). The pARAP4 expresses heat stable placental alkaline phosphatase (AP) under the regulation of Rous sarcoma virus (RSV) promoter/enhancer and simian virus 40 (SV40) polyadenylation sequence. AAV6 was generated using four plasmids viz pHelper, pCMV-Cap6, pMT-Rep2 and pARAP4. These plasmids were used at a ratio of 3:3:1:1 respectively. For AAV 8 and AAV9 production, pAAV2/8 and pAAV2/9 encoding for AAV2rep and AAV8 or AAV9cap were used along with pHelper and pARAP4 at a ratio of 3:3:1 respectively. The virus containing cell lysate was harvested at 62 h post-transfection. Recombinant viral stocks were purified by two sequential rounds of CsCl gradient ultracentrifugation. Collected viral fractions were pooled and dialyzed through two rounds of HEPES-buffered saline. Viral titer was determined by dot blot analysis using DIG labeled probes (Roche Applied Science, Indianapolis, Ind.).

AAV Transduction of Mouse and Human Corneas

All animals and human corneas were treated in accordance with the tenets of the ARVO Statement for the Use of Animals in Ophthalmic and Vision Research and the declaration of Helsinki. Six to eight weeks old female C57 mice were used for the study. The study was approved by the Institutional Animal Care and Use Committee (Harry S. Truman Memorial Veterans' Hospital, Columbia, Mo.). Mice were anaesthetized with intramuscular injection of ketamine (130 mg/kg) and xylazine (8.8 mg/kg). Topical solution of 1% proparacaine hydrochloride (Alcon, Ft. Worth, Tex.) was instilled to each eye for local anesthesia. Alcohol soaked gauze was applied on each cornea for 20 s and epithelium was removed by gentle scraping with a #64 Beaver blade (BectoneDickinson, Franklin Lakes, N.J.) under an operating microscope. Two microliters of viral vector (viral titer 109 genomic copies/ml) was directly applied to the corneal stroma for 2 min after drying the cornea with merocel sponge. Animals were divided into 3 groups. The eyes of Group 1 received AAV6 vector, Group 2 received AAV8 vector and Group 3 received AAV9 vector. Animals of each group were sacrificed at 4, 14 or 30 days after AAV application.

For ex vivo human cornea application, the stroma of donor human corneas were incubated with AAV serotypes (viral titer 10⁹ vg/ml) in a humidified CO₂ incubator at 37° C. in Dulbecco's modified Eagle's medium containing 10% fetal bovine serum for 12 h. Thereafter, the tissues were washed with HBSS twice and were incubated for another 5 days in growth medium.

Tissue Embedding

Mouse and human corneal tissues were embedded in liquid OCT compound (Sakura FineTek, Torrance, Calif.) within a 15 mm×15 mm×5 mm mold (Fisher, Pittsburgh, Pa.) and snap frozen as reported previously (Mohan et al., 2003). The frozen

tissue blocks were maintained at −80° C. Seven micron thick tissue sections were cut with a cryostat (HM 525M, Microm GmbH, Walldorf, Germany) and maintained frozen at −80° C. until staining.

Alkaline Phosphatase Detection, Localization and Quantification in Corneal Tissues

Corneal sections were washed with HEPES buffer. Cytochemical staining was performed by incubating the corneal sections with a mixture of BCIP (5-Bromo-4-Chloro-30-Indolylphosphate p-toluidine) and NBT (Nitro-Blue Tetrazolium) at 37° C. for 10 min. The AP-stained corneal stroma appeared dark blue. The nuclei were stained using nuclear red fast solution (Sigma Aldrich Inc., St. Louis, Mo.). To determine the levels of transgene delivery in the cornea mean pixel area of AP staining in six randomly selected, non-overlapping, full-thickness corneal sections (4×104 μm²) was quantified.

Alkaline Phosphatase Enzyme Activity in Corneal Lysate

The corneal lysates were prepared using RIPA buffer (Tris 50 mM, NaCl 150 mM, NP40 1%, Na-deoxycholate 0.5% containing 1× protease inhibitor). The protein content for each sample was determined using the Bradford assay. AP activity in corneal lysates was determined by a spectrophotometric assay using StemTAG alkaline phosphatase activity assay kit (Cell Biolabs, Inc., San Diego, Calif.) following the manufacturer's protocol. Controls were a blank sample (no corneal lysate) and corneal lysate from control eyes. The optical density for AP activity was read at 405 nm. The p-nitrophenol was used for plotting the standard curve. The AP activity was expressed as μM of p-nitrophenol generated/mg protein. AP activity assay was performed on three corneas for each time point and each sample was analyzed in duplicate.

TUNEL Assay

For TUNEL assay, tissue sections were fixed in acetone at −20° C. for 10 min, dried at room temperature for 5 min, and then placed in PBS balanced salt solution. Fluorescent ApopTag apoptosis detection assay (Chemicon international, Temecula Calif.) that predominantly detects apoptosis and to a lesser extent necrosis, was performed following the manufacturer's instructions. Appropriate positive (corneal scrape) and negative (unwounded) controls were included in each assay.

Cd11b and F4/80 Immunostaining

Immunofluorescent staining for CD11b (BD Pharmingen, San Jose, Calif.) and F4/80 (Serotec, Raleigh, N.C.) was performed using rat anti-mouse antibodies. Tissue sections (7 μm) were incubated at room temperature with the primary antibody at 1:50 dilution in 1×HEPES containing 5% BSA for 90 min and with secondary antibody goat anti-rat IgG (AlexaFlour 594, Molecular Probes, Eugene, Oreg.) at a dilution of 1:500 for 60 min. Vectashield mounting medium containing DAPI (Vector Laboratories, Inc. Burlingame, Calif.) was used to visualize nuclei in the tissue sections. The sections were viewed and photographed under a Leica fluorescent microscope (Leica, Wetzlar, Germany) equipped with a digital camera (SpotCam RT KE, Diagnostic Instruments Inc., Sterling Heights, Mich., USA).

Statistical Analysis

The results were expressed as mean±standard error of the mean (SEM). Statistical analysis between various AAV serotypes was performed using two-way analysis of variance (ANOVA) followed by Bonferroni test. For comparing different time points within a serotype, one way ANOVA followed by Tukey's multiple comparison test was used. A p value <0.05 was considered as statistically significant.

### Results

Level, Localization and Duration of AAV-Mediated Gene Transfer in Mouse Cornea

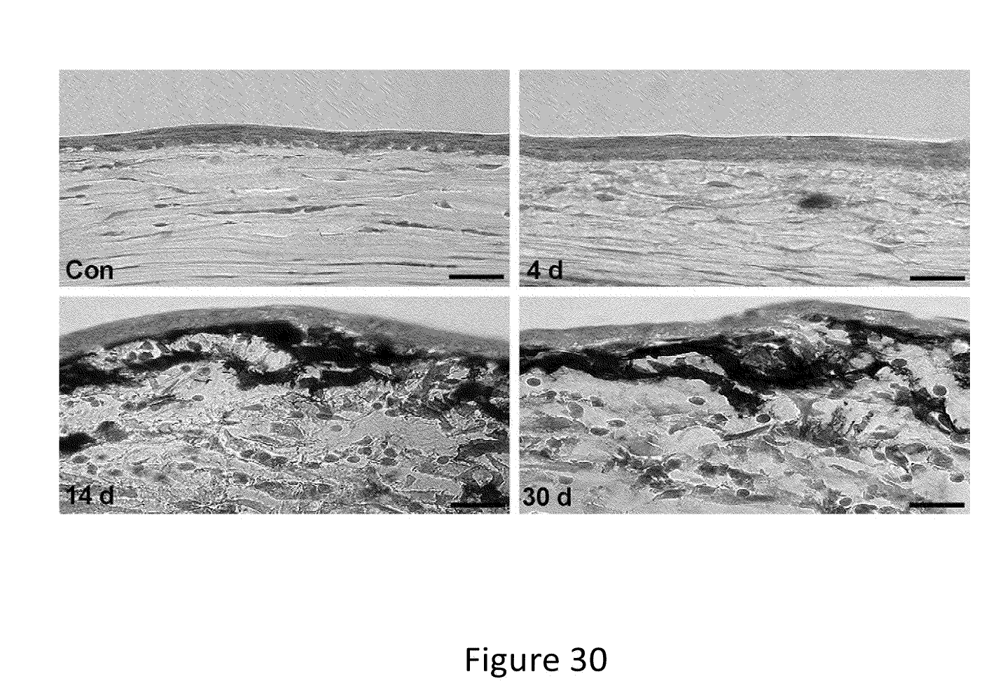

Alkaline phosphatase (AP) staining representing level and localization of gene transfer in corneal sections of mouse eyes treated with AAV9, AAV8 and AAV6 is shown in FIGS. 30 to 32. As evident from these figures a statistically significant transgene delivery was noted in the anterior stroma below the epithelium of mouse corneas treated with AAV than the control corneas (p<0.01 or p<0.05). No AP staining was detected either in the epithelium or endothelium suggesting that vector-delivery technique plays a critical role in tissue-selective gene delivery as reported earlier (Mohan et al., 2005a).

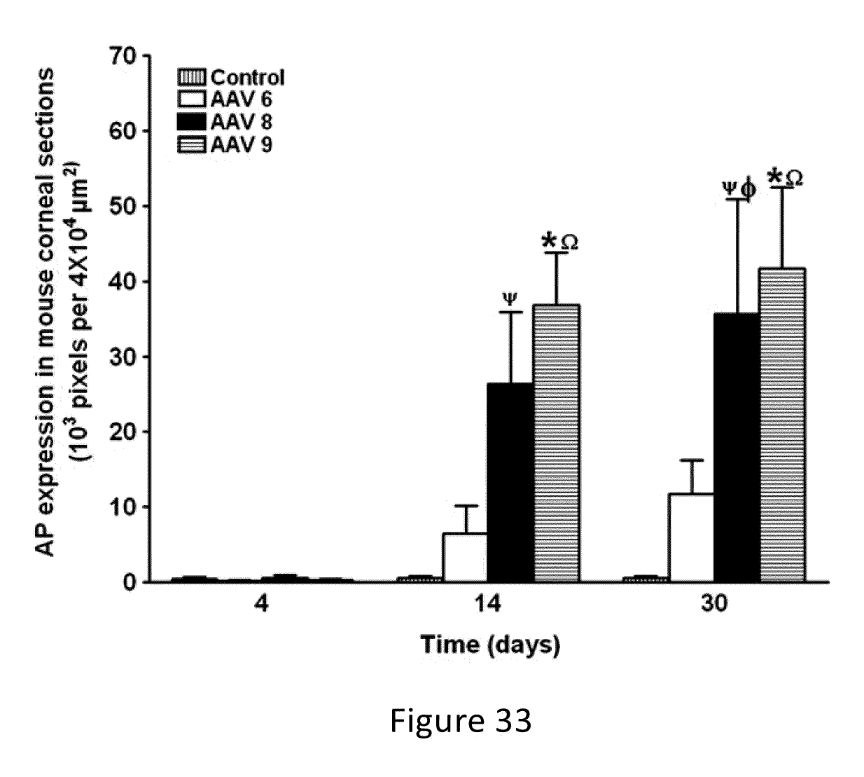

FIG. 30 shows transgene delivery in the mouse cornea with AAV9 vector examined on 3 selected time points (4, 14 and 30 days). The testing of AAV9-mediated gene transfer earlier than day 4 time point was not done because our earlier experiments showed that the onset of gene expression with AAV2 and AAV5 starts from day 3 (Mohan et al., 2005, 2005a). The AAV9 showed several fold higher transgene expressions in mouse stroma on day 14 and 30 compared to day 4. The amount of transgene delivery was significantly higher at day 14 and 30 compared to day 4 (p<0.01; shown by * in FIG. 33). This suggests that AAV9 mediated transgene expression reached its peak on or before day 14 and continued to express up to the longest tested time point of 30 days.

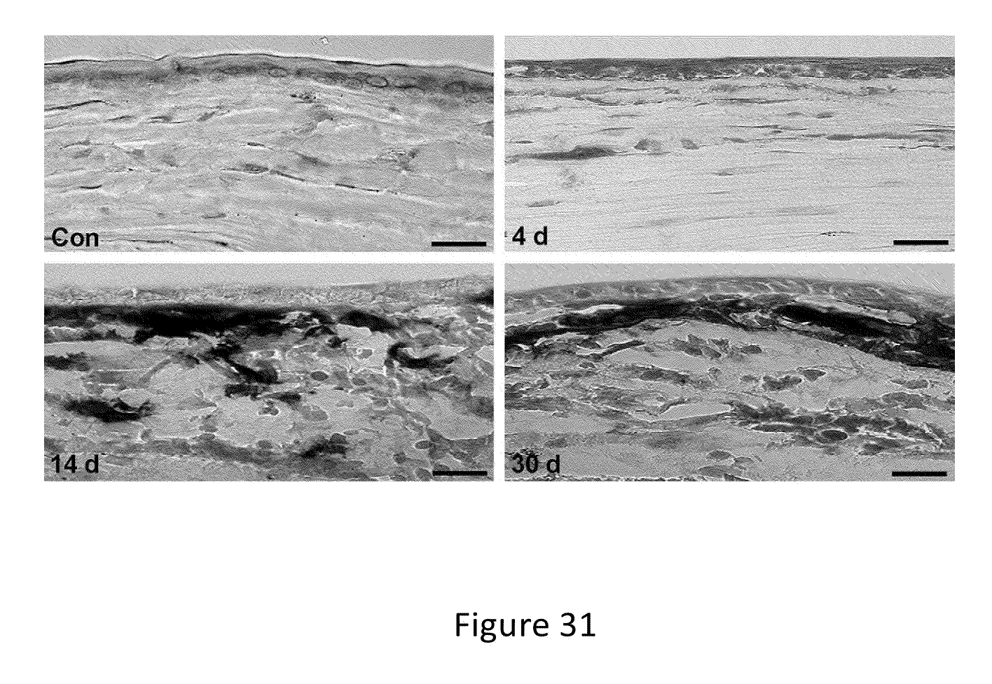

FIG. 31 demonstrates the level and localization of AAV8 mediated transgene expression in mouse corneal sections collected on day 4, 14 and 30. AAV8 serotype also showed significantly high amount of transgene delivery into keratocytes of the mouse cornea on day 14 and 30 compared to day 4 (p<0.05; shown by ψ in FIG. 33). The transduction characteristics exhibited by the serotype 8 was similar to the serotype 9 showing peak transgene expression on tested day 14 that continued up to day 30.

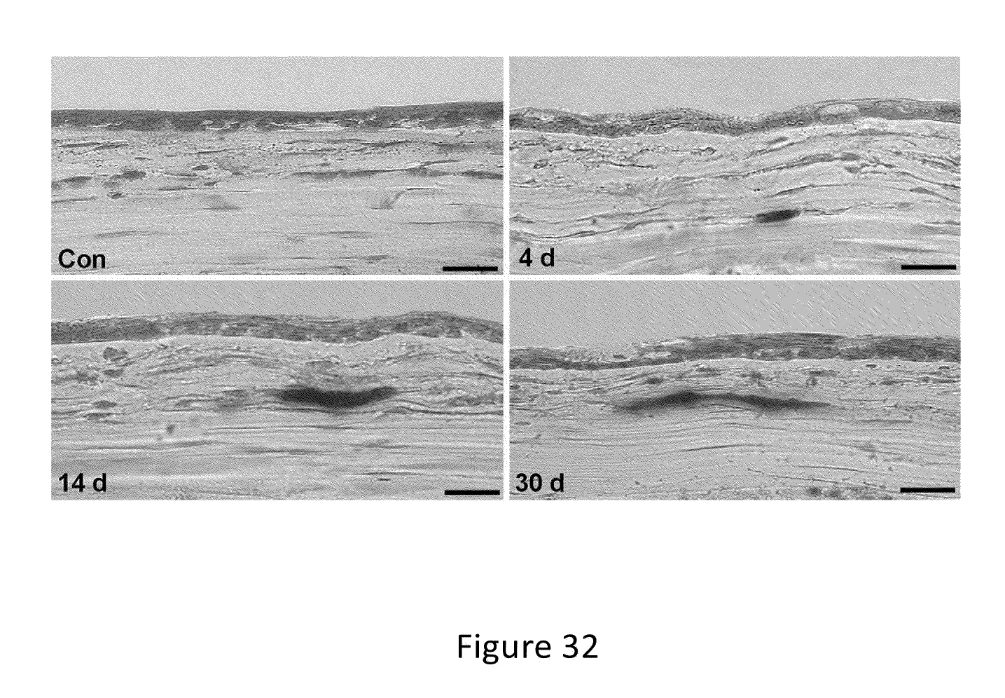

FIG. 32 depicts the gene transfer data collected with AAV6 from corneal sections of mouse eyes treated with AAV6 on days 4, 14 and 30. Contrary to AAV serotypes 8 and 9, serotype 6 showed low to moderate level of transgene delivery in the mouse stroma and the transgene expression was localized more towards the posterior stroma. The transgene expression on days 14 and 30 was more than the day 4, however, it was statistically insignificant (p>0.05).

Cytochemical Quantification of AAV Transduction Efficient in Mouse Cornea

The amount of transgene delivery quantified using Image J program showed relative transduction efficiency of the tested AAV serotypes and has been shown in FIG. 33. The three AAV serotypes showed significant transgene delivery as compared to control corneas. The highest transgene delivery was noted with AAV9 and the lowest transduction efficiency was noted with AAV6. The relative order of transduction efficiency was AAV9>AAV8>AAV6. The AAV9-treated corneas showed significantly higher transgene expression compared to AAV6 (3.5-5.5 fold, p<0.01; shown by Ω in FIG. 33) and AAV8 (1.1-1.4 fold, p>0.05 not significant) at days 14 and 30. Relative comparison between AAV serotype 8 and 6 was also significantly greater (3.1-4 fold, p<0.05; shown by φ in FIG. 33) at days 14 and 30.

Functional Assay Quantifying AAV Transduction Efficiency in Mouse Cornea

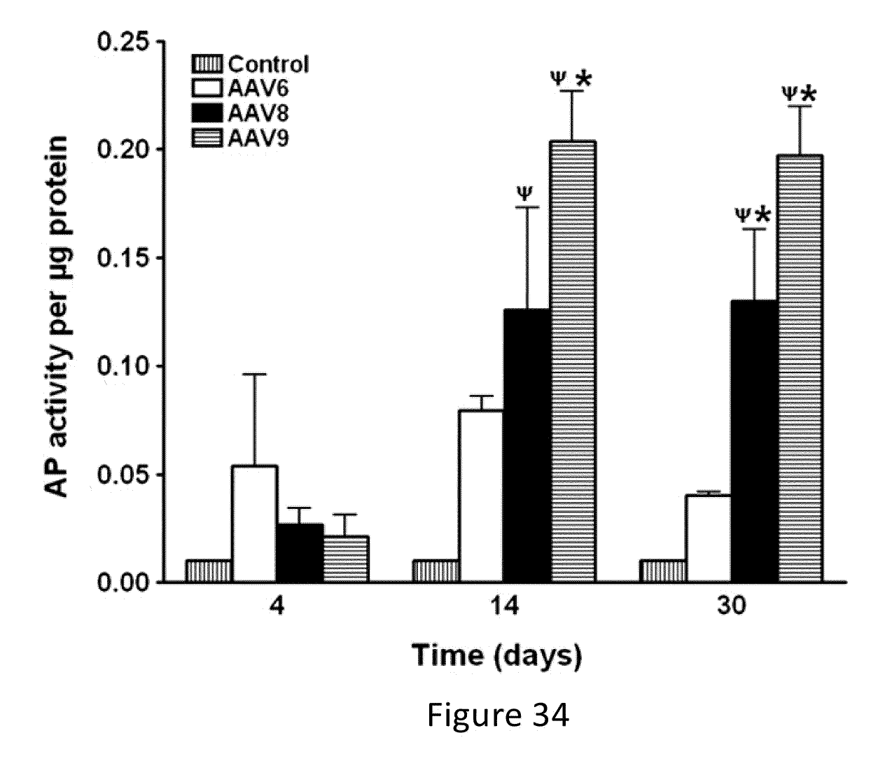

The biological function of a delivered transgene was determined using AP enzyme functional assay. The transduction efficiencies of the tested serotypes were also compared by quantifying the AP enzyme activity in the corneal homogenates. FIG. 34 demonstrates the AP enzyme activity in the corneal homogenates prepared from mouse eyes treated with selected AAV serotypes at 3 tested time points. All tested serotypes demonstrated markedly higher AP enzyme activity at the 3 selected time points (4, 14 and 30 days) compared to controls. The level of enzyme activity at day 14 and day 30 was significantly higher as compared to day 4 in AAV8 and AAV9 treated corneal homogenates (p<0.05; shown by ψ in FIG. 34).

Amongst the three serotypes, AAV9 treated corneal homogenates showed highest levels of AP enzyme activity and it was 2.5 fold (p<0.05; shown by * in FIG. 34) higher as compared to AAV6 and 1.5 fold (p>0.05; not significant) higher than AAV8. Comparative analysis between AAV8 and AAV6 revealed that AP enzyme activity was 1.7 fold higher (p<0.05; shown by * in FIG. 34) for AAV8 treated corneas than AAV6.

Gene Transfer in Human Cornea with AAV Serotypes

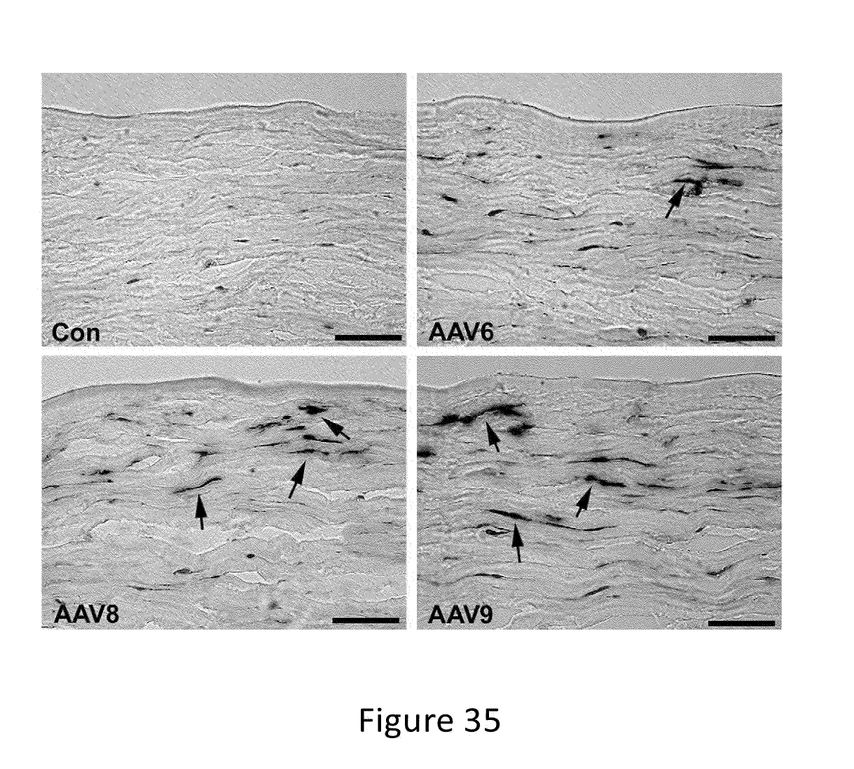

FIG. 35 demonstrates gene transfer efficiency of the three tested AAV serotypes in human cornea ex vivo. Serotype 9 showed the highest and serotype 6 showed the lowest amount of gene transfer in the human cornea. AAV 8 serotype demonstrated significantly higher gene delivery compared to AAV 6 but moderately less than AAV9. This experiment was performed because tested AAV serotypes showed different transduction efficiencies for mouse cornea in vivo and human cornea in vitro (Sharma et al., 2010). Thus, we wanted to know whether selected AAV serotypes transduce donor human cornea according to transduction pattern that was observed in vitro or in vivo. This data suggests that AAV serotypes follow transduction pattern noted for the mouse cornea in vivo suggesting that in vivo testing is critical for establishing vector efficacy for gene therapy. Limited availability of donor human corneas restricted us from performing statistical analysis.

Effect of Titer on AAV-Mediated Gene Transfer in Mouse Cornea

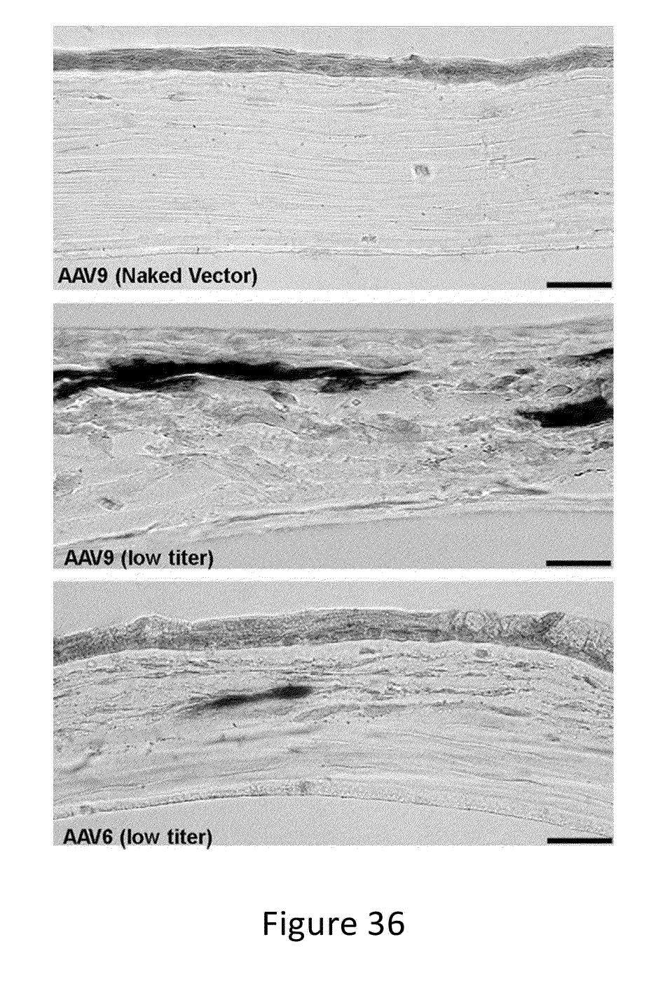

To test the effect of viral titer on the level of transgene expression, we applied 1,000 times diluted AAV9 to the mouse cornea. As shown in FIG. 36, mouse cornea treated with the low titer AAV9 serotype (10⁶ genomic copies/μl) resulted in a notable decrease in the level of transgene expression.

Effect of AAV on Cell Death and Immune Reaction

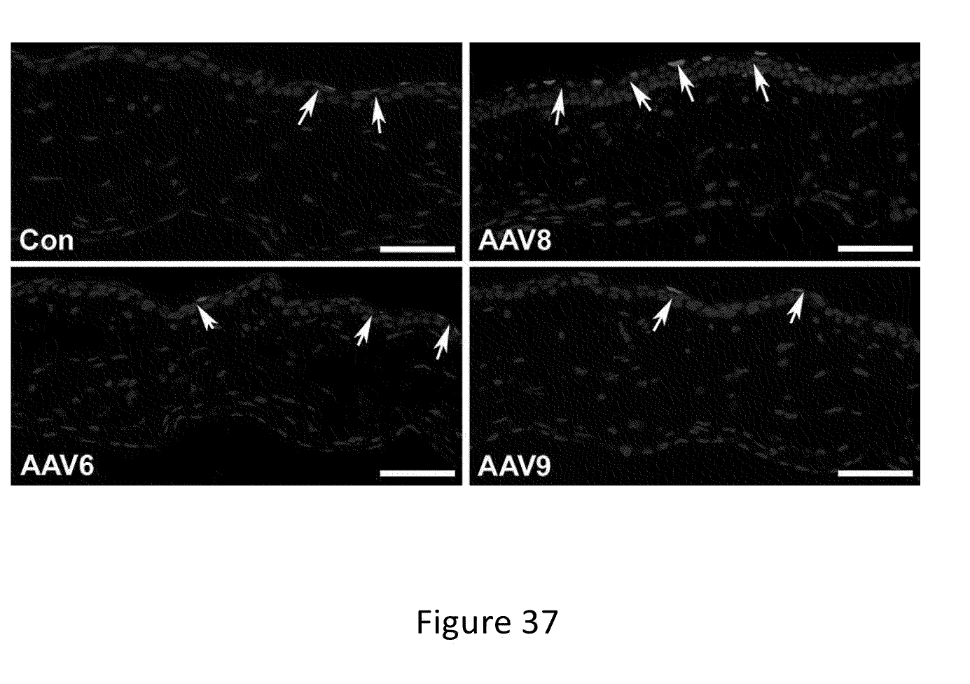

FIG. 37 shows results of TUNEL staining in control and AAV treated mouse corneas 4 days after epithelial scraping and viral vector application. In control corneas, TUNEL positive cells were mostly noted in corneal epithelium with no or very few TUNEL positive cells in stroma. None of the three AAV serotypes caused any significant change in TUNEL positive cells in mouse corneal sections. To further rule out the possibility of late onset cellular toxicity, we also performed the TUNEL staining at 14 days after AAV applications. No TUNEL positive cells were detected in corneal stroma at 14 days (data not shown) suggesting that tested AAV serotypes do not induce cell death, and are safe for corneal gene therapy.

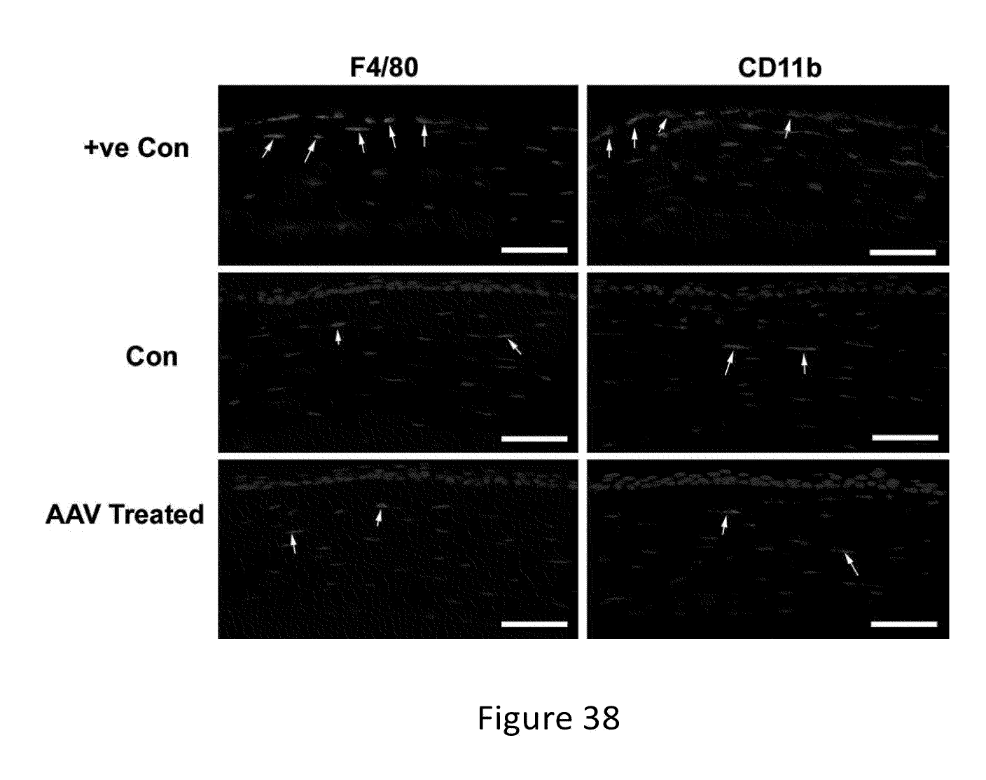

To test the possibility of inflammatory response due to AAV application, we stained the mouse corneal sections for CD11b, a granulocyte marker, and F4/80, a macrophage marker. As evident from FIG. 38, few CD11b or F4/80 positive cells were detected in control corneas at 4 days after epithelial scraping suggesting a normal corneal response to tissue injury. Topical application of AAV6, AAV8 or AAV9 did not cause any significant change in the number of CD11b or F4/80 stained cells. FIG. 37 shows a representative CD11b and F4/80 staining in mouse corneas treated with AAV9 at day 4 after vector application. Similar level of positive staining was noted with AAV6 or AAV8.

What is claimed is:

1. A method of delivering a gene to a desired area of stroma of a cornea, the method comprising the steps of:
(a) preparing the cornea by:
(i) removing corneal tissue to expose at least a portion of the corneal stroma, and
(ii) dehydrating the exposed portion of the stroma;
(b) applying a viral vector that comprises the gene to the dehydrated portion of the stroma.

2. The method of claim 1 further comprising the step of removing excess viral vector after application in step (b).

3. The method of claim 1 wherein the viral vector is an AAV vector in solution with a titer of from about 1×10⁸ vg/ml to about 6.5×10¹² vg/ml.

4. The method of claim 1 wherein the viral vector is in solution and is applied in a volume of solution of from about 1 μl to about 100 μl.

5. The method of claim 1 wherein the viral vector is an AAV vector in solution with a titer of from about 1×10⁸ vg/ml to about 6.5×10¹² vg/ml and the AAV vector is applied in a volume of solution of from about 1 μl to about 100 μl.

6. The method of claim 1 wherein removing the corneal tissue in (a)(i) comprises mechanical scraping.

7. The method of claim 1 wherein dehydrating the exposed portion of the stroma in (a)(ii) comprises contacting the surface of the cornea with a flow of air at a temperature in the range of from about 40° C. to about 45° C. for a total duration of from about 10 seconds to about 60 seconds, wherein said flow is at a rate in the range of about 6 meters per second to about 10 meters per second.

8. The method of claim 1 wherein following preparation of the cornea in (a), a physical barrier is placed on the cornea that encompasses the desired surface area of stroma of the cornea to which the gene is to be delivered, and wherein the viral vector applied in (b) is in a solution that is applied to the area encompassed by the physical barrier.

9. A method of treating corneal scarring comprising applying a viral vector that comprises a TGFβ-antagonizing gene to the stroma of a cornea.

10. The method of claim 9 wherein the viral vector is an AAV vector.

11. The method of claim 9 wherein the TGFβ-antagonizing gene is decorin.

12. The method of claim 9 wherein the TGFβ-antagonizing gene is delivered to a desired area of stroma of the cornea, the method comprising the steps of:
(a) preparing the cornea by:
(i) removing corneal tissue to expose at least a portion of the corneal stroma, and
(ii) dehydrating the exposed portion of the stroma;
(b) applying an AAV viral vector comprising the TGFβ-antagonizing gene to the dehydrated portion of the stroma.

13. The method of claim 12 wherein the AAV vector is in solution with a titer of from about 1×10⁸ vg/ml to about 6.5×10¹² vg/ml.

14. The method of claim 12 wherein the viral vector is in solution and is applied in a volume of solution of from about 1 μl to about 100 μl.

15. The method of claim 12 further comprising the step of removing excess AAV vector after application in step (b).

16. The method of claim 12 wherein the TGFβ-antagonizing gene is decorin.

17. The method of claim 12 wherein removing the corneal tissue in (a)(i) comprises mechanical scraping.

18. The method of claim 12 wherein dehydrating the exposed portion of the stroma in (a)(ii) comprises contacting the surface of cornea with a flow of air at a temperature in the range of from about 40° C. to about 45° C. for a total duration of from about 10 seconds to about 60 seconds, wherein said flow is at a rate in the range of about 6 meters per second to about 10 meters per second.

19. The method of claim 12 wherein following preparation of the cornea in (a), a physical barrier is placed on the cornea that encompasses the desired surface area of stroma of the cornea to which the gene is to be delivered, and wherein the viral vector applied in (b) is in a solution that is applied to the area encompassed by the physical barrier.

20. A method of treating corneal scarring comprising applying an AAV5 vector that comprises a decorin gene to the stroma of a cornea wherein the gene is delivered to a desired area of stroma of a cornea, the method further comprising the steps of:
(a) preparing the cornea by:
(i) removing corneal tissue by mechanical scraping to expose at least a portion of the corneal stroma, and
(ii) dehydrating the exposed portion of the stroma;
(b) applying the AAV5 vector that comprises the decorin gene to the dehydrated portion of the stroma,
wherein the AAV5 vector is in solution with a titer of from about 1×10⁸ vg/ml to about 6.5×10¹² vg/ml and is applied in a volume of solution of from about 1 μl to about 100 μl,
wherein dehydrating the exposed portion of the stroma in (a)(ii) comprises contacting the surface of the cornea with a flow of air at a temperature in the range of from about 40° C. to about 45° C. for a total duration of about 10 seconds to about 60 seconds,
wherein said flow is at a rate in the range of about 6 meters per second to about 10 meters per second, and
wherein following preparation of the cornea in (a), a physical barrier is placed on the cornea that encompasses the desired surface area of stroma of the cornea to which the gene is to be delivered, and wherein the viral vector applied in (b) is applied to the area encompassed by the physical barrier.


SEQUENCE LISTING (6 sequences)

SEQ ID NO: 1 - protein, 11 aa, Artificial Sequence
  Consensus
LXXLXLXXNXL

SEQ ID NO: 2 - DNA, 33 bp, Artificial Sequence
  Primer Sequence
gatcgcggccgcaatcatgacggcaactctcat

SEQ ID NO: 3 - DNA, 34 bp, Artificial Sequence
  Primer sequence
gtcagcggccgcgagttacttgtagtttccgagc

SEQ ID NO: 4 - DNA, 1553 bp, Oryctolagus cuniculus
tacatgaaataaatcatgacggcaactctcatcctccttctgcttgcacaagtttcctgggccgggccgtttcaacagagaggcttatttgactttatgctagaagatgaagcttctgggataggcccagacgagagagctcctgaacttcccgacttggacatgctgggaccagtgtgtcctttccgctgtcagtgtcaccttcgagttgtccagtgttctgatttgggtctggacaaagtgcccaaggaccttccccctgacacgacgctactggacctgcaaaacaacaaaataactgaaatcaaagatggagattttaagaacctgaagaaccttcacgcattgatcctcgtcaacaacaaaattagcaaaatcagccctggagcatttacacctttggtgaaattggagcgactttatttgtctaagaaccatctgaaagaattgccagaaaaaatgcccaaatctcttcaagagctgcgtgcccatgagaatgagatcacaaaagtacgaaaatctgtgttcagtggaatgaaccagatgattgtcatagaactgggcaccaaccctctgaagagctcaggaattgaaaatggagccttccagggaatgaagaagctctcctacattcgtattgctgataccaacataaccaccatccctcaaggtcttcctccttctcttacggaactacatcttgatggcaataaaatcaccaaaatcgatgcatctagcctgaaaggactgaataatttggctaagttgggtctgagtttcaatgacatctctgctgtggacaatggttctctggccaatgcccctcatctgcgagagcttcacttggacaacaacaaacttatccgagtacctggtggtctggcagatcataaatacattcaggttgtctaccttcataacaataatatctctgtagttggagcaaatgacttctgccctcctgggtacaacaccaaaaaggcttcctactcaggtgtgagtcttttcagcaaccctgtgcaatactgggagatccagccatccaccttcagatgcgtctacatgcgctctgccattcagctcggaaactacaagtaactcccaagaacaccctcatgtttataacctggcaaaatcctgttaatgtcattgctaaaactaaaaataaaataaagaataaaagctagatactgggaacctaactgcaatgtggatatttttcccacatgacttatgcataaagctaaatatgcagtttaaataattgcctacaataaaaaaattgcctgccattttgagaatcatttttgaagttttctgttgatgttaactgagctaccacaattaatcttaatttcactaattgtaaaattttgagtaaatatacatatatcaataatttagtaaagtgtttcttttttaattttcagaatgaatcaaataaagagtgttggtcttttgtaattcataggtcagttagctttgttgacataaagtgaaatacgatatactaagtgtgtattaacccaccactgttactgctaaagccacac

SEQ ID NO: 5 - DNA, 2305 bp, Homo sapiens
gaatctacaataagacaaatttcaaatcaagttgctccactatactgcataagcagtttagaatcttaagcagatgcaaaaagaataaagcaaatgggaggaaaaaaaaggccgataaagtttctggctacaatacaagagacatatcattaccatatgatctaatgtgggtgtcagccggattgtgttcattgagggaaaccttattttttaactgtgctatggagtagaagcaggaggttttcaacctagtcacagagcagcacctaccccctcctcctttccacacctgcaaactcttttacttgggctgaatatttagtgtaattacatctcagctttgagggctcctgtggcaaattcccggattaaaaggttccctggttgtgaaaatacatgagataaatcatgaaggccactatcatcctccttctgcttgcacaagtttcctgggctggaccgtttcaacagagaggcttatttgactttatgctagaagatgaggcttctgggataggcccagaagttcctgatgaccgcgacttcgagccctccctaggcccagtgtgccccttccgctgtcaatgccatcttcgagtggtccagtgttctgatttgggtctggacaaagtgccaaaggatcttccccctgacacaactctgctagacctgcaaaacaacaaaataaccgaaatcaaagatggagactttaagaacctgaagaaccttcacgcattgattcttgtcaacaataaaattagcaaagttagtcctggagcatttacacctttggtgaagttggaacgactttatctgtccaagaatcagctgaaggaattgccagaaaaaatgcccaaaactcttcaggagctgcgtgcccatgagaatgagatcaccaaagtgcgaaaagttactttcaatggactgaaccagatgattgtcatagaactgggcaccaatccgctgaagagctcaggaattgaaaatggggctttccagggaatgaagaagctctcctacatccgcattgctgataccaatatcaccagcattcctcaaggtcttcctccttcccttacggaattacatcttgatggcaacaaaatcagcagagttgatgcagctagcctgaaaggactgaataatttggctaagttgggattgagtttcaacagcatctctgctgttgacaatggctctctggccaacacgcctcatctgagggagcttcacttggacaacaacaagcttaccagagtacctggtgggctggcagagcataagtacatccaggttgtctaccttcataacaacaatatctctgtagttggatcaagtgacttctgcccacctggacacaacaccaaaaaggcttcttattcgggtgtgagtcttttcagcaacccggtccagtactgggagatacagccatccaccttcagatgtgtctacgtgcgctctgccattcaactcggaaactataagtaattctcaagaaagccctcatttttataacctggcaaaatcttgttaatgtcattgctaaaaaataaataaaagctagatactggaaacctaactgcaatgtggatgttttacccacatgacttattatgcataaagccaaatttccagtttaagtaattgcctacaataaaaagaaattttgcctgccattttcagaatcatcttttgaagctttctgttgatgttaactgagctactagagatattcttatttcactaaatgtaaaatttggagtaaatatatatgtcaatatttagtaaagcttttcttttttaatttccaggaaaaaataaaaagagtatgagtcttctgtaattcattgagcagttagctcatttgagataaagtcaaatgccaaacactagctctgtattaatccccatcattactggtaaagcctcatttgaatgtgtgaattcaatacaggctatgtaaaatttttactaatgtcattattttgaaaaaataaatttaaaaatacattcaaaattactattgtatacaagcttaattgttaatattccctaaacacaattttatgaagggagaagacattggtttgttgacaataacagtacatcttttcaagttctcagctatttcttctacctctccctatcttacatttgagtatggtaacttatgtcatctatgttgaatgtaagcttataaagcacaaagcatacatttcctgactggtctagagaactgatgtttcaatttacccctctgctaaataaatattaaaactatcatgtgaaaaaaaaaaaaaaaaaa

SEQ ID NO: 6 - DNA, 1886 bp, Mus musculus
gggggagggagaagggggccgataaagtttctggctacaatacaagagacgtatcattaccatatgatctaatgtgggtgtcagctggatgcgctcacgcagtgaaaccttagttcttaactgtgctatggagtagaagcaggaggttttcaacccagtgacagtcacagggcagcaccaccccctcctccttctttccacacctgcaaacccttttgcctgggctgcatagttagtgttaattacatcgcagctttgagggctcctgtggcaaatacccggattaaaaggtcgtgaaaatacatgagataatcatgaaggcaactctcatcttcttccttctggcacaagtctcttgggctggaccatttgaacagagaggcttatttgacttcatgctagaagatgaggcttctggcataatcccttatgaccctgacaatcccctgatatctatgtgcccctaccgatgccagtgtcatcttcgagtggtgcagtgttctgatctgggtttggacaaagtgccctgggattttccacccgacacaaccttgctagacctgcaaaacaacaaaattacagagatcaaagaaggggccttcaagaacctgaaggacttgcataccttgatccttgtcaacaacaagatcagcaaaatcagtccagaggcattcaaacctctcgtgaagttggaaaggctttacctgtctaagaaccaactaaaggaactgcctgaaaaaatgcccagaactctccaggaacttcgtgtccatgagaatgagatcaccaagctgcggaaatccgacttcaatggactgaacaatgtgcttgtcatagaactgggcggcaacccactgaaaaactctgggattgaaaacggagccttccagggactgaagagtctctcatacattcgcatctcagacaccaacataactgcgatccctcaaggtctgcctacttctctcactgaagtgcatctagatggcaacaagatcaccaaggttgatgcacccagcctgaaaggactgattaatttgtctaaactgggattgagcttcaacagcatcaccgttatggagaatggcagtctggccaatgttcctcatctgagggaactccacttggacaacaacaaactcctcagggtgcctgctgggctggcacagcataagtatatccaggtcgtctaccttcacaacaacaacatctccgcagttgggcaaaatgacttctgccgagctggacacccctctcgaaaggcttcctactcggctgtgagtctttacggcaaccctgtccggtattgggaaatctttccaaacaccttcagatgtgtctatgtgcgttctgccattcaacttggaaactacaagtaaccctcagacggcctaattcttataatctggaaaaacaccccaatatgtcaatatcattgctaaaaagaaaaaatactaaaaaagaaagaaagaatgctagattctggaaattcaagcacactgtgcatgccttttccacatgacttattatgcaagctgaatatacagattgaataattgcctacaataaaaatattttacttgtagtattcagaatcacttttaaagttgtctgtagttgtgaactgagttatcaaagtctgatgtaatcataaatgtcaaccacttagtaaagcgttaaaaggaacagaatacaaagtctctgtaattcatagagctaaattaactcttttgacataaagtcaaatgccgccgaactctagcaatgtattaatctcctttattattggtgaagccttatttgagtatgtaagttcaatatgggctatgtaaaattttcactgtcatattgaaaaaaataaaattttaaaaatgcattcaaaagat